SEQUENCE LISTING (25 sequences)

SEQ ID NO: 1 - DNA, 1237 bp, Homo sapiens
gagaagcacttaattctacagcctccttcctagagccttcagtggcctctgccagtctggcagacacttgcagacctctcttctcagcaccaccaatctctgatgccctgcgatgcccacactcaatacttctgcctctccacccacattcttctgggccaatgcctccggaggcagtgtgctgagtgctgatgatgctccgatgcctgtcaaattcctagccctgaggctcatggttgccctggcctatgggcttgtgggggccattggcttgctgggaaatttggcggtgctgtgggtactgagtaactgtgcccggagagcccctggcccaccttcagacaccttcgtcttcaacctggctctggcggacctgggactggcactcactctccccttttgggcagccgagtcggcactggactttcactggcccttcggaggtgccctctgcaagatggttctgacggccactgtcctcaacgtctatgccagcatcttcctcatcacagcgctgagcgttgctcgctactgggtggtggccatggctgcggggccaggcacccacctctcactcttctgggcccgaatagccaccctggcagtgtgggcggcggctgccctggtgacggtgcccacagctgtcttcggggtggagggtgaggtgtgtggtgtgcgcctttgcctgctgcgtttccccagcaggtactggctgggggcctaccagctgcagagggtggtgctggctttcatggtgcccttgggcgtcatcaccaccagctacctgctgctgctggccttcctgcagcggcggcaacggcggcggcaggacagcagggtcgtggcccgctctgtccgcatcctggtggcttccttcttcctctgctggtttcccaaccatgtggtcactctctggggtgtcctggtgaagtttgacctggtgccctggaacagtactttctatactatccagacgtatgtcttccctgtcactacttgcttggcacacagcaatagctgcctcaaccctgtgctgtactgtctcctgaggcgggagccccggcaggctctggcaggcaccttcagggatctgcggttgaggctgtggccccagggcggaggctgggtgcaacaggtggccctaaagcaggtaggcaggcggtgggtcgcaagcaacccccgggagagccgcccttctaccctgctcaccaacctggacagagggacacccgggtga

SEQ ID NO: 2 - DNA, 1125 bp, Homo sapiens
atgcccacactcaatacttctgcctctccacccacattcttctgggccaatgcctccggaggcagtgtgctgagtgctgatgatgctccgatgcctgtcaaattcctagccctgaggctcatggttgccctggcctatgggcttgtgggggccattggcttgctgggaaatttggcggtgctgtgggtactgagtaactgtgcccggagagcccctggcccaccttcagacaccttcgtcttcaacctggctctggcggacctgggactggcactcactctccccttttgggcagccgagtcggcactggactttcactggcccttcggaggtgccctctgcaagatggttctgacggccactgtcctcaacgtctatgccagcatcttcctcatcacagcgctgagcgttgctcgctactgggtggtggccatggctgcggggccaggcacccacctctcactcttctgggcccgaatagccaccctggcagtgtgggcggcggctgccctggtgacggtgcccacagctgtcttcggggtggagggtgaggtgtgtggtgtgcgcctttgcctgctgcgtttccccagcaggtactggctgggggcctaccagctgcagagggtggtgctggctttcatggtgcccttgggcgtcatcaccaccagctacctgctgctgctggccttcctgcagcggcggcaacggcggcggcaggacagcagggtcgtggcccgctctgtccgcatcctggtggcttccttcttcctctgctggtttcccaaccatgtggtcactctctggggtgtcctggtgaagtttgacctggtgccctggaacagtactttctatactatccagacgtatgtcttccctgtcactacttgcttggcacacagcaatagctgcctcaaccctgtgctgtactgtctcctgaggcgggagccccggcaggctctggcaggcaccttcagggatctgcggttgaggctgtggccccagggcggaggctgggtgcaacaggtggccctaaagcaggtaggcaggcggtgggtcgcaagcaacccccgggagagccgcccttctaccctgctcaccaacctggacagagggacacccgggtga

SEQ ID NO: 3 - protein, 375 aa, Homo sapiens
MPTLNTSASPPTFFWANASGGSVLSADDAPMPVKFLALRLMVALAYGLVGAIGLLGNLAVLWVLSNCARRAPGPPSDTFVFNLALADLGLALTLPFWAAESALDFHWPFGGALCKMVLTATVLNVYASIFLITALSVARYWVVAMAAGPGTHLSLFWARIATLAVWAAAALVTVPTAVFGVEGEVCGVRLCLLRFPSRYWLGAYQLQRVVLAFMVPLGVITTSYLLLLAFLQRRQRRRQDSRVVARSVRILVASFFLCWFPNHVVTLWGVLVKFDLVPWNSTFYTIQTYVFPVTTCLAHSNSCLNPVLYCLLRREPRQALAGTFRDLRLRLWPQGGGWVQQVALKQVGRRWVASNPRESRPSTLLTNLDRGTPGZ

SEQ ID NO: 4 - DNA, 1389 bp, Mus musculus
tagaccaacacccagattccaagggctcttctaagagctctcctgagacaacagcggcggcgggtggttgcttgccaggccggaaggcgggcactccctggttcctctgctctgctgtgctctagcaacctccgcggtcttgcgatggccacatccaattcttctgcctctctgcccaccctcttctgggtcaatggctctggagacagcgtgctgagcactgacggtgctgccatgcctgtccagttccttgttctgaggatcatggttgcactggcctatggacttgtaggtatcattggcttgctgggaaatttggccgtactgtgggttctaggtaactgtggtcagcgtgtgcccggcctgtcttctgatacctttgtcttcagcctggctctagcagacttggggctggcccttactctccctttctgggcaaccgagtcagcaatggacttccactggcctttcggaagtgccctctgcaaggtagtcctgaccaccaccgtcctcagcatctatgccagcaccttcctaatcacagcactgagtatcgcgcgatactgggtggtagccatggctgtgggaccaggtagtcacctctcagtcttttgggcccgtgtggtcaccctggcagtgtgggtggcagctgccctggtgactgtgcccacagcaatctttggggctgaagttgagttgtggggcgtgtgcctctgtcttctgcgtttccccagcagatactggctgggagcttaccagctacagagggtagttctggccttcatcgtgcccttgggagtcattaccaccagttacctgctgctgttggcctttctagagcggcagcaaagatgcaggccacgacaatggcaggacagccgagtggtagcccgctctgtccgtgtcctggtggcttccttcgccctctgctgggttcccaaccatgtagtcactctctgggaaattctggtaaggtttgacctggtgccctgggacagtactttctacacctttcatacttacatccttcccatcaccacctgcttggcccacagcaacagctgcctcaaccctgtgatctattgtctcctgcggcgggagccccagcaggttcttgtcagctccttcagagctctctggtcaagactgtggcctcaaaggaaggcctgcatggaacaaatggccctcaaggaggtaggcgggagaacggtagccagcacccaggagagtggctcttctaggacacacacaaacacaatggaacacctggatgaaggatgcagcctgaacactctcctttctgagacctatcaggggcagagcccacagattctagggaggagcagctgctctctcagtcaggctgctgtgtccccaggagaagtctga

SEQ ID NO: 5 - protein, 415 aa, Mus musculus
MATSNSSASLPTLFWVNGSGDSVLSTDGAAMPVQFLVLRIMVALAYGLVGIIGLLGNLAVLWVLGNCGQRVPGLSSDTFVFSLALADLGLALTLPFWATESAMDFHWPFGSALCKVVLTTTVLSIYASTFLITALSIARYWVVAMAVGPGSHLSVFWARVVTLAVWVAAALVTVPTAIFGAEVELWGVCLCLLRFPSRYWLGAYQLQRVVLAFIVPLGVITTSYLLLLAFLERQQRCRPRQWQDSRVVARSVRVLVASFALCWVPNHVVTLWEILVRFDLVPWDSTFYTFHTYILPITTCLAHSNSCLNPVIYCLLRREPQQVLVSSFRALWSRLWPQRKACMEQMALKEVGGRTVASTQESGSSRTHTNTMEHLDEGCSLNTLLSETYQGQSPQILGRSSCSLSQAAVSPGEVZ

SEQ ID NO: 6 - DNA, 27 bp, Artificial Sequence
  Oligonucleotide primer
ttgtgcagagttcaatggagaatgttg

SEQ ID NO: 7 - DNA, 27 bp, Artificial Sequence
  Oligonucleotide primer
ccagaaacactctacgcctgtcacctg

SEQ ID NO: 8 - DNA, 38 bp, Artificial Sequence
  Oligonucleotide primer
tttgcggccgcaaagtgactcatgctgctcccatcttc

SEQ ID NO: 9 - DNA, 35 bp, Artificial Sequence
  Oligonucleotide primer
aaaactagtcccagcaagccaatgatacctacaag

SEQ ID NO: 10 - DNA, 36 bp, Artificial Sequence
  Oligonucleotide primer
tttggcgcgcctgggacagtactttctacacctttc

SEQ ID NO: 11 - DNA, 38 bp, Artificial Sequence
  Oligonucleotide primer
tttggccggcctccatttaagaagagatcttgagccag

SEQ ID NO: 12 - DNA, 27 bp, Artificial Sequence
  Oligonucleotide primer
tggatccttttattttggagactgaac

SEQ ID NO: 13 - DNA, 27 bp, Artificial Sequence
  Oligonucleotide primer
cctggctcaagatctcttcttaaatgg

SEQ ID NO: 14 - DNA, 27 bp, Artificial Sequence
  Oligonucleotide primer
ggtgagcaatcagatcatgagacttac

SEQ ID NO: 15 - DNA, 27 bp, Artificial Sequence
  Oligonucleotide primer
gcttaccagctacagagggtagttctg

SEQ ID NO: 16 - DNA, 29 bp, Artificial Sequence
  Oligonucleotide primer
tgatgggaaggatgtaagtatgaaaggtg

SEQ ID NO: 17 - DNA, 27 bp, Artificial Sequence
  Oligonucleotide primer
gtcgtgacccatggcgatgcctgcttg

SEQ ID NO: 18 - DNA, 27 bp, Artificial Sequence
  Oligonucleotide primer
cggatccactagataacttcgtatagc

SEQ ID NO: 19 - DNA, 7100 bp, Mus musculus
gtgtgtgtttgtgtgcacttatgcatttgtgcagagttcaatggagaatgttgggtgatagtcttcactagccaccttaagatggtcttcttgttcacccataggtgacccatgagtttcccaggtccaactatgtagtggtttaaataagtatttgcccgtaggctcagatatttaaacactgggagttcagttagtgaccctaactgtgctgtttggggaggttggaaacctttgacaggtggagccttgttagaagaatgtctccaggtgacaggcgtagagtgtttctggtcttgcctcacttcctgctctcttagtgccaagctcttgttactgtgcctgctgccctgcctccattcttccccaccatggtggactctagccttcaggaaccataagccgaaagaaacttccttaattaagttgccttggccatggcattttctcagagcaacagaaaagtgtagtgagaattttggtttctttaaaaaccacatgacggccgggcatggtggcgcatgcctttaatcccagcactcgggaggcagaggcaggcggatttctgagtttgaggccagcctggtctacaaagtgagctccaggacagccagggctatacagagaaaccatgtctcgaaaaaacaaacaaacaaacaaaaacaaaaaacaaaaaaaccacagaactaggaatgtgcttctgttttgatcctaggtgtggggacacggggctgcttcagattgagcacagcagctgaccttggcttgccccgtgctctaacaggagtgtgactttcccagttacagatagtttttgtgactgtgtgacaattgggatgctggggacttttcagagggtgtataaatgctagggccccaagagggagcatgggttgttggttgctgtgaattgttgtcagtctccatttgttaagtggttgtgtgcaaagaagaagcaagaaaaagaaattagatatcctgatggcaaagatcaaacttgcctcaaggaactcaatgctcctaagaagggagtggcctaacaataacatcgccccttcccgacccctgaatttacccaggaatctctttcctttcctctctgtccccttttctcttctatctaatgctgcaagaaaagggatggagaagggtggaagaaagaaaaactcacaacgtagcccaagtcctgctacagaaaagtgactcatgctgctcccatcttcctgggagtcccagggttacaggtgctctctgctgagcacagctctgcctgcataagctctaggcctctgaatggggaccctcatgcatgcatcttatctcctcagtaaactccctctccaacctaagctgcatggttttggttttattctgtgtgcaagtgatggggttggaggattgcagcaagcaagggagcagtgttctgctgatctacaccggcagccgggtcgctggagtgcctaagctggctatacaattctggccaaaacagcatggccgtaagaaatgaatacttgacatgagtcggactcagactatgacgtcaggaagacccctccgggcacaagctcaggctgtgaaatctcaccaacccccaccctggaaaactctgccccacaagaaaagctgtataacacgtgtcttctgctcagttctctgtagcttctctccctagctgaggtgtctttcccaataaatctattgtgtggggtttgttgtgccgagtgactttgtgctattattccttgactcctgactgctaggacacctttctcttcacagctataacacaggtagcccttgggttgcagctgagtgccttctgccaacctcatcccacctctctcttgctcaaaaccccaacattctgtttcccagaaagcaactctctcacgtctggggttctcatatgggctagagcttctctgaaatgcctctctgaccccacccccagcaccccaagacaaccgagtaccagatacttccttaaccagcagaagagtgccgaactgctctcagaatccttgaaaaaaaaatctcactcttacctccccagtccatgctctctcccaaagctcctgccgtggagagagatgtgaggagagaaaggagatgatgccattagagacggtcttcagctgagactgagctcatggggagacaggatgaggtgtggtaaaggcttttctcaggttccagaactctacagtgaggcttgggatatggaggaatgtggatatgacggggctctgagtacaccagaaaaatggagtggcagtgggggtgggacaggttggcaggactgttcagggatgtagagcagacctgaaggtgtggccgtggtcatccacctatggcctcacaagtccaatgtgcccagaaggggtatacagggaactgcaacttcctccgtctcatttctttccccttcttagcatcaaggtcacatggtactgcactgaattctgacctgtgttttagttggcagtgttcccttggcatgataacagctgttcattgtcctgcttccttccatgaccaatgctccaaacttccccttgaaatgacagtcatcatgcaaagaacaaggtatggactcacgctagctccctgaacaacggactatcttcatctgatttaacttctgtaaggaatttttatttatatgtatgtctgcacgtatggacatgtagcacattcgagccagtggagaccaaaagagggggtcaaattctttagaactggagtgatagatgacggtgagatactagatgggtgctgggtaccacacgcaggtcatctgcaagatactaactccttaggtatctctagccccaatttaaaactatttaaatagtatttgcctgtgtgagcatggatgttcagtatgctactgaatgcaggcaaatgtcaggacaactttcaggaatctgttctctcctcccaccttgtggatcctgaccacccagtgcacattgtcagggccgacaagcaggtacttgatatctagtcccaaacttgtttggtttggttttgagacagggtttcagaacctaaccttgtctggcctggaattcagtagacagaccaggctgggtgtgccagcaaaccattaagcctgtcaccatctaatcttattttgaagatttagtagtgctggggggtacatactactgtgcatgtataaaagtcagaagacaaccccgggatgcctgtcctctcctacctttatgtaggcaggttccagggacagccatctccacgggcgtatttatttatatatagattataccttgttgtactgccaagtttggccttggtgatcaggtgatccagctgcctcagccatgcaggtagcagtgaccacagctctttggcatctcgctgaattgtatattttcttaacacttttggggggagaattgtgtgtgtgtgtgtgtgtgtgtgtgtgtgtgtgtgtgtgtgagatgagtgtggaggtcaaagggcagcttgtgagtggcatgtctatccttctcctatgcagatcctgtagactgaattcaaatcaccaggcttggcagcaggcccctttccccaaacagccccgatctctggcctgcccccattaacattttaagcaaagcagcacagggccccaatcagagagacacaggaaaggcacagcaactgtgggactctgagtgatgtgtgttcctctctctttttctgactcccagtctggttttctaatgccatattccttccctaacctctcctcacacctctccttttctcagagagctctctgcactccggggagagagaaaaagcgtcttctggtttggctccacatctctactgtttccctttccttccctattacatgcatgttgacagtgtgaacatagccctcccttgcactgccccacccaccacggttgtccaactgggaagaagcaggctgttctacccacacctgtccctcttaggtgtgagcatgggcaggggctcttacaacccgaagctctagaccaacacccagattccaagggctcttctaagagctctcctgagacaacagcggcggcgggtggttgcttgccaggccggaaggcgggcactccctggttcctctgctctgctgtgctctagcaacctccgcggtcttgcgatggccacatccaattcttctgcctctctgcccaccctcttctgggtcaatggctctggagacagcgtgctgagcactgacggtgctgccatgcctgtccagttccttgttctgaggatcatggttgcactggcctatggacttgtaggtatcattggcttgctgggaaatttggccgtactgtgggttctaggtaactgtggtcagcgtgtgcccggcctgtcttctgatacctttgtcttcagcctggctctagcagacttggggctggcccttactctccctttctgggcaaccgagtcagcaatggacttccactggcctttcggaagtgccctctgcaaggtagtcctgaccaccaccgtcctcagcatctatgccagcaccttcctaatcacagcactgagtatcgcgcgatactgggtggtagccatggctgtgggaccaggtagtcacctctcagtcttttgggcccgtgtggtcaccctggcagtgtgggtggcagctgccctggtgactgtgcccacagcaatctttggggctgaagttgagttgtggggcgtgtgcctctgtcttctgcgtttccccagcagatactggctgggagcttaccagctacagagggtagttctggccttcatcgtgcccttgggagtcattaccaccagttacctgctgctgttggcctttctagagcggcagcaaagatgcaggccacgacaatggcaggacagccgagtggtagcccgctctgtccgtgtcctggtggcttccttcgccctctgctgggttcccaaccatgtagtcactctctgggaaattctggtaaggtttgacctggtgccctgggacagtactttctacacctttcatacttacatccttcccatcaccacctgcttggcccacagcaacagctgcctcaaccctgtgatctattgtctcctgcggcgggagccccagcaggttcttgtcagctccttcagagctctctggtcaagactgtggcctcaaaggaaggcctgcatggaacaaatggccctcaaggaggtaggcgggagaacggtagccagcacccaggagagtggctcttctaggacacacacaaacacaatggaacacctggatgaaggatgcagcctgaacactctcctttctgagacctatcaggggcagagcccacagattctagggaggagcagctgctctctcagtcaggctgctgtgtccccaggagaagtctgatctttgatcaccaactctgggtgtgacagaacggagaagctggagtccaaacaggaggtggatgtggcaaagcttatctctggagatggcaaagaggaactgagaataaaccagatcggtcagagactgtcttgacctttcatgcaagtacttcacaggataactaacgtccatcacccggtttagactaacaggtcaggtgtcggttctccctgtcactttagaatagggcacccgttatgcctctttggtaccaaacccaaaaatgtattctctggccaataagctttttgtcatcttagaggtacccattaggaatgatgtagaagcctccccttcaacgtttgtctgtcgttttgctgccacaagatgcagaccctgagtgtatagcctttgagaatagtgaatagatctgtccctttcaatcaaggattgggtaacaatcacaagggtctgggcgtgggggtggggagtcaagagatacagaaaagttttgtaggctgagggtcagaaaccagaagctagtctcactgagtacaactgcacttcagccaagcgccagagcatgtgggctggaatcttccccccacgcaaattattctagaagtcagttctccccttctaaaattggaggcagggtttcatcatacccaggctggtctcacactgtaaagctgagggtcgcttggaatctttaatcttgatcccagatgctcgaattataggcatgtgccaacaagttgagcttttcagatcattttagcctattcttccttctttcctgtacttatgtatgtatgtatgcatgcttgcatgtacatttgtgttcacacttgtacgtgtgtcagaagcaaacataaggttttttcatttcctcgtctttgtttttattgagacggtcttacgacctaaccctaactgtcctggaattcactctgtagaccaggctgtcccttgaactcagacactcacctgcttctgcctcccatgtgctggaactacaggcatgtgccaacgcaccttgctttgatttttaaaaaaattacactttattcaagtgtgttcagacgaggagacatgttccacatgcatgtggcgatcaggacagcttggggtctgctttctctttcccccctcttaagattctgggagtcagcttaggccatcaggctttcgggcaagggtctttacccggggaagcagttttctaaaccctccacacgatgtttatttgggttttcttgtttttttctgagacagggtttctctgtgtagccctggctgtcctggagctcactttgtagaccaagctgacttcgaattcagaaatccgcctgcctctgcctcccaagagctgggattaaaggcgtgctccaccacgcctggctcaagatctcttcttaaatggaataaaaggttcagtctccaaaataaaaggatccagcagtgccataggcagggttcccggtactgacaccttccatggaaaatggaaagacgacagaaaacatgggcagctggcagacaggtacaggtgagccactgaggtctcagtggtatcttacatggacttgtttatggaatataatgtaagtctcatgatctgattgctcaccctcacgcgcgcgcgcacatttgtgagaacacacacacataataaagtaagaaccagcaagatggcgcagcagggaaaacacatggc

SEQ ID NO: 20 - protein, 259 aa, Homo sapiens
GNILVILVILRTKKLRTPTNIFILNLAVADLLFLLTLPPWALYYLVGGSEDWPFGSALCKLVTALDVVNMYASILLLTAISIDRYLAIVHPLRYRRRRTSPRRAKVVILLVWVLALLLSLPPLLFSWVKTVEEGNGTLNVNVTVCLIDFPEESTASVSTWLRSYVLLSTLVGFLLPLLVILVCYTRILRTLRKAAKTLLVVVVVFVLCWLPYFIVLLLDTLCLSIIMSSTCELERVLPTALLVTLWLAYVNSCLNPIIY

SEQ ID NO: 21 - protein, 4 aa, Artificial Sequence
  Consensus sequence
LTLP

SEQ ID NO: 22 - protein, 4 aa, Artificial Sequence
  Consensus sequence
WPFG

SEQ ID NO: 23 - protein, 4 aa, Artificial Sequence
  Consensus sequence
ALCK

SEQ ID NO: 24 - protein, 4 aa, Artificial Sequeces
  Consensus sequence
YASI

SEQ ID NO: 25 - protein, 6 aa, Artificial Sequence
  Consensus sequence
NSCLNP

(19) United States
(12) Patent Application Publication
(10) Pub. No.: US 2010/0311077 A1
(43) Pub. Date: Dec. 9, 2010

(54) Use of GPR100 Receptor in Diabetes and Obesity Regulation

(75) Inventors: Samuel Aparicio, Vancouver (CA); John Dixon, Singapore (SG); Alan Hendrick, Cambridge (GB); Jennifer M. Horwood, Canterbury (GB); Dirk Zahn, Singapore (SG)

Correspondence Address: EDWARDS ANGELL PALMER & DODGE LLP, P.O. BOX 55874, BOSTON, MA, 02205, US

(73) Assignee: Takeda Pharmaceutical Company Limited, Osaka-Shi (JP)

(21) Appl. No.: 12/725,415

(22) Filed: Mar. 16, 2010

Related U.S. Application Data
(63) Continuation of application No. 11/643,408, filed on Dec. 21, 2006, now abandoned.
Application No. 11/643,408 is a continuation-in-part of application No. PCT/GB05/02434, filed on Jun. 21, 2005.
(60) Provisional application No. 60/586,618, filed on Jul. 9, 2004.
(60) Provisional application No. 60/620,854, filed on Oct. 21, 2004.

(30) Foreign Application Priority Data
Jun. 21, 2004 (GB) 0413872.3
Oct. 20, 2004 (GB) 0423327.6

(51) Int. Cl. G01N 33/53

(52) U.S. Cl. 435/7.1

(57) ABSTRACT

We describe a method of identifying a molecule suitable for the treatment, prophylaxis or alleviation of a Gpr100 associated disease, in particular diabetes and obesity, the method comprising determining whether a candidate molecule is an agonist or antagonist of Gpr100 polypeptide, in which the Gpr100 polypeptide comprises the amino acid sequence shown in SEQ ID NO: 3 or SEQ ID NO: 5, or a sequence which is at least 90% identical thereto.

### CROSS-REFERENCE TO RELATED APPLICATIONS

This application is a continuation of U.S. patent application Ser. No. 11/643,408, filed Dec. 21, 2006, which is a continuation-in-part of International Application number PCT/GB2005/002434 filed Jun. 21, 2005, published as WO 2005/124361 on Dec. 29, 2005 which claims the benefit of GB Application Nos. 0413872.3 filed Jun. 21, 2004 and 0423327.6 filed Oct. 20, 2004 and of U.S. Application Nos. 60/586,618 filed Jul. 9, 2004 and 60/620,854 filed Oct. 21, 2004.

The foregoing applications, and each document cited or referenced in each of the present and foregoing applications, including during the prosecution of each of the foregoing applications (“application and article cited documents”), and any manufacturer's instructions or catalogues for any products cited or mentioned in each of the foregoing applications and articles and in any of the application and article cited documents, are hereby incorporated herein by reference. Furthermore, all documents cited in this text, and all documents cited or reference in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior alt.

### FIELD OF THE INVENTION

This invention relates to newly identified nucleic acids, polypeptides encoded by them and to their production and use. More particularly, the nucleic acids and polypeptides of the present invention relate to a G-protein coupled receptor (GPCR), hereinafter referred to as “Gpr100 GPCR”. The invention also relates to inhibiting or activating the action of such nucleic acids and polypeptides.

### BACKGROUND OF THE INVENTION

It is well established that many medically significant biological processes are mediated by proteins participating in signal transduction pathways that involve G-proteins and/or second messengers, for example, cAMP (Lefkowitz, Nature, 1991, 351: 353-354). These proteins are referred to as proteins participating in pathways with G-proteins or “PPG proteins”. Some examples of these proteins include the GPC receptors, such as those for adrenergic agents and dopamine (Kobilka, B. K., et al., Proc. Natl Acad. Sci., USA, 1987, 84: 46-50; Kobilka B. K., et al., Science, 1987, 238: 650-656; Bunzow, J. R., et al., Nature, 1988, 336: 783-787), G-proteins themselves, effector proteins, for example, phospholipase C, adenyl cyclase, and phosphodiesterase, and actuator proteins, for example, protein kinase A and protein kinase C (Simon, M. I., et al., Science, 1991, 252: 802-8).

For example, in one form of signal transduction, the effect of hormone binding is activation of the enzyme adenylate cyclase inside the cell. Enzyme activation by hormones is dependent on the presence of the nucleotide, GTP. GTP also influences hormone binding. A G-protein connects the hormone receptor to adenylate cyclase. G-protein is shown to exchange GTP for bound GDP when activated by a hormone receptor. The GTP carrying form then binds to activated adenylate cyclase. Hydrolysis of GTP to GDP, catalysed by the G-protein itself, returns the G-protein to its basal, inactive form. Thus, the G-protein serves a dual role, as an intermediate that relays the signal from receptor to effector, and as a clock that controls the duration of the signal.

The membrane protein gene superfamily of G-protein coupled receptors (GPCRs) has been characterised as having seven putative transmembrane domains. The domains are believed to represent transmembrane α-helices connected by extracellular or cytoplasmic loops. G-protein coupled receptors include a wide range of biologically active receptors, such as hormone, viral, growth factor and neuroreceptors.

G-protein coupled receptors (also known as 7™ receptors) have been characterised as including these seven conserved hydrophobic stretches of about 20 to 30 amino acids, connecting at least eight divergent hydrophilic loops. The G-protein family of coupled receptors includes dopamine receptors which bind to neuroleptic drugs used for treating psychotic and neurological disorders. Other examples of members of this family include, but are not limited to, calcitonin, adrenergic, endothelin, cAMP, adenosine, muscarinic, acetylcholine, serotonin, histamine, thrombin, kinin, follicle stimulating hormone, opsins, endothelial differentiation gene-1, rhodopsins, odorant, and cytomegalovirus receptors.

Most G-protein coupled receptors have single conserved cysteine residues in each of the first two extracellular loops which form disulphide bonds that are believed to stabilise functional protein structure. The 7 transmembrane regions are designated as TM1, TM2, TM3, TM4, TM5, TM6, and TM7. TM3 has been implicated in signal transduction.

Phosphorylation and lipidation (pamitylation or farnesylation) of cysteine residues can influence signal transduction of some G-protein coupled receptors. Most G-protein coupled receptors contain potential phosphorylation sites within the third cytoplasmic loop and/or the carboxy terminus. For several G-protein coupled receptors, such as the β-adrenoreceptor, phosphorylation by protein kinase A and/or specific receptor kinases mediates receptor desensitization. For some receptors, the ligand binding sites of G-protein coupled receptors are believed to comprise hydrophilic sockets formed by several G-protein coupled receptor transmembrane domains, the sockets being surrounded by hydrophobic residues of the G-protein coupled receptors. The hydrophilic side of each G-protein coupled receptor transmembrane helix is thought to face inward and form a polar ligand binding site. TM3 has been implicated in several G-protein coupled receptors as having a ligand binding site, such as the TM3 aspartate residue. TM5 serines, a TM6 asparagine and TM6 or TM7 phenylalanines or tyrosines are also implicated in ligand binding.

G-protein coupled receptors can be intracellularly coupled by heterotrimeric G-proteins to various intracellular enzymes, ion channels and transporters (see, Johnson et al., Endoc. Rev., 1989, 10: 317-331). Different G-protein α-subunits preferentially stimulate particular effectors to modulate various biological functions in a cell. Phosphorylation of cytoplasmic residues of G-protein coupled receptors has been identified as an important mechanism for the regulation of G-protein coupling of some G-protein coupled receptors. G-protein coupled receptors are found in numerous sites within a mammalian host. Over the past 15 years, nearly 350 therapeutic agents targeting 7 transmembrane (7 TM) receptors have been successfully introduced onto the market.

Thus, G-protein coupled receptors have an established, proven history as therapeutic targets. Clearly there is a need for identification and characterization of further receptors which can play a role in preventing, ameliorating or correcting dysfunctions or diseases, including, but not limiting to obesity including prevention of obesity or weight gain, appetite suppression, lipid metabolism disorders including hyperlipidemia, dyslipoidemia, and hypertriglyceridemia, depression and anxiety, diabetes and related disorders include but are not limited to: Type I diabetes, Type II diabetes, impaired glucose tolerance, insulin resistance syndromes, syndrome X, hyperglycemia, acute pancreatitis, cardiovascular diseases, hypertension, cardiac hypertrophy, and hypercholesterolemia.

### SUMMARY OF THE INVENTION

A method of identifying a molecule suitable for the treatment, prophylaxis or alleviation of a Gpr100 associated disease, in particular diabetes and obesity, the method comprising determining whether a candidate molecule is an agonist or antagonist of Gpr100 polypeptide, in which the Gpr100 polypeptide comprises the amino acid sequence shown in SEQ ID NO: 3 or SEQ ID NO: 5, or a sequence which is at least 90% identical thereto.

Preferably, the Gpr100 polypeptide is encoded by a nucleic acid sequence shown in SEQ ID NO: 1, SEQ ID NO: 2 or SEQ ID NO: 4, or a sequence which is at least 90% identical thereto.

Preferably, the method comprises exposing the candidate molecule to a Gpr100 polypeptide, and detecting a change in intracellular calcium level as a result of such exposure.

Preferably, the method comprises exposing a non-human animal or a portion thereof, preferably a cell, tissue or organ, to a candidate molecule and determining whether a biological parameter of the animal is changed as a result of the contacting.

Preferably, the biological parameter is selected from the group consisting of serum glucose levels, body weight, glucagon levels, fat percentage.

There is provided, according to a 2^{nd} aspect of the present invention, use of a transgenic non-human animal having a functionally disrupted endogenous Gpr100, or an isolated cell or tissue thereof, as a model for glucose regulation or a Gpr100 associated disease, preferably obesity or diabetes.

Preferably, the transgenic non-human animal comprises a functionally disrupted Gpr100 gene, preferably comprising a deletion in a Gpr100 gene or a portion thereof.

Preferably, the transgenic non-human animal displays a change in any one or more of the following phenotypes when compared with a wild type animal: decreased serum glucose levels, increased body weight, higher fat percentage.

Preferably, the transgenic non-human animal is a rodent, preferably a mouse.

We provide, according to a 3^{rd} aspect of the present invention, use of a Gpr100 polypeptide comprising an amino acid sequence shown in SEQ ID NO: 3 or SEQ ID NO: 5, or a sequence which is at least 90% identical thereto, for the identification of an agonist or antagonist thereof for the treatment, prophylaxis of a Gpr100 associated disease, preferably obesity or diabetes.

As a 4^{th} aspect of the present invention, there is provided use of a Gpr100 polynucleotide comprising a nucleic acid sequence shown in SEQ ID NO: 1, SEQ ID NO: 2 or SEQ ID NO: 4, or a sequence which is at least 90% identical thereto; for the identification of an agonist or antagonist thereof for the treatment, prophylaxis of a Gpr100 associated disease, preferably obesity or diabetes.

We provide, according to a 5^{th} aspect of the present invention, use of a non-human animal or a portion thereof, preferably a cell, tissue or organ, in a method of identifying an agonist or antagonist of Gpr100 polypeptide for use in the treatment, prophylaxis or alleviation of a Gpr100 associated disease, preferably diabetes or obesity.

The present invention, in a 6^{th} aspect, provides use of a an agonist or antagonist identified by a method or use according to any preceding Claim for the treatment, prophylaxis or alleviation of a Gpr100 associated disease, preferably obesity or diabetes.

In a 7^{th} aspect of the present invention, there is provided a method of modulating the regulation of glucose, fat metabolism or weight gain in an individual by modulating the activity of a Gpr100 polypeptide in the individual comprising an amino acid sequence shown in SEQ ID NO: 3 or SEQ ID NO: 5, or a sequence which is at least 90% identical thereto.

Preferably, the method comprises administering an agonist or antagonist of Gpr100 to the individual.

According to an 8^{th} aspect of the present invention, we provide a method of treating an individual suffering from a Gpr100 associated disease, the method comprising increasing or decreasing the activity or amount of Gpr100 polypeptide in the individual.

Preferably, the method comprises administering a Gpr100 polypeptide, an agonist of Gpr100 polypeptide or an antagonist of Gpr100 to the individual

We provide, according to a 9^{th} aspect of the invention, a method of diagnosis of a Gpr100 associated disease, the method comprising the steps of: (a) detecting the level or pattern of expression of Gpr100 polypeptide in an animal suffering or suspected to be suffering from such a disease; and (b) comparing the level or pattern of expression with that of a normal animal.

There is provided, in accordance with a 10^{th} aspect of the present invention, a method of diagnosis of a Gpr100 associated disease, the method comprising detecting a change in a biological parameter as set out above in an individual suspected of suffering from that disease.

As an 11^{th} aspect of the invention, we provide a diagnostic kit for susceptibility to a Gpr100 associated disease, preferably obesity or diabetes, comprising any one or more of the following: a Gpr100 polypeptide or part thereof; an antibody against a Gpr100 polypeptide; or a nucleic acid capable of encoding such.

Preferably, the Gpr100 associated disease is selected from the group consisting of obesity or weight gain, appetite suppression, metabolic disorders, diabetes, including Type I diabetes and Type H diseases, and related disorders and weight related disorders, impaired glucose tolerance, insulin resistance syndromes, syndrome X, peripheral neuropathy, diabetic neuropathy, diabetes associated proteinuria, lipid metabolism disorders including hyperglycemia, hyperlipidemia, dyslipidemia, hypertriglyceridemia, acute pancreatitis, cardiovascular diseases, peripheral vascular disease, hypertension, cardiac hypertrophy, ischaemic heart disease, hypercholesterolemia, obesity, and prevention of obesity or weight gain.

### BRIEF DESCRIPTION OF THE DRAWINGS

FIG. 1 is a diagram showing the results of analysis of the human Gpr100 polypeptide (SEQ ID NO: 3) using the HMM structural prediction software of pfam (available at the pfam website maintained by the Sanger Institute).

FIG. 2 is a diagram of the knockout plamsid.

FIG. 3 is a diagram showing an expression profile for human Gpr100 GPCR generated by reverse transcription-polymerase chain reaction (RT-PCR).

FIG. 4 shows histological sections of white adipose tissue of Gpr100 knockout mice and wild type controls.

FIG. 5 shows the body weight of KO (−/−) and wt (+/+) animals on low (LF) and high fat diet (HF).

FIG. 6 shows the body composition of the same animals as in FIG. 5 measured by DEXA.

FIG. 7 shows the glucose tolerance test of the same animals as in FIG. 5.

FIG. 8 is a graph of glucagon levels of wild type animals and Gpr100 knockout animals.

FIG. 9 is a graph showing results from a glucose tolerance test of overnight fasted (16 hours) wild type animals and Gpr100 knockout animals.

FIG. 10 is a graph of glucose levels from overnight fasted (16 hours) wild type animals and Gpr100 knockout animals.

FIG. 11 is a graph showing RIA analysis of glucagon levels in the terminal blood sample of overnight fasted (16 hours) wild type and Gpr100 knockout animals.

FIG. 12 shows the insulin levels at time 0, 60 and 120 minutes post glucose tolerance (GTT) test.

### SEQUENCE LISTINGS

SEQ ID NO: 1 shows the cDNA sequence of human Gpr100. SEQ ID NO: 2 shows an open reading frame derived from SEQ ID NO: 1. SEQ ID NO: 3 shows the amino acid sequence of human Gpr100. SEQ ID NO: 4 shows the open reading frame of a cDNA for Mouse Gpr100. SEQ ID NO: 5 shows the amino acid sequence of Mouse Gpr100. SEQ ID NOs: 6-19 show the vector construct promoters and knockout vector sequences.

### DETAILED DESCRIPTION

### Gpr100GPCR

We describe a G-Protein Coupled Receptor (GPCR), in particular, an orphan G-protein coupled receptor, which we refer to as Gpr100 GPCR, homologues, variants or derivatives thereof, as well as their uses in the treatment, relief or diagnosis of diseases, including Gpr100 associated diseases such as diabetes and obesity. This and other embodiments of the invention will be described in further detail below.

Gpr100 is also known as Gpcr 142 and relaxin-3 receptor-2, and is structurally related to other proteins of the G-protein coupled receptor family, as shown by the results of sequencing the amplified cDNA products encoding human Gpr100. The cDNA sequence of SEQ ID NO: 1 contains an open reading flame (SEQ ID NO: 2, nucleotide numbers 112 to 1039) encoding a polypeptide of 374 amino acids shown in SEQ ID NO: 3, Human Gpr100 is found to map to Homo sapiens chromosome 1q22.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In. Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. 1 by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855, Lars-Inge Larsson “Immunocytochemistry: Theory and Practice”, CRC Press inc., Baca Raton, Fla., 1988, ISBN 0-8493-6078-1, John D. Pound (ed); “Immunochernical Protocols, vol 80”, in the series: “Methods in Molecular Biology”, Humana Press, Totowa, N.J., 1998, ISBN 0-89603-493-3, Handbook of Drug Screening, edited by Ramakrishna Seethala, Prabhavathi B. Fernandes (2001, New York, N.Y., Marcel Dekker, ISBN 0-8247-0562-9); Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3; and The Merck Manual of Dignosis and Therapy (17th Edition, Beers, M. H., and Berkow, R, Eds, ISBN: 0911910107, John Wiley & Sons). Each of these general texts is herein incorporated by reference. Each of these general texts is herein incorporated by reference.

### Expression Profile of Gpr100

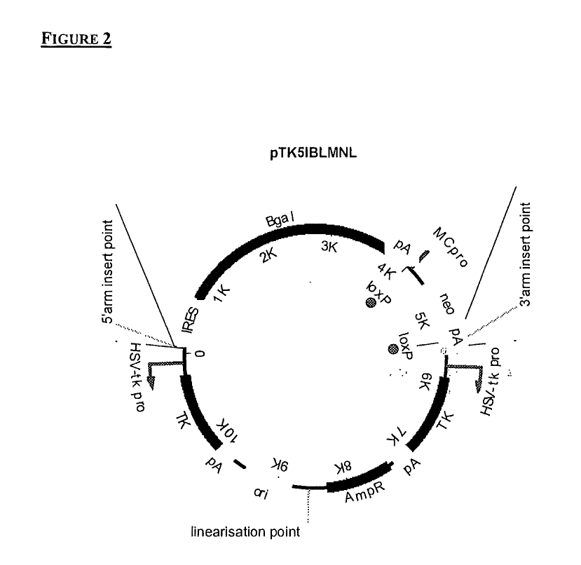

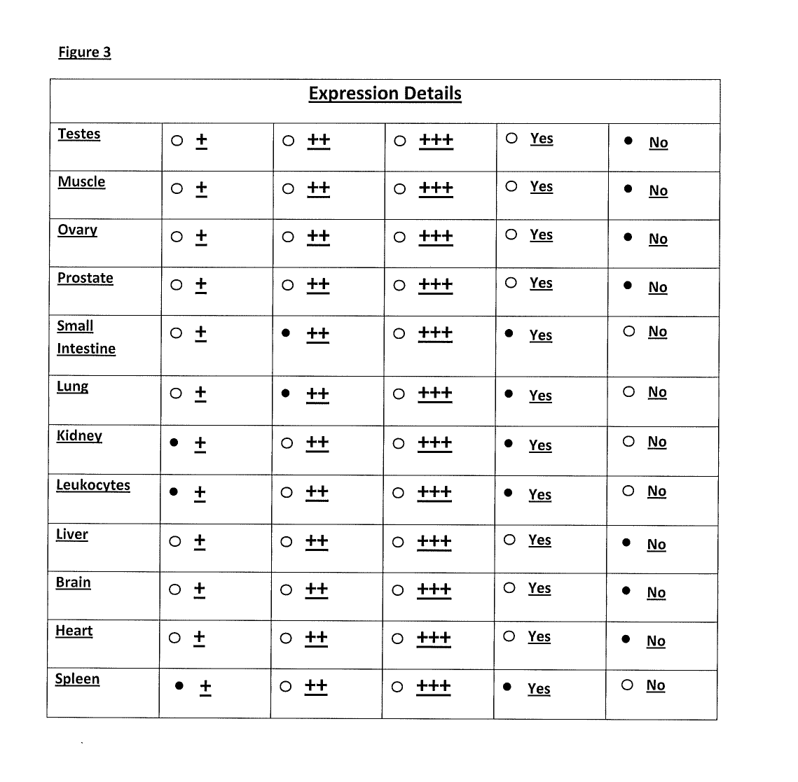

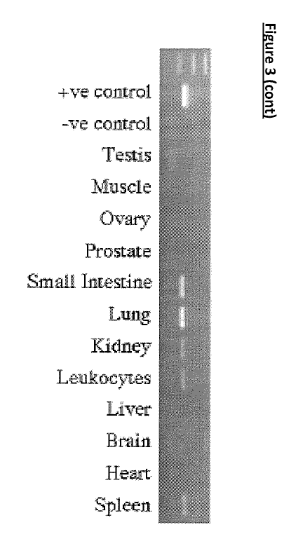

Polymerase chain reaction (PCR) amplification of Gpr100 cDNA detects expression of Gpr100 to varying abundance in small intestine, lung, kidney, leukocytes and spleen. An expression profile of Gpr100 GPCR is shown in FIG. 2. Using Gpr100 cDNA of SEQ ID NO: 1 to search the human EST data sources by BLASTN, identities are found in cDNA derived from libraries originating from bone marrow (BF90022). This indicates that Gpr100 is expressed in these normal or abnormal tissues. Accordingly, the Gpr100 polypeptides, nucleic acids, probes, antibodies, expression vectors and ligands are useful for detection, diagnosis, treatment and other assays for diseases associated with over-, under- and abnormal expression of Gpr100 GPCR in these and other tissues.

This and other embodiments of the invention will be described in further detail below.

### Gpr100 GPCR Associated Diseases

According to the methods and compositions described here, Gpr100 GPCR is useful for treating and diagnosing a range of diseases. These diseases are referred to for convenience as “Gpr100 associated diseases”.

Thus, Gpr100 deficient animals may be used as models for Gpr100 associated diseases. Gpr100, its fragments, homologues, variants and derivatives thereof, as well as modulators, including particularly agonists and antagonists, may be used to diagnose or treat Gpr100 associated diseases. In particular, Gpr100 may be used in a screen for molecules capable of affecting its function, which may be used to treat a Gpr100 associated disease.

We demonstrate here that human Gpr100 maps to Homo sapiens chromosome 1q22. Accordingly, in a specific embodiment, Gpr100 GPCR may be used to treat or diagnose a disease which maps to this locus, chromosomal band, region, arm or the same chromosome.

Known diseases which have been determined as being linked to the same locus, chromosomal band, region, arm or chromosome as the chromosomal location of Gpr100 GPCR (i.e., Homo sapiens chromosome 1q22) include the following (locations in brackets): epilepsy (1q21), Gaucher disease (1q21), lymphoma progression (1q22), Charcot-Marie-Tooth disease, type 1B (1q22), congenital hypomyelinating neuropathy (1q22), and susceptibility to familial combined hyperlipidemia (1q22-q23).

Accordingly, according to a preferred embodiment, Gpr100 GPCR may be used to diagnose or treat, by any means as described in this document epilepsy, Gaucher disease, lymphoma progression, Charcot-Marie-Tooth disease, type 1B, congenital hypomyelinating neuropathy, and susceptibility to familial combined hyperlipidemia.

Knockout mice deficient in Gpr100 display a range of phenotypes, as demonstrated in the Examples

In summary, the experiments described in the Examples reveal the contribution of the Gpr100 receptor to type H diabetes and obesity. Mice deficient in Gpr100 were subjected to procedures including the GTT, the Insulin Suppression Test (IST) and the Glucose-stimulated Insulin Secretion Test (GSIST). Glucose intolerance, as seen in Type II diabetes, can be the result of either insulin insensitivity, which is the inability of muscle, fat or liver cells to take up glucose in response to insulin, or insulin deficiency, usually the result of pancreatic β-cell dysfunction, or both. These measure the ability of the mice to metabolize and/or store glucose, the sensitivity of blood glucose to exogenous insulin, and insulin secretion in response to glucose. These tests are also meant to look at other observables related to diabetes and obesity, such as food intake, metabolic rate, respiratory exchange ratio, activity level, body fat composition, serum chemistry parameters, e.g. leptin, and histology of related organs.

The Examples show that Gpr100 deficient (knockout) animals have a higher body fat content after a long term high fat or an iso-caloric high carbohydrate diet. In addition, the animals develop an impaired glucose tolerance test which is common in animals with increased body fat content.

We conclude that Gpr100 is involved in the regulation of fat and glucose metabolism. Gpr100 deficient animals may therefore be used as models for impaired regulation of glucose and fat metabolism, in particular for diseases such as diabetes and obesity. Gpr100 can be used in assays to screen for compounds useful for the treatment of these diseases.

Furthermore, Example 3 shows that following fasting conditions, homozygous mutant mice exhibited increased white adipose tissue and adipocyte cell size when compared to age and gender matched control mice.

Accordingly, Gpr100 is involved in the regulation of obesity and Gpr100 deficient animals may therefore be used as models for obesity. Gpr100, its fragments, homologues, variants and derivatives thereof, as well as modulators, including particularly agonists and antagonists, may be used to diagnose or treat obesity. In particular, Gpr100 may be used in a screen for molecules capable of affecting its function, which may be used to treat obesity. In general, we disclose a method of decreasing body fat in an individual, preferably for the treatment of obesity, the method comprising increasing the level or activity of Gpr100 in that individual. As noted elsewhere, this can be achieved by up-regulating the expression of Gpr100, or by use of agonists to Gpr100.

Gpr100 Associated Diseases

Thus, Gpr100 associated diseases comprise any of the following: obesity including prevention of obesity or weight gain, appetite suppression, metabolic disorders, diabetes, including Type I diabetes and Type II diseases, and related disorders and weight related disorders, impaired glucose tolerance, insulin resistance syndromes, syndrome X, peripheral neuropathy, diabetic neuropathy, diabetes associated proteinuria, lipid metabolism disorders including hyperglycemia, hyperlipidemia, dyslipidemia, hypertriglyceridemia, acute pancreatitis, cardiovascular diseases, peripheral vascular disease, hypertension, cardiac hypertrophy, ischaemic heart disease, hypercholesterolemia, obesity, and prevention of obesity or weight gain.

As noted above, Gpr100 GPCR may be used to diagnose and/or treat any of these specific diseases using any of the methods and compositions described here. In addition, it was noted that Gpr100 knockouts had suppressed appetites and water intake and therefore compounds capable of modulation Gpr100 function could be used as diet supplements or for dieting and weight loss programmes.

We specifically envisage the use of nucleic acids, vectors comprising Gpr100 GPCR nucleic acids, polypeptides, including homologues, variants or derivatives thereof, pharmaceutical compositions, host cells, and transgenic animals comprising Gpr100 GPCR nucleic acids and/or polypeptides, for the treatment or diagnosis of the specific diseases listed above. Furthermore, we envisage the use of compounds capable of interacting with or binding to Gpr100 GPCR, preferably antagonists of a Gpr100 GPCR, preferably a compound capable of lowering the endogenous level of cyclic AMP in a cell, antibodies against Gpr100 GPCR, as well as methods of making or identifying these, in diagnosis or treatment of the specific diseases and disorders or conditions mentioned above. In particular, we include the use of any of these compounds, compositions, molecules, etc, in the production of vaccines for treatment or prevention of the specific diseases. We also disclose diagnostic kits for the detection of the specific diseases in an individual.

Methods of linkage mapping to identify such or further specific diseases treatable or diagnosable by use of Gpr100 GPCR are known in the art, and are also described elsewhere in this document.

### Glucose Regulation

Glucose is necessary to ensure proper function and survival of all organs. While hypoglycemia produces cell death, chronic hyperglycemia can also result in organ or tissue damage.

Plasma glucose remains in a narrow range, normally between 4 and 7 mM, which is controlled by a balance between glucose absorption from the intestine, production by the liver, and uptake and metabolism by peripheral tissues. In response to elevated plasma levels of glucose, such as after a meal, the beta cells of the pancreatic Islets of Langerhans secrete insulin. Insulin, in turn, acts on muscle and adipose tissues to stimulate glucose uptake into those cells, and on liver cells to inhibit glucose production.

In addition, insulin also stimulates cell growth and differentiation, and promotes the storage of substrates in fat, liver and muscle by stimulating lipogenesis, glycogen and protein synthesis, and inhibiting lipolysis, glycogenolysis and protein breakdown. When plasma levels of glucose decrease, the pancreatic alpha cells secrete glucagon, which in turn stimulates glycolysis in the liver and release of glucose into the bloodstream.

Diabetes and obesity are a diseases which are well known in the art. A summary description of each follows:

### Diabetes

Diabetes is defined as a state in which carbohydrate and lipid metabolism are improperly regulated by insulin. Two major forms of diabetes have been identified, type I and II. Type I diabetes represents the less prevalent form of the disease, affecting 5-10% of diabetic patients. It is thought to result from the autoimmune destruction of the insulin-producing beta cells of the pancreatic Islet of Langerhans. Exogenous administration of insulin typically alleviates the pathophysiology. Type II diabetes is the most common form of the disease and is possibly caused by a combination of defects in the mechanisms of insulin secretion and action. Both forms, type I and type II, have similar complications, but distinct pathophysiology.

The first stage of type II diabetes is characterized by the failure of muscle and/or other organs to respond to normal circulating concentrations of insulin. This is commonly associated with obesity, a sedentary lifestyle, and/or a genetic predisposition. This is followed by an increase in insulin secretion from the pancreatic beta cells, a condition called hyperinsulinemia. Ultimately, the pancreatic beta cells may no longer be able to compensate, leading to impaired glucose tolerance, chronic hyperglycemia, and tissue damage. The complex signaling pathways involved in the regulation of blood glucose and metabolism provide several potential targets for treatment of conditions of abnormal glucose metabolism such as type II diabetes or obesity.

### Obesity

Obesity is a disease that affects at least 39 million Americans: more than one-quarter of all adults and about one in five children. Each year, obesity causes at least 300,000 excess deaths in the U.S. and costs the country more than $100 billion. Over the last 10 years, the proportion of the U.S. population that is obese has increased from 25 percent to 32 percent. Obesity is measured by Body Mass Index, or BMI, which is a mathematical calculation used to determine if a person is obese or overweight. BMI is calculated by dividing a person's body weight in kilograms by their height in meters squared. A BMI of 30 or greater is considered obese, while a BMI of 25-29.9 is considered overweight. However, the criteria for diagnosis can be misleading for people with more muscle mass and less body fat than normal, such as athletes. Over 70 million Americans are considered overweight.

Health problems, including but not limited to cardiovascular disease, blood pressure, Type II diabetes, high cholesterol, gout, certain types of cancer, and osteoarthritis, are associated with overweight conditions and obesity.

### Identities and Similarities to Gpr100

G-protein coupled receptor SALPR somatostatin and angiotensin-like peptide receptor. (Identities=141/322 (43%), Positives=194/322 (59%)).

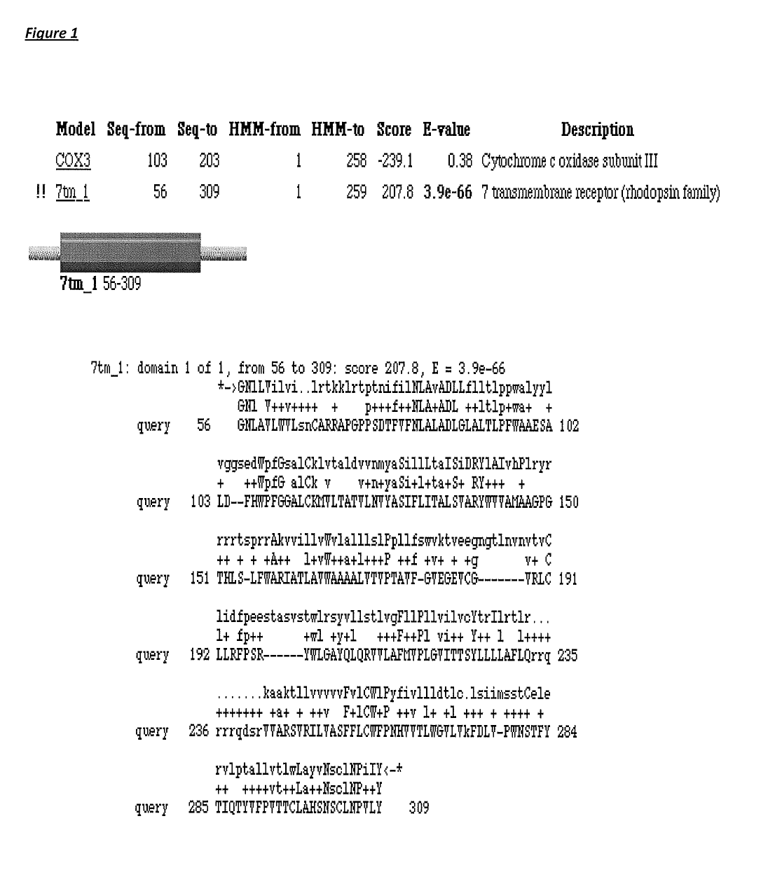

Analysis of the Gpr100 polypeptide (SEQ ID NO: 3) using the HMM structural prediction software of pfam (available at the pfam website maintained by the Sanger Institute) confirms that Gpr100 peptide is a GPCR of the 7™-1 structural class (see FIG. 1).

The mouse homologue of the human Gpr100 GPCR has been cloned, and its nucleic acid sequence and amino acid sequence are shown as SEQ ID NO: 4 and SEQ ID NO: 5 respectively. The mouse Gpr100 GPCR cDNA of SEQ ID NO: 4 shows a high degree of identity with the human Gpr100 GPCR (SEQ ID NO: 2) sequence (Identities=571/693 (82%)), while the amino acid sequence (SEQ ID NO: 5) of mouse Gpr100 GPCR shows a high degree of identity and similarity with human Gpr100 GPCR (SEQ ID NO: 3) (Identities=235/379 (62%), Positives=264/379 (69%)).

Human and mouse Gpr100 GPCR are therefore members of a large family of G Protein Coupled Receptors (GPCRs).

### Gpr100 GPCR Polypeptides

As used here, the term “Gpr100 GPCR polypeptide” is intended to refer to a polypeptide comprising the amino acid sequence shown in SEQ ID NO: 3 or SEQ ID NO: 5, or a homologue, variant or derivative thereof. Preferably, the polypeptide comprises or is a homologue, variant or derivative of the sequence shown in SEQ ID NO: 3.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

“Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications.

Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins—Structure and Molecular Properties, 2nd Ed., T. E. Creighton, W. H. Freeman and Company, New York, 1993 and Wold, F., Posttranslational Protein Modifications: Perspectives and Prospects, pgs. 1-12 in Posttranslational Covalent Modification of Proteins, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Posttranslational Modifications and Aging”, Ann NY Acad Sci (1992) 663:48-62.

The terms “variant”, “homologue”, “derivative” or “fragment” in relation to the present document include any substitution of, variation of, modification of, replacement of, deletion of or addition of one (or more) amino acid from or to a sequence. Unless the context admits otherwise, references to “Gpr100” and “Gpr100 GPCR” include references to such variants, homologues, derivatives and fragments of Gpr100.

Preferably, as applied to Gpr100, the resultant amino acid sequence has GPCR activity, more preferably having at least the same activity of the Gpr100 GPCR shown as SEQ ID NO: 3 or SEQ ID NO: 5. In particular, the term “homologue” covers identity with respect to structure and/or function providing the resultant amino acid sequence has GPCR activity. With respect to sequence identity (i.e. similarity), preferably there is at least 70%, more preferably at least 75%, more preferably at least 85%, even more preferably at least 90% sequence identity. More preferably there is at least 95%, more preferably at least 98%, sequence identity. These terms also encompass polypeptides derived from amino acids which are allelic variations of the Gpr100 GPCR nucleic acid sequence.

Where reference is made to the “receptor activity” or “biological activity” of a receptor such as Gpr100 GPCR, these terms are intended to refer to the metabolic or physiological function of the Gpr100 receptor, including similar activities or improved activities or these activities with decreased undesirable side effects. Also included are antigenic and immunogenic activities of the Gpr100 receptor. Examples of GPCR activity, and methods of assaying and quantifying these activities, are known in the art, and are described in detail elsewhere in this document.

As used herein a “deletion” is defined as a change in either nucleotide or amino acid sequence in which one or more nucleotides or amino acid residues, respectively, are absent. As used herein an “insertion” or “addition” is that change in a nucleotide or amino acid sequence which has resulted in the addition of one or more nucleotides or amino acid residues, respectively, as compared to the naturally occurring substance. As used herein “substitution” results from the replacement of one or more nucleotides or amino acids by different nucleotides or amino acids, respectively.

Gpr100 polypeptides may also have deletions, insertions or substitutions of amino acid residues which produce a silent change and result in a functionally equivalent amino acid sequence. Deliberate amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Gpr100 polypeptides may further comprise heterologous amino acid sequences, typically at the N-terminus or C-terminus, preferably the N-terminus. Heterologous sequences may include sequences that affect intra or extracellular protein targeting (such as leader sequences). Heterologous sequences may also include sequences that increase the immunogenicity of a Gpr100 polypeptide and/or which facilitate identification, extraction and/or purification of the polypeptides. Another heterologous sequence that is particularly preferred is a polyamino acid sequence such as polyhistidine which is preferably N-terminal. A polyhistidine sequence of at least 10 amino acids, preferably at least 17 amino acids but fewer than 50 amino acids is especially preferred.

The Gpr100 GPCR polypeptides may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

Gpr100 polypeptides are advantageously made by recombinant means, using known techniques. However they may also be made by synthetic means using techniques well known to skilled persons such as solid phase synthesis. Gpr100 polypeptides may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences, such as a thrombin cleavage site. Preferably the fusion protein will not hinder the function of the protein of interest sequence.

Gpr100 polypeptides may be in a substantially isolated form. This term is intended to refer to alteration by the hand of man from the natural state. If an “isolated” composition or substance occurs in nature, it has been changed or removed from its original environment, or both. For example, a polynucleotide, nucleic acid or a polypeptide naturally present in a living animal is not “isolated,” but the same polynucleotide, nucleic acid or polypeptide separated from the coexisting materials of its natural state is “isolated”, as the term is employed herein.

It will however be understood that the Gpr100 GPCR protein may be mixed with carriers or diluents which will not interfere with the intended purpose of the protein and still be regarded as substantially isolated. A Gpr100 polypeptide may also be in a substantially purified form, in which case it will generally comprise the protein in a preparation in which more than 90%, for example, 95%, 98% or 99% of the protein in the preparation is a Gpr100 GPCR polypeptide.

The present document also relates to peptides comprising a portion of a Gpr100 polypeptide. Thus, fragments of Gpr100 GPCR and its homologues, variants or derivatives are included. The peptides may be between 2 and 200 amino acids, preferably between 4 and 40 amino acids in length. The peptide may be derived from a Gpr100 GPCR polypeptide as disclosed here, for example by digestion with a suitable enzyme, such as trypsin. Alternatively the peptide, fragment, etc may be made by recombinant means, or synthesised synthetically,

The term “peptide” includes the various synthetic peptide variations known in the art, such as a retroinverso D peptides. The peptide may be an antigenic determinant and/or a T-cell epitope. The peptide may be immunogenic in vivo. Preferably the peptide is capable of inducing neutralising antibodies in vivo.

By aligning Gpr100 GPCR sequences from different species, it is possible to determine which regions of the amino acid sequence are conserved between different species (“homologous regions”), and which regions vary between the different species (“heterologous regions”).

The Gpr100 polypeptides may therefore comprise a sequence which corresponds to at least part of a homologous region. A homologous region shows a high degree of homology between at least two species. For example, the homologous region may show at least 70%, preferably at least 80%, more preferably at least 90%, even more preferably at least 95% identity at the amino acid level using the tests described above. Peptides which comprise a sequence which corresponds to a homologous region may be used in therapeutic strategies as explained in further detail below. Alternatively, the Gpr100 GPCR peptide may comprise a sequence which corresponds to at least part of a heterologous region. A heterologous region shows a low degree of homology between at least two species.

### Gpr100 GPCR Polynucleotides and Nucleic Acids

This disclosure encompasses Gpr100 polynucleotides, Gpr100 nucleotides and Gpr100 nucleic acids, methods of production, uses of these, etc, as described in further detail elsewhere in this document.

The terms “Gpr100 polynucleotide”, “Gpr100 nucleotide” and “Gpr100 nucleic acid” may be used interchangeably, and are intended to refer to a polynucleotide/nucleic acid comprising a nucleic acid sequence as shown in SEQ ID NO: 1, SEQ ID NO: 2 or SEQ ID NO: 4, or a homologue, variant or derivative thereof. Preferably, the polynucleotide/nucleic acid comprises or is a homologue, variant or derivative of the nucleic acid sequence SEQ ID NO: 1 or SEQ ID NO: 2, most preferably, SEQ ID NO: 2.

These terms are also intended to include a nucleic acid sequence capable of encoding a Gpr100 polypeptide and/or a peptide. Thus, Gpr100 GPCR polynucleotides and nucleic acids comprise a nucleotide sequence capable of encoding a polypeptide comprising the amino acid sequence shown in SEQ ID NO: 3 or SEQ ID NO: 5, or a homologue, variant or derivative thereof. Preferably, the Gpr100 GPCR polynucleotides and nucleic acids comprise a nucleotide sequence capable of encoding a polypeptide comprising the amino acid sequence shown in SEQ ID NO: 3, or a homologue, variant or derivative thereof.

“Polynucleotide” generally refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by the skilled person that numerous nucleotide sequences can encode the same polypeptide as a result of the degeneracy of the genetic code.

As used herein, the term “nucleotide sequence” refers to nucleotide sequences, oligonucleotide sequences, polynucleotide sequences and variants, homologues, fragments and derivatives thereof (such as portions thereof). The nucleotide sequence may be DNA or RNA of genomic or synthetic or recombinant origin which may be double-stranded or single-stranded whether representing the sense or antisense strand or combinations thereof. The term nucleotide sequence may be prepared by use of recombinant DNA techniques (for example, recombinant DNA).

Preferably, the term “nucleotide sequence” means DNA.

The terms “variant”, “homologue”, “derivative” or “fragment” in relation to the present document include any substitution of, variation of, modification of, replacement of, deletion of or addition of one (or more) nucleic acids from or to the sequence of a Gpr100 nucleotide sequence. Unless the context admits otherwise, references to “Gpr100” and “Gpr100 GPCR” include references to such variants, homologues, derivatives and fragments of Gpr100.

Preferably, the resultant nucleotide sequence encodes a polypeptide having GPCR activity, preferably having at least the same activity of the GPCR shown as SEQ ID NO: 3 or SEQ ID NO: 5. Preferably, the term “homologue” is intended to cover identity with respect to structure and/or function such that the resultant nucleotide sequence encodes a polypeptide which has GPCR activity. With respect to sequence identity (i.e. similarity), preferably there is at least 70%, more preferably at least 75%, more preferably at least 85%, more preferably at least 90% sequence identity. More preferably there is at least 95%, more preferably at least 98%, sequence identity. These terms also encompass allelic variations of the sequences.

### Calculation of Sequence Homology

Sequence identity with respect to any of the sequences presented here can be determined by a simple “eyeball” comparison (i.e. a strict comparison) of any one or more of the sequences with another sequence to see if that other sequence has, for example, at least 70% sequence identity to the sequence(s).

Relative sequence identity can also be determined by commercially available computer programs that can calculate % identity between two or more sequences using any suitable algorithm for determining identity, using for example default parameters. A typical example of such a computer program is CLUSTAL. Other computer program methods to determine identify and similarity between the two sequences include but are not limited to the GCG program package (Devereux et al 1984 Nucleic Acids Research 12: 387) and FASTA (Atschul et al 1990 J Malec Biol 403-410).

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (Ausubel et al., 1999 ibid, pages 7-58 to 7-60).

Although the final % homology can be measured in terms of identity, the alignment process itself is typically not based on an all-or-nothing pair comparison. Instead, a scaled similarity score matrix is generally used that assigns scores to each pair wise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62.

Advantageously, the BLAST algorithm is employed, with parameters set to default values. The BLAST algorithm is described in detail at the website maintained by the National Center for Biotechnology Information, which is incorporated herein by reference. The search parameters can also be advantageously set to the defined default parameters.

Advantageously, “substantial identity” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (Karlin and Altschul 1990, Proc. Natl. Acad. Sci. USA 87:2264-68; Karlin and Altschul, 1993, Proc. Natl. Acad. Sci. USA 90:5873-7; see the National Center for Biotechnology Information website) with a few enhancements. The BLAST programs are tailored for sequence similarity searching, for example to identify homologues to a query sequence. For a discussion of basic issues in similarity searching of sequence databases, see Altschul et al (1994) Nature Genetics 6:119-129.

The five BLAST programs available at the National Center for Biotechnology Information website perform the following tasks: blastp—compares an amino acid query sequence against a protein sequence database; blastn—compares a nucleotide query sequence against a nucleotide sequence database; blastx—compares the six-frame conceptual translation products of a nucleotide query sequence (both strands) against a protein sequence database; tblastn—compares a protein query sequence against a nucleotide sequence database dynamically translated in all six reading frames (both strands); tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM 120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Clayerie & States (1993) Computers and Chemistry 17:191-201, or, for BLASTN, by the DUST program of Tatusov and Lipman (see the National Center for Biotechnology Information website). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided at the National Center for Biotechnology Information website. In some embodiments, no gap penalties are used when determining sequence identity.

### Hybridisation

The present document also encompasses nucleotide sequences that are capable of hybridising to the sequences presented herein, or any fragment or derivative thereof, or to the complement of any of the above.

Hybridization means a “process by which a strand of nucleic acid joins with a complementary strand through base pairing” (Coombs J (1994) Dictionary of Biotechnology, Stockton Press, New York N.Y.) as well as the process of amplification as carried out in polymerase chain reaction technologies as described in Dieffenbach C W and G S Dveksler (1995, PCR Primer, a Laboratory Manual, Cold Spring Harbor Press, Plainview N.Y.).

Hybridization conditions are based on the melting temperature (Tm) of the nucleic acid binding complex, as taught in Berger and Kimmel (1987, Guide to Molecular Cloning. Techniques, Methods in Enzymology, Vol 152, Academic Press, San Diego Calif.), and confer a defined “stringency” as explained below.

Nucleotide sequences of capable of selectively hybridising to the nucleotide sequences presented herein, or to their complement, will be generally at least 70%, preferably at least 75%, more preferably at least 85 or 90% and even more preferably at least 95% or 98% homologous to the corresponding nucleotide sequences presented herein over a region of at least 20, preferably at least 25 or 30, for instance at least 40, 60 or 100 or more contiguous nucleotides. Preferred nucleotide sequences will comprise regions homologous to SEQ ID NO: 1, 2 or 4, preferably at least 70%, 80% or 90% and more preferably at least 95% homologous to one of the sequences.

The term “selectively hybridizable” means that the nucleotide sequence used as a probe is used under conditions where a target nucleotide sequence is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with P.

Also included within the scope of the present document are nucleotide sequences that are capable of hybridizing to the nucleotide sequences presented herein under conditions of intermediate to maximal stringency. Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related nucleotide sequences.

In a preferred embodiment, we disclose nucleotide sequences that can hybridise to one or more of the Gpr100 GPCR nucleotide sequences under stringent conditions (e.g. 65° C. and 0.1×SSC {1×SSC=0.15 M NaCl, 0.015 M Na₃ Citrate pH 7.0). Where the nucleotide sequence is double-stranded, both strands of the duplex, either individually or in combination, are encompassed by the present disclosure. Where the nucleotide sequence is single-stranded, it is to be understood that the complementary sequence of that nucleotide sequence is also included.

The present disclosure also encompasses nucleotide sequences that are capable of hybridising to the sequences that are complementary to the sequences presented herein, or any fragment or derivative thereof. Likewise, the present disclosure encompasses nucleotide sequences that are complementary to sequences that are capable of hybridising to the relevant sequence. These types of nucleotide sequences are examples of variant nucleotide sequences. In this respect, the term “variant” encompasses sequences that are complementary to sequences that are capable of hydridising to the nucleotide sequences presented herein. Preferably, however, the term “variant” encompasses sequences that are complementary to sequences that are capable of hydridising under stringent conditions (e.g. 65° C. and 0.1×SSC {1×SSC=0.15 M NaCl, 0.015 Na₃ citrate pH 7.0}) to the nucleotide sequences presented herein.

### Cloning of Gpr100 GPCR and Homologues

The present disclosure also encompasses nucleotide sequences that are complementary to the sequences presented here, or any fragment or derivative thereof. If the sequence is complementary to a fragment thereof then that sequence can be used as a probe to identify and clone similar GPCR sequences in other organisms etc.

The present document thus enables the cloning of Gpr100 GPCR, its homologues and other structurally or functionally related genes from human and other species such as mouse, pig, sheep, etc to be accomplished. Polynucleotides which are identical or sufficiently identical to a nucleotide sequence contained in SEQ ID NO: 1, SEQ ID NO: 2, SEQ ID NO: 4 or a fragment thereof, may be used as hybridization probes for cDNA and genomic DNA, to isolate partial or full-length cDNAs and genomic clones encoding Gpr100 GPCR from appropriate libraries. Such probes may also be used to isolate cDNA and genomic clones of other genes (including genes encoding homologues and orthologues from species other than human) that have sequence similarity, preferably high sequence similarity, to the Gpr100 GPCR gene. Hybridization screening, cloning and sequencing techniques are known to those of skill in the art and are described in, for example, Sambrook et al (supra).

Typically nucleotide sequences suitable for use as probes are 70% identical, preferably 80% identical, more preferably 90% identical, even more preferably 95% identical to that of the referent. The probes generally will comprise at least 15 nucleotides. Preferably, such probes will have at least 30 nucleotides and may have at least 50 nucleotides. Particularly preferred probes will range between 150 and 500 nucleotides, more particularly about 300 nucleotides.

In one embodiment, to obtain a polynucleotide encoding a Gpr100 GPCR polypeptide, including homologues and orthologues from species other than human, comprises the steps of screening an appropriate library under stringent hybridization conditions with a labelled probe having the SEQ ID NO: 1, SEQ ID NO: 2, SEQ ID NO: 4 or a fragment thereof and isolating partial or full-length cDNA and genomic clones containing said polynucleotide sequence. Such hybridization techniques are well known to those of skill in the art. Stringent hybridization conditions are as defined above or alternatively conditions under overnight incubation at 42 degrees C. in a solution comprising: 50% formamide, 5×SSC (150 mM NaCl, 15 mM trisodium citrate), 50 mM sodium phosphate (pH7.6), 5×Denhardt's solution, 10% dextran sulphate, and 20 microgram/ml denatured, sheared salmon sperm DNA, followed by washing the filters in 0.1×SSC at about 65 degrees C.

### Functional Assay for Gpr100 GPCR

The cloned putative Gpr100 GPCR polynucleotides may be verified by sequence analysis or functional assays. For example, the putative Gpr100 GPCR or homologue may be assayed for receptor activity as follows. Capped RNA transcripts from linearized plasmid templates encoding the Gpr100 receptor cDNAs are synthesized in vitro with RNA polymerases in accordance with standard procedures. In vitro transcripts are suspended in water at a final concentration of 0.2 mg/ml. Ovarian lobes are removed from adult female toads, Stage V defolliculated oocytes are obtained, and RNA transcripts (10 ng/oocyte) are injected in a 50 nl bolus using a microinjection apparatus. Two electrode voltage clamps are used to measure the currents from individual Xenopus oocytes in response to agonist exposure. Recordings are made in Ca²⁺ free Barth's medium at room temperature. The Xenopus system may also be used to screen known ligands and tissue/cell extracts for activating ligands, as described in further detail below.

### Expression Assays for Gpr100 GPCR

In order to design useful therapeutics for treating Gpr100 GPCR associated diseases, it is useful to determine the expression profile of Gpr100 (whether wild-type or a particular mutant). Thus, methods known in the art may be used to determine the organs, tissues and cell types (as well as the developmental stages) in which Gpr100 is expressed. For example, traditional or “electronic” Not/hems may be conducted. Reverse-transcriptase PCR (RT-PCR) may also be employed to assay expression of the Gpr100 gene or mutant. More sensitive methods for determining the expression profile of Gpr100 include RNAse protection assays, as known in the art.

Northern analysis is a laboratory technique used to detect the presence of a transcript of a gene and involves the hybridization of a labeled nucleotide sequence to a membrane on which RNAs from a particular cell type or tissue have been bound. (Sambrook, supra, ch. 7 and Ausubel, F. M. et al. supra, ch. 4 and 16.) Analogous computer techniques (“electronic Northerns”) applying BLAST may be used to search for identical or related molecules in nucleotide databases such as GenBank or the LIFESEQ database (Incyte Pharmaceuticals). This type of analysis has advantages in that they may be faster than multiple membrane-based hybridizations. In addition, the sensitivity of the computer search can be modified to determine whether any particular match is categorized as exact or homologous.

The polynucleotides and polypeptides including the probes described above, may be employed as research reagents and materials for discovery of treatments and diagnostics to animal and human disease, as explained in further detail elsewhere in this document,

### Expression of Gpr100 GPCR Polypeptides

The disclosure includes a process for producing a Gpr100 GPCR polypeptide. The method comprises in general culturing a host cell comprising a nucleic acid encoding Gpr100 GPCR polypeptide, or a homologue, variant, or derivative thereof, under suitable conditions (i.e., conditions in which the Gpr100 GPCR polypeptide is expressed).

In order to express a biologically active Gpr100 GPCR, the nucleotide sequences encoding Gpr100 GPCR or homologues, variants, or derivatives thereof are inserted into appropriate expression vector, i.e., a vector which contains the necessary elements for the transcription and translation of the inserted coding sequence.

Methods which are well known to those skilled in the art are used to construct expression vectors containing sequences encoding Gpr100 GPCR and appropriate transcriptional and translational control elements. These methods include in vitro recombinant DNA techniques, synthetic techniques, and in vivo genetic recombination. Such techniques are described in Sambrook, J. et al. (1989; Molecular Cloning, A Laboratory Manual, ch. 4, 8, and 16-17, Cold Spring Harbor Press, Plainview, N.Y.) and Ausubel, F. M. et al. (1995 and periodic supplements; Current Protocols in Molecular Biology, ch. 9, 13, and 16, John Wiley & Sons, New York, N.Y.).

A variety of expression vector/host systems may be utilized to contain and express sequences encoding Gpr100 GPCR. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) or tobacco mosaic virus (TMV)) or with bacterial expression vectors (e.g., Ti or pBR322 plasmids); or animal cell systems. This is not limited by the host cell employed.

The “control elements” or “regulatory sequences” are those non-translated regions of the vector (i.e., enhancers, promoters, and 5′ and 3′ untranslated regions) which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, La Jolla, Calif.) or PSPORT1 plasmid (GIBCO/BRL), and the like, may be used. The baculovirus polyhedrin promoter may be used in insect cells. Promoters or enhancers derived from the genomes of plant cells (e.g., heat shock, RUBISCO, and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence encoding Gpr100 GPCR, vectors based on SV40 or EBV may be used with an appropriate selectable marker.

In bacterial systems, a number of expression vectors may be selected depending upon the use intended for Gpr100 GPCR. For example, when large quantities of Gpr100 GPCR are needed for the induction of antibodies, vectors which direct high level expression of fusion proteins that are readily purified may be used. Such vectors include, but are not limited to, multifunctional E. coli cloning and expression vectors such as BLUESCRIPT (Stratagene), in which the sequence encoding Gpr100 GPCR may be ligated into the vector in frame with sequences for the amino-terminal Met and the subsequent 7 residues of β-galactosidase so that a hybrid protein is produced, pIN vectors (Van Heeke, G. and S. M. Schuster (1989) J. Biol. Chem. 264:5503-5509), and the like. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast Saccharomyces cerevisiae, a number of vectors containing constitutive or inducible promoters, such as alpha factor, alcohol oxidase, and PGH, may be used. For reviews, see Ausubel (supra) and Grant et al. (1987; Methods Enzymol. 153:516-544).

In cases where plant expression vectors are used, the expression of sequences encoding Gpr100 GPCR may be driven by any of a number of promoters. For example, viral promoters such as the 35S and 19S promoters of CaMV may be used alone or in combination with the omega leader sequence from TMV. (Takamatsu, N. (1987) EMBO J. 6:307-311.) Alternatively, plant promoters such as the small subunit of RUBISCO or heat shock promoters may be used. (Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. Such techniques are described in a number of generally available reviews. (See, for example, Hobbs, S. or Muny, L. E. in McGraw Hill Yearbook of Science and Technology (1992) McGraw Hill, New York, N.Y.; pp. 191-196.).

An insect system may also be used to express Gpr100 GPCR. For example, in one such system, Autography californica nuclear polyhedrosis virus (AcNPV) is used as a vector to express foreign genes in Spodoptera frugiperda cells or in Trichoplusia larvae. The sequences encoding Gpr100 GPCR may be cloned into a non-essential region of the virus, such as the polyhedrin gene, and placed under control of the polyhedrin promoter. Successful insertion of Gpr100 GPCR will render the polyhedrin gene inactive and produce recombinant virus lacking coat protein. The recombinant viruses may then be used to infect, for example, S. frugiperda cells or Trichoplusia larvae in which Gpr100 GPCR may be expressed. (Engelhard, E. K. et al. (1994) Proc. Nat. Acad. Sci. 91:3224-3227.)

In mammalian host cells, a number of viral-based expression systems may be utilized. In cases where an adenovirus is used as an expression vector, sequences encoding Gpr100 GPCR may be ligated into an adenovirus transcription/translation complex consisting of the late promoter and tripartite leader sequence. Insertion in a non-essential E1 or E3 region of the viral genome may be used to obtain a viable virus which is capable of expressing Gpr100 GPCR in infected host cells. (Logan, J. and T. Shenk (1984) Proc. Natl. Acad. Sci. 81:3655-3659.) In addition, transcription enhancers, such as the Rous sarcoma virus (RSV) enhancer, may be used to increase expression in mammalian host cells.

Thus, for example, the Gpr100 receptors are expressed in either human embryonic kidney 293 (HEK293) cells or adherent dhfr CHO cells. To maximize receptor expression, typically all 5′ and 3′ untranslated regions (UTRs) are removed from the receptor cDNA prior to insertion into a pCDN or pcDNA3 vector. The cells are transfected with individual receptor cDNAs by lipofectin and selected in the presence of 400 mg/ml G418. After 3 weeks of selection, individual clones are picked and expanded for further analysis. HEK293 or CHO cells transfected with the vector alone serve as negative controls. To isolate cell lines stably expressing the individual receptors, about 24 clones are typically selected and analyzed by Northern blot analysis. Receptor mRNAs are generally detectable in about 50% of the G418-resistant clones analyzed.

Human artificial chromosomes (HACs) may also be employed to deliver larger fragments of DNA than can be contained and expressed in a plasmid. HACs of about 6 kb to 10 Mb are constructed and delivered via conventional delivery methods (liposomes, polycationic amino polymers, or vesicles) for therapeutic purposes.

Specific initiation signals may also be used to achieve more efficient translation of sequences encoding Gpr100 GPCR. Such signals include the ATG initiation codon and adjacent sequences. In cases where sequences encoding Gpr100 GPCR and its initiation codon and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers appropriate for the particular cell system used, such as those described in the literature. (Scharf, D. et al. (1994) Results Probl. Cell Differ. 20:125-162.)

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding, and/or function. Different host cells which have specific cellular machinery and characteristic mechanisms for post-translational activities (e.g., CHO, HeLa, MDCK, HEK293, and W138), are available from the American Type Culture Collection (ATCC,. Bethesda, Md.) and may be chosen to ensure the correct modification and processing of the foreign protein.

For long term, high yield production of recombinant proteins, stable expression is preferred. For example, cell lines capable of stably expressing Gpr100 GPCR can be transformed using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase genes (Wigler, M. et al. (1977) Cell 11:223-32) and adenine phosphoribosyltransferase genes (Lowy, I. et al. (1980) Cell 22:817-23), which can be employed in tk⁻ or apr⁻ cells, respectively. Also, antimetabolite, antibiotic, or herbicide resistance can be used as the basis for selection. For example, dhfr confers resistance to methotrexate (Wigler, M. et al. (1980) Proc. Natl. Acad. Sci. 77:3567-70); npt confers resistance to the aminoglycosides neomycin and G-418 (Colbere-Garapin, F. et al (1981) J. Mol. Biol. 150:1-14); and als or pat confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine. (Hartman, S. C. and R. C. Mulligan (1988) Proc. Natl. Acad. Sci. 85:8047-51.) Recently, the use of visible markers has gained popularity with such markers as anthocyanins, β-glucuronidase and its substrate GUS, and luciferase and its substrate luciferin. These markers can be used not only to identify transformants, but also to quantify the amount of transient or stable protein expression attributable to a specific vector system. (Rhodes, C. A. et al. (1995) Methods Mol. Biol. 55:121-131.)

Although the presence/absence of marker gene expression suggests that the gene of interest is also present, the presence and expression of the gene may need to be confirmed. For example, if the sequence encoding Gpr100 GPCR is inserted within a marker gene sequence, transformed cells containing sequences encoding Gpr100 GPCR can be identified by the absence of marker gene function. Alternatively, a marker gene can be placed in tandem with a sequence encoding Gpr100 GPCR under the control of a single promoter. Expression of the marker gene in response to induction or selection usually indicates expression of the tandem gene as well.

Alternatively, host cells which contain the nucleic acid sequence encoding Gpr100 GPCR and express Gpr100 GPCR may be identified by a variety of procedures known to those of skill in the art. These procedures include, but are not limited to, DNA—DNA or DNA-RNA hybridizations and protein bioassay or immunoassay techniques which include membrane, solution, or chip based technologies for the detection and/or quantification of nucleic acid or protein sequences.

The presence of polynucleotide sequences encoding Gpr100 GPCR can be detected by DNA-DNA or DNA-RNA hybridization or amplification using probes or fragments or fragments of polynucleotides encoding Gpr100 GPCR. Nucleic acid amplification based assays involve the use of oligonucleotides or oligomers based on the sequences encoding Gpr100 GPCR to detect transformants containing DNA or RNA encoding Gpr100 GPCR.

A variety of protocols for detecting and measuring the expression of Gpr100 GPCR, using either polyclonal or monoclonal antibodies specific for the protein, are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on Gpr100 GPCR is preferred, but a competitive binding assay may be employed. These and other assays are well described in the art, for example, in Hampton, R. et al. (1990; Serological Methods, a Laboratory Manual, Section IV, APS Press, St Paul, Minn.) and in Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding Gpr100 GPCR include oligolabeling, nick translation, end-labeling, or PCR amplification using a labeled nucleotide. Alternatively, the sequences encoding Gpr100 GPCR, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Mich.), Promega (Madison, Wis.), and U.S. Biochemical Corp. (Cleveland, Ohio). Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding Gpr100 GPCR may be cultured under conditions suitable for the expression and recovery of the protein from cell culture. The protein produced by a transformed cell may be located in the cell membrane, secreted or contained intracellularly depending on the sequence and/or the vector used. M will be understood by those of skill in the art, expression vectors containing polynucleotides which encode Gpr100 GPCR may be designed to contain signal sequences which direct secretion of Gpr100 GPCR through a prokaryotic or eukaryotic cell membrane. Other constructions may be used to join sequences encoding Gpr100 GPCR to nucleotide sequences encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences, such as those specific for Factor XA or enterokinase (Invitrogen, San Diego, Calif.), between the purification domain and the Gpr100 GPCR encoding sequence may be used to facilitate purification. One such expression vector provides for expression of a fusion protein containing Gpr100 GPCR and a nucleic acid encoding 6 histidine residues preceding a thioredoxin or an enterokinase cleavage site. The histidine residues facilitate purification on immobilized metal ion affinity chromatography (IMIAC, described in Porath, J. et al. (1992) Prot. Exp. Purif. 3: 263-281), while the enterokinase cleavage site provides a means for purifying Gpr100 GPCR from the fusion protein. A discussion of vectors which contain fusion proteins is provided in Kroll, D. J. et al. (1993; DNA Cell Biol. 12:441-453).

Fragments of Gpr100 GPCR may be produced not only by recombinant production, but also by direct peptide synthesis using solid-phase techniques. (Merrifield J. (1963) J. Am. Chem. Soc. 85:2149-2154.) Protein synthesis may be performed by manual techniques or by automation. Automated synthesis may be achieved, for example, using the Applied Biosystems 431A peptide synthesizer (Perkin Elmer). Various fragments of Gpr100 GPCR may be synthesized separately and then combined to produce the full length molecule.

### Biosensors

The Gpr100 polypeptides, nucleic acids, probes, antibodies, expression vectors and ligands are useful as (and for the production of) biosensors.

According to Aizawa (1988), Anal. Chem. Symp. 17: 683, a biosensor is defined as being a unique combination of a receptor for molecular recognition, for example a selective layer with immobilized antibodies or receptors such as a Gpr100 G-protein coupled receptor, and a transducer for transmitting the values measured. One group of such biosensors will detect the change which is caused in the optical properties of a surface layer due to the interaction of the receptor with the surrounding medium. Among such techniques may be mentioned especially ellipso-metry and surface plasmon resonance. Biosensors incorporating Gpr100 may be used to detect the presence or level of Gpr100 ligands, for example, nucleotides such as purines or purine analogues, or analogues of these ligands. The construction of such biosensors is well known in the art.

Thus, cell lines expressing Gpr100 receptor may be used as reporter systems for detection of ligands such as ATP via receptor-promoted formation of [3H]inositol phosphates or other second messengers (Watt et al., 1998, J Biol Chem May 29; 273(22):14053-8). Receptor-ligand biosensors are also described in Hoffman et al., 2000, Proc Natl Acad Sci U S A October 10; 97(21):11215-20. Optical and other biosensors comprising Gpr100 may also be used to detect the level or presence of interaction with G-proteins and other proteins, as described by, for example, Figler et al, 1997, Biochemistry December 23; 36(51):16288-99 and Sarrio et al., 2000, Mol Cell Biol 2000 July; 20(14):5164-74). Sensor units for biosensors are described in, for example, U.S. Pat. No. 5,492,840.

### Screening Assays

The Gpr100 GPCR polypeptide, including homologues, variants, and derivatives, whether natural or recombinant, may be employed in a screening process for compounds which bind the receptor and which activate (agonists) or inhibit activation of (antagonists) of Gpr100. Thus, Gpr100 polypeptides may also be used to assess the binding of small molecule substrates and ligands in, for example, cells, cell-free preparations, chemical libraries, and natural product mixtures. These substrates and ligands may be natural substrates and ligands or may be structural or functional mimetics. See Coligan et al., Current Protocols in Immunology 1(2):Chapter 5 (1991).

Gpr100 GPCR polypeptides are responsible for many biological functions, including many pathologies. Accordingly, it is desirous to find compounds and drugs which stimulate Gpr100 GPCR on the one hand and which can inhibit the function of Gpr100 GPCR on the other hand. In general, agonists and antagonists are employed for therapeutic and prophylactic purposes for such conditions as Gpr100 associated diseases.

Rational design of candidate compounds likely to be able to interact with Gpr100 GPCR protein may be based upon structural studies of the molecular shapes of a polypeptide. One means for determining which sites interact with specific other proteins is a physical structure determination, e.g., X-ray crystallography or two-dimensional NMR techniques. These will provide guidance as to which amino acid residues form molecular contact regions. For a detailed description of protein structural determination, see, e.g., Blundell and Johnson (1976) Protein Crystallography, Academic Press, New York.

An alternative to rational design uses a screening procedure which involves in general producing appropriate cells which express the Gpr100 receptor polypeptide on the surface thereof. Such cells include cells from animals, yeast, Drosophila or E. coli. Cells expressing the receptor (or cell membrane containing the expressed receptor) are then contacted with a test compound to observe binding, or stimulation or inhibition of a functional response. For example, Xenopus oocytes may be injected with Gpr100 mRNA or polypeptide, and currents induced by exposure to test compounds measured by use of voltage clamps measured, as described in further detail elsewhere.

Furthermore, microphysiometric assays may be employed to assay Gpr100 receptor activity. Activation of a wide variety of secondary messenger systems results in extrusion of small amounts of acid from a cell. The acid formed is largely as a result of the increased metabolic activity required to fuel the intracellular signalling process. The pH changes in the media surrounding the cell are very small but are detectable by, for example, the CYTOSENSOR microphysiometer (Molecular Devices Ltd., Menlo Park, Calif.). The CYTOSENSOR is thus capable of detecting the activation of a receptor which is coupled to an energy utilizing intracellular signaling pathway such as the Gpr100 G-protein coupled receptor.

Instead of testing each candidate compound individually with the Gpr100 receptor, a library or bank of candidate ligands may advantageously be produced and screened. Thus, for example, a bank of over 200 putative receptor ligands has been assembled for screening. The bank comprises: transmitters, hormones and chemokines known to act via a human seven transmembrane (7™) receptor; naturally occurring compounds which may be putative agonists for a human 7™ receptor, non-mammalian, biologically active peptides for which a mammalian counterpart has not yet been identified; and compounds not found in nature, but which activate 7TM receptors with unknown natural ligands. This bank is used to screen the receptor for known ligands, using both functional (i.e. calcium, cAMP, microphysiometer, oocyte electrophysiology, etc, see elsewhere) as well as binding assays as described in further detail elsewhere. However, a large number of mammalian receptors exist for which there remains, as yet, no cognate activating ligand (agonist) or deactivating ligand (antagonist). Thus, active ligands for these receptors may not be included within the ligands banks as identified to date. Accordingly, the Gpr100 receptor is also functionally screened (using calcium, cAMP, microphysiometer, oocyte electrophysiology, etc., functional screens) against tissue extracts to identify natural ligands. Extracts that produce positive functional responses can be sequentially subfractionated, with the fractions being assayed as described here, until an activating ligand is isolated and identified.

7TM•receptors which are expressed in HEK 293 cells have been shown to be coupled functionally to activation of PLC and calcium mobilization and/or cAMP stimuation or inhibition. One screening technique therefore includes the use of cells which express the Gpr100 GPCR receptor (for example, transfected Xenopus oocytes, CHO or HEK293 cells) in a system which measures extracellular pH or intracellular calcium changes caused by receptor activation. In this technique, compounds may be contacted with cells expressing the Gpr100 receptor polypeptide. A second messenger response, e.g., signal transduction, pH changes, or changes in calcium level, is then measured to determine whether the potential compound activates or inhibits the receptor.

In such experiments, basal calcium levels in the HEK 293 cells in receptor-transfected or vector control cells are observed to be in the normal, 100 nM to 200 nM, range. HEK 293 cells expressing Gpr100 GPCR or recombinant Gpr100 GPCR are loaded with fura 2 and in a single day more than 150 selected ligands or tissue/cell extracts are evaluated for agonist induced calcium mobilization. Similarly, HEK 293 cells expressing Gpr100 GPCR or recombinant Gpr100 GPCR are evaluated for the stimulation or inhibition of cAMP production using standard cAMP quantitation assays. Agonists presenting a calcium transient or cAMP fluctuation are tested in vector control cells to determine if the response is unique to the transfected cells expressing receptor.

Another method involves screening for receptor inhibitors by determining inhibition or stimulation of Gpr100 receptor-mediated cAMP and/or adenylate cyclase accumulation. Such a method involves transfecting a eukaryotic cell with the Gpr100 receptor to express the receptor on the cell surface. The cell is then exposed to potential antagonists in the presence of the recepto. The amount of cAMP accumulation is then measured. If the potential antagonist binds the receptor, and thus inhibits receptor binding, the levels of receptor-mediated cAMP, or adenylate cyclase, activity will be reduced or increased.

In a preferred embodiment the screen employs detection of a change in intracellular calcium concentrations to screen for agonists and antagonists of Gpr100. Specifically we disclose a method in which antagonists of Gpr100 reduce, lower or block ligand induced intracellular calcium release, preferably of a suitably transfected cell. Preferably, the level of intracellular calcium increase is reduced by 10%, 20%, 30%, 40%, 50%, 60%, 70% or more in the presence of an antagonist of Gpr100. Preferably, the intracellular calcium release is lowered by 1 mM, 2 mM, 3 mM, 4 mM, 5 mM, 10 mM, 15 mM, 25 mM, 35 mM, 45 mM, 60 mM, 70 mM or more in the presence of an antagonist of Gpr100.

We further disclose a method in which agonists of Gpr100 increase the intracellular calcium concentration of a suitably transfected cell. Preferably, the conductance is increased by 10%, 20%, 30%, 40%, 50%, 60%, 70% or more in the presence of an agonist of Gpr100. Preferably, the conductance is increased by 1 mM, 2 mM, 3 mM, 4 mM, 5 mM, 10 mM, 15 mM, 25 mM, 35 mM, 45 mM, 60 mM, 70 mM or more in the presence of an agonist of Gpr100.

Another method for detecting agonists or antagonists for the Gpr100 receptor is the yeast based technology as described in U.S. Pat. No. 5,482,835, incorporated by reference herein.

Where the candidate compounds are proteins, in particular antibodies or peptides, libraries of candidate compounds may be screened using phage display techniques. Phage display is a protocol of molecular screening which utilises recombinant bacteriophage. The technology involves transforming bacteriophage with a gene that encodes one compound from the library of candidate compounds, such that each phage or phagemid expresses a particular candidate compound. The transformed bacteriophage (which preferably is tethered to a solid support) expresses the appropriate candidate compound and displays it on their phage coat. Specific candidate compounds which are capable of binding to a Gpr100 polypeptide or peptide are enriched by selection strategies based on affinity interaction. The successful candidate agents are then characterised. Phage display has advantages over standard affinity ligand screening technologies. The phage surface displays the candidate agent in a three dimensional configuration, more closely resembling its naturally occurring conformation. This allows for more specific and higher affinity binding for screening purposes.

Another method of screening a library of compounds utilises eukaryotic or prokaryotic host cells which are stably transformed with recombinant DNA molecules expressing a library of compounds. Such cells, either in viable or fixed form, can be used for standard binding-partner assays. See also Parce et al. (1989) Science 246:243-247; and Owicki et al. (1990) Proc. Nat'l Acad. Sci. USA 87; 4007-4011, which describe sensitive methods to detect cellular responses. Competitive assays are particularly useful, where the cells expressing the library of compounds are contacted or incubated with a labelled antibody known to bind to a Gpr100 polypeptide, such as ¹²⁵I-antibody, and a test sample such as a candidate compound whose binding affinity to the binding composition is being measured. The bound and free labelled binding partners for the polypeptide are then separated to assess the degree of binding. The amount of test sample bound is inversely proportional to the amount of labelled antibody binding to the polypeptide.

Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

Still another approach is to use solubilized, unpurified or solubilized purified polypeptide or peptides, for example extracted from transformed eukaryotic or prokaryotic host cells. This allows for a “molecular” binding assay with the advantages of increased specificity, the ability to automate, and high drug test throughput.

Another technique for candidate compound screening involves an approach which provides high throughput screening for new compounds having suitable binding affinity, e.g., to a Gpr100 polypeptide, and is described in detail in International Patent application no. WO 84/03564 (Commonwealth Serum Labs.), published on Sep. 13, 1984. First, large numbers of different small peptide test compounds are synthesized on a solid substrate, e.g., plastic pins or some other appropriate surface; see Fodor et al. (1991). Then all the pins are reacted with solubilized Gpr100 polypeptide and washed. The next step involves detecting bound polypeptide. Compounds which interact specifically with the polypeptide will thus be identified.

Ligand binding assays provide a direct method for ascertaining receptor pharmacology and are adaptable to a high throughput format. The purified ligand for a receptor may be radiolabeled to high specific activity (50-2000 Ci/mmol) for binding studies. A determination is then made that the process of radiolabeling does not diminish the activity of the ligand towards its receptor. Assay conditions for buffers, ions, pH and other modulators such as nucleotides are optimized to establish a workable signal to noise ratio for both membrane and whole cell receptor sources. For these assays, specific receptor binding is defined as total associated radioactivity minus the radioactivity measured in the presence of an excess of unlabeled competing ligand. Where possible, more than one competing ligand is used to define residual nonspecific binding.

The assays may simply test binding of a candidate compound wherein adherence to the cells bearing the receptor is detected by means of a label directly or indirectly associated with the candidate compound or in an assay involving competition with a labeled competitor. Further, these assays may test whether the candidate compound results in a signal generated by activation of the receptor, using detection systems appropriate to the cells bearing the receptor at their surfaces. Inhibitors of activation are generally assayed in the presence of a known agonist and the effect on activation by the agonist by the presence of the candidate compound is observed.

Further, the assays may simply comprise the steps of mixing a candidate compound with a solution containing a Gpr100 GPCR polypeptide to form a mixture, measuring Gpr100 GPCR activity in the mixture, and comparing the Gpr100 GPCR activity of the mixture to a standard.

The Gpr100 GPCR cDNA, protein and antibodies to the protein may also be used to configure assays for detecting the effect of added compounds on the production of Gpr100 GPCR mRNA and protein in cells. For example, an ELISA may be constructed for measuring secreted or cell associated levels of Gpr100 GPCR protein using monoclonal and polyclonal antibodies by standard methods known in the art, and this can be used to discover agents which may inhibit or enhance the production of Gpr100 GPCR (also called antagonist or agonist, respectively) from suitably manipulated cells or tissues. Standard methods for conducting screening assays are well understood in the art.

Examples of potential Gpr100 GPCR antagonists include antibodies or, in some cases, nucleotides and their analogues, including purines and purine analogues, oligonucleotides or proteins which are closely related to the ligand of the Gpr100 GPCR, e.g., a fragment of the ligand, or small molecules which bind to the receptor but do not elicit a response, so that the activity of the receptor is prevented.

The document therefore also provides a compound capable of binding specifically to a Gpr100 polypeptide and/or peptide.

The term “compound” refers to a chemical compound (naturally occurring or synthesised), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. Preferably the compound is an antibody.

The materials necessary for such screening to be conducted may be packaged into a screening kit. Such a screening kit is useful for identifying agonists, antagonists, ligands, receptors, substrates, enzymes, etc. for Gpr100 GPCR polypeptides or compounds which decrease or enhance the production of Gpr100 GPCR polypeptides. The screening kit comprises: (a) a Gpr100 GPCR polypeptide; (b) a recombinant cell expressing a Gpr100 GPCR polypeptide; (c) a cell membrane expressing a Gpr100 GPCR polypeptide; or (d) antibody to a Gpr100 GPCR polypeptide. The screening kit may optionally comprise instructions for use.

### Transgenic Animals

The present document further encompasses transgenic animals capable of expressing natural or recombinant Gpr100 GPCR, or a homologue, variant or derivative, at elevated or reduced levels compared to the normal expression level. Included are transgenic animals (“Gpr100 knockout”s) which do not express functional Gpr100 receptor as a result of one or more loss of function mutations, including a deletion, of the Gpr100 gene. Preferably, such a transgenic animal is a non-human mammal, such as a pig, a sheep or a rodent. Most preferably the transgenic animal is a mouse or a rat. Such transgenic animals may be used in screening procedures to identify agonists and/or antagonists of Gpr100 GPCR, as well as to test for their efficacy as treatments for diseases in vivo.

For example, transgenic animals that have been engineered to be deficient in the production of Gpr100 GPCR may be used in assays to identify agonists and/or antagonists of Gpr100 GPCR. One assay is designed to evaluate a potential drug (a candidate ligand or compound) to determine if it produces side effects in the absence of Gpr100 GPCR receptors. This may be accomplished by administering the drug to a transgenic animal as discussed above, and then assaying the animal for a particular response. Although any physiological parameter could be measured in this assay, preferred responses include one or more of the following: changes to disease resistance; altered inflammatory responses; altered tumour susceptibility: a change in blood pressure; neovascularization; a change in eating behaviour; a change in body weight; a change in bone density; a change in body temperature; insulin secretion; gonadotropin secretion; nasal and bronchial secretion; vasoconstriction; loss of memory; anxiety; hyporeflexia or hyperreflexia; pain or stress responses.

Tissues derived from the Gpr100 knockout animals may be used in receptor binding assays to determine whether the potential drug (a candidate ligand or compound) binds to the Gpr100 receptor. Such assays can be conducted by obtaining a first receptor preparation from the transgenic animal engineered to be deficient in Gpr100 receptor production and a second receptor preparation from a source known to bind any identified Gpr100 ligands or compounds. In general, the first and second receptor preparations will be similar in all respects except for the source from which they are obtained. For example, if brain tissue from a transgenic animal (such as described above and below) is used in an assay, comparable brain tissue from a normal (wild type) animal is used as the source of the second receptor preparation. Each of the receptor preparations is incubated with a ligand known to bind to Gpr100 receptors, both alone and in the presence of the candidate ligand or compound. Preferably, the candidate ligand or compound will be examined at several different concentrations.

The extent to which binding by the known ligand is displaced by the test compound is determined for both the first and second receptor preparations. Tissues derived from transgenic animals may be used in assays directly or the tissues may be processed to isolate membranes or membrane proteins, which are themselves used in the assays. A preferred transgenic animal is the mouse. The ligand may be labeled using any means compatible with binding assays. This would include, without limitation, radioactive, enzymatic, fluorescent or chemiluminescent labeling (as well as other labelling techniques as described in further detail above).

Furthermore, antagonists of Gpr100 GPCR receptor may be identified by administering candidate compounds, etc, to wild type animals expressing functional Gpr100, and animals identified which exhibit any of the phenotypic characteristics associated with reduced or abolished expression of Gpr100 receptor function.

Detailed methods for generating non-human transgenic animal are described in further detail below. Transgenic gene constructs can be introduced into the germ line of an animal to make a transgenic mammal. For example, one or several copies of the construct may be incorporated into the genome of a mammalian embryo by standard transgenic techniques.

In an exemplary embodiment, the transgenic non-human animals are produced by introducing transgenes into the germline of the non-human animal. Embryonal target cells at various developmental stages can be used to introduce transgenes. Different methods are used depending on the stage of development of the embryonal target cell. The specific line(s) of any animal used are selected for general good health, good embryo yields, good pronuclear visibility in the embryo, and good reproductive fitness. In addition, the haplotype is a significant factor.

Introduction of the transgene into the embryo can be accomplished by any means known in the art such as, for example, microinjection, electroporation, or lipofection. For example, the Gpr100 receptor transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg may be incubated in vitro for varying amounts of time, or reimplanted into the surrogate host, or both. In vitro incubation to maturity is included. One common method in to incubate the embryos in vitro for about 1-7 days, depending on the species, and then reimplant them into the surrogate host.

The progeny of the transgenically manipulated embryos can be tested for the presence of the construct by Southern blot analysis of the segment of tissue. If one or more copies of the exogenous cloned construct remains stably integrated into the genome of such transgenic embryos, it is possible to establish permanent transgenic mammal lines carrying the transgenically added construct.

The litters of transgenically altered mammals can be assayed after birth for the incorporation of the construct into the genome of the offspring. Preferably, this assay is accomplished by hybridizing a probe corresponding to the DNA sequence coding for the desired recombinant protein product or a segment thereof onto chromosomal material from the progeny. Those mammalian progeny found to contain at least one copy of the construct in their genome are grown to maturity.

For the purposes of this document a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. There will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present description will include exogenous genetic material. As set out above, the exogenous genetic material will, in certain embodiments, be a DNA sequence which results in the production of a Gpr100 GPCR receptor. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMKO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

We also provide non-human transgenic animals, where the transgenic animal is characterized by having an altered Gpr100 gene, preferably as described above, as models for Gpr100 receptor function. Alterations to the gene include deletions or other loss of function mutations, introduction of an exogenous gene having a nucleotide sequence with targeted or random mutations, introduction of an exogenous gene from another species, or a combination thereof. The transgenic animals may be either homozygous or heterozygous for the alteration. The animals and cells derived therefrom are useful for screening biologically active agents that may modulate Gpr100receptor function. The screening methods are of particular use for determining the specificity and action of potential therapies for Obesity in particular appetite suppression, lipid metabolism. The animals are useful as a model to investigate the role of Gpr100 receptors in normal brain, heart, spleen and liver function.

Another aspect pertains to a transgenic nonhuman animal having a functionally disrupted endogenous Gpr100 gene but which also carries in its genome, and expresses, a transgene encoding a heterologous Gpr100 protein (i.e., a Gpr100 from another species). Preferably, the animal is a mouse and the heterologous Gpr100 is a human Gpr100. An animal, or cell lines derived from such an animal, which has been reconstituted with human Gpr100, can be used to identify agents that inhibit human Gpr100 in vivo and in vitro. For example, a stimulus that induces signalling through human Gpr100 can be administered to the animal, or cell line, in the presence and absence of an agent to be tested and the response in the animal, or cell line, can be measured. An agent that inhibits human Gpr100 in vivo or in vitro can be identified based upon a decreased response in the presence of the agent compared to the response in the absence of the agent.

The present disclosure also provides for a Gpr100 GPCR deficient transgenic non-human animal (a “Gpr100 GPCR knock-out”). Such an animal is one which expresses lowered or no Gpr100 GPCR activity, preferably as a result of an endogenous Gpr100 GPCR genomic sequence being disrupted or deleted. Preferably, such an animal expresses no GPCR activity. More preferably, the animal expresses no activity of the Gpr100 GPCR shown as SEQ ID NO: 3 or SEQ ID NO: 5. Gpr100 GPCR knock-outs may be generated by various means known in the art, as described in further detail below.

The present disclosure also pertains to a nucleic acid construct for functionally disrupting a Gpr100 gene in a host cell. The nucleic acid construct comprises: a) a non-homologous replacement portion; b) a first homology region located upstream of the non-homologous replacement portion, the first homology region having a nucleotide sequence with substantial identity to a first Gpr100 gene sequence; and c) a second homology region located downstream of the non-homologous replacement portion, the second homology region having a nucleotide sequence with substantial identity to a second Gpr100 gene sequence, the second Gpr100 gene sequence having a location downstream of the first Gpr100 gene sequence in a naturally occurring endogenous Gpr100 gene. Additionally, the first and second homology regions are of sufficient length for homologous recombination between the nucleic acid construct and an endogenous Gpr100 gene in a host cell when the nucleic acid molecule is introduced into the host cell. In a preferred embodiment, the non-homologous replacement portion comprises an expression reporter, preferably including lacZ and a positive selection expression cassette, preferably including a neomycin phosphotransferase gene operatively linked to a regulatory element(s).

Preferably, the first and second Gpr100 gene sequences are derived from SEQ ID NO: 1, SEQ ID NO: 2 or SEQ ID NO: 4, or a homologue, variant or derivative thereof.

Another aspect of the present disclosure pertains to recombinant vectors into which the nucleic acid construct has been incorporated. Yet another aspect pertains to host cells into which the nucleic acid construct has been introduced to thereby allow homologous recombination between the nucleic acid construct and an endogenous Gpr100 gene of the host cell, resulting in functional disruption of the endogenous Gpr100 gene. The host cell can be a mammalian cell that normally expresses Gpr100 from the liver, brain, spleen or heart, or a pluripotent cell, such as a mouse embryonic stem cell. Further development of an embryonic stem cell into which the nucleic acid construct has been introduced and homologously recombined with the endogenous Gpr100 gene produces a transgenic nonhuman animal having cells that are descendant from the embryonic stem cell and thus early the Gpr100 gene disruption in their genome. Animals that carry the Gpr100 gene disruption in their germline can then be selected and bred to produce animals having the Gpr100 gene disruption in all somatic and germ cells. Such mice can then be bred to homozygosity for the Gpr100 gene disruption.

In vitro systems may be designed to identify compounds capable of binding the Gpr100 receptor gene products. Such compounds may include, but are not limited to, peptides made of D- and/or L-BR BR configuration amino acids (in, for example, the form of random peptide libraries, phosphopeptides (in, for example, the form of random or partially degenerate, directed phosphopeptide libraries, antibodies, and small organic or inorganic molecules. Compounds identified may be useful, for example, in modulating the activity of Gpr100 receptor gene proteins, preferably mutant Gpr100 receptor gene proteins; elaborating the biological function of the Gpr100 receptor gene protein; or screening for compounds that disrupt normal Gpr100 receptor gene interactions or themselves disrupt such interactions.

Compounds that are shown to bind to a particular Gpr100 receptor gene product can be further tested for their ability to elicit a biochemical response from the Gpr100 receptor gene protein. Agonists, antagonists AND/OR inhibitors of the expression product can be identified utilizing assays well known in the art.

### Antibodies

For the purposes of this document, the term “antibody”, unless specified to the contrary, includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and F(ab′)ₐ fragments, as well as single chain antibodies (scFv), fusion proteins and other synthetic proteins which comprise the antigen-binding site of the antibody. The antibodies and fragments thereof may be humanised antibodies, for example as described in EP-A-239400. Furthermore, antibodies with fully human variable regions (or their fragments), for example, as described in U.S. Pat. Nos. 5,545,807 and 6,075,181 may also be used. Neutralizing antibodies, i.e., those which inhibit biological activity of the substance amino acid sequences, are especially preferred for diagnostics and therapeutics.

Antibodies may be produced by standard techniques, such as by immunisation or by using a phage display library.

A Gpr100 polypeptide or peptide may be used to develop an antibody by known techniques. Such an antibody may be capable of binding specifically to the Gpr100 GPCR protein or homologue, fragment, etc.

If polyclonal antibodies are desired, a selected mammal (e.g., mouse, rabbit, goat, horse, etc.) may be immunised with an immunogenic composition comprising a Gpr100 polypeptide or peptide Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance amino acid sequence is administered to immunologically compromised individuals for the purpose of stimulating systemic defence.

Serum from the immunised animal is collected and treated according to known procedures. If serum containing polyclonal antibodies to an epitope obtainable from a Gpr100 polypeptide contains antibodies to other antigens, the polyclonal antibodies can be purified by immunoaffinity chromatography. Techniques for producing and processing polyclonal antisera are known in the art. In order that such antibodies may be made, the disclosure also provides Gpr100 amino acid sequences or fragments thereof haptenised to another amino acid sequence for use as immunogens in animals or humans.

Monoclonal antibodies directed against epitopes obtainable from a Gpr100 polypeptide or peptide can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies may be prepared using any technique which provides for the production of antibody molecules by continuous cell lines in culture. These include, but are not limited to, the hybridoma technique originally described by Koehler and Milstein (1975 Nature 256:495-497), the trioma technique, the human B-cell hybridoma technique (Kosbor et al (1983) Immunol Today 4:72; Cote et al (1983) Proc Natl Acad Sci 80:2026-2030) and the EBV-hybridoma technique (Cole et al., Monoclonal Antibodies and Cancer Therapy, pp. 77-96, Alan R. Liss, Inc., 1985).

In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production, of single chain antibodies (U.S. Pat. No. 4,946,779) can be adapted to produce the substance specific single chain antibodies.

Antibodies, both monoclonal and polyclonal, which are directed against epitopes obtainable from a Gpr100 polypeptide or peptide are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the substance and/or agent against which protection is desired. Techniques for raising anti-idiotype antibodies are known in the art. These anti-idiotype antibodies may also be useful in therapy.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments which contain specific binding sites for the polypeptide or peptide may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse W D et al (1989) Science 256:1275-128 1).

Techniques for the production of single chain antibodies (U.S. Pat. No. 4,946,778) can also be adapted to produce single chain antibodies to Gpr100 polypeptides. Also, transgenic mice, or other organisms including other mammals, may be used to express humanized antibodies.

The above-described antibodies may be employed to isolate or to identify clones expressing the polypeptide or to purify the polypeptides by affinity chromatography.

Antibodies against Gpr100 GPCR polypeptides may also be employed to treat Gpr100 associated diseases.

### Diagnostic Assays

This disclosure also relates to the use of Gpr100 GPCR polynucleotides and polypeptides (as well as homologues, variants and derivatives thereof) for use in diagnosis as diagnostic reagents or in genetic analysis. Nucleic acids complementary to or capable of hybridising to Gpr100 GPCR nucleic acids (including homologues, variants and derivatives), as well as antibodies against Gpr100 polypeptides are also useful in such assays.

Detection of a mutated form of the Gpr100 GPCR gene associated with a dysfunction will provide a diagnostic tool that can add to or define a diagnosis of a disease or susceptibility to a disease which results from under-expression, over-expression or altered expression of Gpr100 GPCR. Individuals carrying mutations in the Gpr100 GPCR gene (including control sequences) may be detected at the DNA level by a variety of techniques.

For example, DNA may be isolated from a patient and the DNA polymorphism pattern of Gpr100 determined. The identified pattern is compared to controls of patients known to be suffering from a disease associated with over-, under- or abnormal expression of Gpr100. Patients expressing a genetic polymorphism pattern associated with Gpr100 associated disease may then be identified. Genetic analysis of the Gpr100 GPCR gene may be conducted by any technique known in the art. For example, individuals may be screened by determining DNA sequence of a Gpr100 allele, by RFLP or SNP analysis, etc. Patients may be identified as having a genetic predisposition for a disease associated with the over-, under-, or abnormal expression of Gpr100 by detecting the presence of a DNA polymorphism in the gene sequence for Gpr100 or any sequence controlling its expression.

Patients so identified can then be treated to prevent the occurrence of Gpr100 associated disease, or more aggressively in the early stages of Gpr100 associated disease to prevent the further occurrence or development of the disease.

The present disclosure further discloses a kit for the identification of a patient's genetic polymorphism pattern associated with Gpr100 associated disease. The kit includes DNA sample collecting means and means for determining a genetic polymorphism pattern, which is then compared to control samples to determine a patient's susceptibility to Gpr100 associated disease. Kits for diagnosis of a Gpr100 associated disease comprising Gpr100 polypeptide and/or an antibody against such a polypeptide (or fragment of it) are also provided.

Nucleic acids for diagnosis may be obtained from a subject's cells, such as from blood, urine, saliva, tissue biopsy or autopsy material. In a preferred embodiment, the DNA is obtained from blood cells obtained from a finger prick of the patient with the blood collected on absorbent paper. In a further preferred embodiment, the blood will be collected on an AmpliCard™. (University of Sheffield, Department of Medicine and Pharmacology, Royal Hallamshire Hospital, Sheffield, England S10 2JF).

The DNA may be used directly for detection or may be amplified enzymatically by using PCR or other amplification techniques prior to analysis. Oligonucleotide DNA primers that target the specific polymorphic DNA region within the genes of interest may be prepared so that in the PCR reaction amplification of the target sequences is achieved. RNA or cDNA may also be used as templates in similar fashion. The amplified DNA sequences from the template DNA may then be analyzed using restriction enzymes to determine the genetic polymorphisms present in the amplified sequences and thereby provide a genetic polymorphism profile of the patient. Restriction fragments lengths may be identified by gel analysis. Alternatively, or in conjunction, techniques such as SNP (single nucleotide polymorphisms) analysis may be employed.

Deletions and insertions can be detected by a change in size of the amplified product in comparison to the normal genotype. Point mutations can be identified by hybridizing amplified DNA to labeled Gpr100 GPCR nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers et al, Science (1985)230:1242. Sequence changes at specific locations may also be revealed by nuclease protection assays, such as RNase and S1protection or the chemical cleavage method. See Cotton et al., Proc Natl Acad Sci USA (1985) 85: 4397-4401. In another embodiment, an array of oligonucleotides probes comprising the Gpr100 GPCR nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability. (See for example: M. Chee et al., Science, Vol 274, pp 610-613 (1996)).

Single strand conformation polymorphism (SSCP) may be used to detect differences in electrophoretic mobility between mutant and wild type nucleic acids (Orita et al. (1989) Proc Natl. Acad. Sci. USA: 86:2766, see also Cotton (1993) Mutat Res 285:125-144; and Hayashi (1992) Genet Anal Tech. Appl 9:73-79). Single-stranded DNA fragments of sample and control Gpr100 nucleic acids may be denatured and allowed to renature. The secondary structure of single-stranded nucleic acids varies according to sequence, the resulting alteration in electrophoretic mobility enables the detection of even a single base change. The DNA fragments may be labelled or detected with labelled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

The diagnostic assays offer a process for diagnosing or determining a susceptibility to disorders such as Gpr100 associated diseases through detection of mutation in the Gpr100 GPCR gene by the methods described.

The presence of Gpr100 GPCR polypeptides and nucleic acids may be detected in a sample. Thus, infections and diseases as listed above can be diagnosed by methods comprising determining from a sample derived from a subject an abnormally decreased or increased level of the Gpr100 GPCR polypeptide or Gpr100 GPCR mRNA. The sample may comprise a cell or tissue sample from an organism suffering or suspected to be suffering from a disease associated with increased, reduced or otherwise abnormal Gpr100 GPCR expression, including spatial or temporal changes in level or pattern of expression. The level or pattern of expression of Gpr100 in an organism suffering from or suspected to be suffering from such a disease may be usefully compared with the level or pattern of expression in a normal organism as a means of diagnosis of disease.

In general therefore, we describe a method of detecting the presence of a nucleic acid comprising a Gpr100 GPCR nucleic acid in a sample, by contacting the sample with at least one nucleic acid probe which is specific for said nucleic acid and monitoring said sample for the presence of the nucleic acid. For example, the nucleic acid probe may specifically bind to the Gpr100 GPCR nucleic acid, or a portion of it, and binding between the two detected; the presence of the complex itself may also be detected. Furthermore, we describe a method of detecting the presence of a Gpr100 GPCR polypeptide by contacting a cell sample with an antibody capable of binding the polypeptide and monitoring said sample for the presence of the polypeptide. This may conveniently be achieved by monitoring the presence of a complex formed between the antibody and the polypeptide, or monitoring the binding between the polypeptide and the antibody. Methods of detecting binding between two entities are known in the art, and include FRET (fluorescence resonance energy transfer), surface plasmon resonance, etc.

Decreased or increased expression can be measured at the RNA level using any of the methods well known in the art for the quantitation of polynucleotides, such as, for example, PCR, RT-PCR, RNase protection, Northern blotting and other hybridization methods. Assay techniques that can be used to determine levels of a protein, such as a Gpr100 GPCR, in a sample derived from a host are well-known to those of skill in the art. Such assay methods include radioimmunoassays, competitive-binding assays, Western Blot analysis and ELISA assays.

The present document relates to a diagnostic kit for a disease or susceptibility to a disease (including an infection), for example, obesity, appetite suppression, metabolic disorders, appetite suppression. The diagnostic kit comprises a Gpr100 GPCR polynucleotide or a fragment thereof; a complementary nucleotide sequence; a Gpr100 GPCR polypeptide or a fragment thereof, or an antibody to a Gpr100 GPCR polypeptide.

### Chromosome Assays

The nucleotide sequences described here are also valuable for chromosome identification. The sequence is specifically targeted to and can hybridize with a particular location on an individual human chromosome. As described above, human Gpr100 GPCR is found to map to Homo sapiens chromosome 1q22.

The mapping of relevant sequences to chromosomes is an important first step in correlating those sequences with gene associated disease. Once a sequence has been mapped to a precise chromosomal location, the physical position of the sequence on the chromosome can be correlated with genetic map data. Such data are found, for example, in V. McKusick, Mendelian heritance in Man (available on line through Johns Hopkins University Welch Medical Library). The relationship between genes and diseases that have been mapped to the same chromosomal region are then identified through linkage analysis (coinheritance of physically adjacent genes).

The differences in the cDNA or genomic sequence between affected and unaffected individuals can also be determined. If a mutation is observed in some or all of the affected individuals but not in any normal individuals, then the mutation is likely to be the causative agent of the disease.

### Prophylactic and Therapeutic Methods

This document provides methods of treating an abnormal conditions related to both an excess of and insufficient amounts of Gpr100 GPCR activity.

If the activity of Gpr100 GPCR is in excess, several approaches are available. One approach comprises administering to a subject an inhibitor compound (antagonist) as hereinabove described along with a pharmaceutically acceptable carrier in an amount effective to inhibit activation by blocking binding of ligands to the Gpr100 GPCR, or by inhibiting a second signal, and thereby alleviating the abnormal condition.

In another approach, soluble forms of Gpr100 GPCR polypeptides still capable of binding the ligand in competition with endogenous Gpr100 GPCR may be administered. Typical embodiments of such competitors comprise fragments of the Gpr100 GPCR polypeptide.

In still another approach, expression of the gene encoding endogenous Gpr100 GPCR can be inhibited using expression blocking techniques. Known such techniques involve the use of antisense sequences, either internally generated or separately administered. See, for example, O'Connor, J Neurochem (1991) 56:560′ in Oligodeoxynucleotides as Antisense Inhibitors of Gene Expression, CRC Press, Boca Raton, Fla. (1988). Alternatively, oligonucleotides which form triple helices with the gene can be supplied. See, for example, Lee et al., Nucleic Acids Res (1979) 6:3073; Cooney et al., Science (1988) 241:456; Dervan et al., Science (1991) 251:1360. These oligomers can be administered per se or the relevant oligomers can be expressed in vivo.

For treating abnormal conditions related to an under-expression of Gpr100 GPCR and its activity, several approaches are also available. One approach comprises administering to a subject a therapeutically effective amount of a compound which activates Gpr100 GPCR, i.e., an agonist as described above, in combination with a pharmaceutically acceptable carrier, to thereby alleviate the abnormal condition. Alternatively, gene therapy may be employed to effect the endogenous production of Gpr100 GPCR by the relevant cells in the subject. For example, a Gpr100 polynucleotide may be engineered for expression in a replication defective retroviral vector, as discussed above. The retroviral expression construct may then be isolated and introduced into a packaging cell transduced with a retroviral plasmid vector containing RNA encoding a Gpr100 polypeptide such that the packaging cell now produces infectious viral particles containing the gene of interest. These producer cells may be administered to a subject for engineering cells in vivo and expression of the polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996).

### Formulation and Administration

Peptides, such as the soluble form of Gpr100 GPCR polypeptides, and agonists and antagonist peptides or small molecules, may be formulated in combination with a suitable pharmaceutical carrier. Such formulations comprise a therapeutically effective amount of the polypeptide or compound, and a pharmaceutically acceptable carrier or excipient. Such carriers include but are not limited to, saline, buffered saline, dextrose, water, glycerol, ethanol, and combinations thereof. Formulation should suit the mode of administration, and is well within the skill of the art. We further describe pharmaceutical packs and kits comprising one or more containers filled with one or more of the ingredients of the aforementioned compositions.

Polypeptides and other compounds may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible. Administration of these compounds may also be topical and/or localize, in the form of salves, pastes, gels and the like.

The dosage range required depends on the choice of peptide, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

### Pharmaceutical Compositions

The present document also provides a pharmaceutical composition comprising administering a therapeutically effective amount of the Gpr100 polypeptide, polynucleotide, peptide, vector or antibody and optionally a pharmaceutically acceptable carrier, diluent or excipients (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

### Vaccines

Another embodiment relates to a method for inducing an immunological response in a mammal which comprises inoculating the mammal with the Gpr100 GPCR polypeptide, or a fragment thereof, adequate to produce antibody and/or T cell immune response to protect said animal from obesity, appetite suppression, metabolic disorders, among others.

Yet another embodiment relates to a method of inducing immunological response in a mammal which comprises delivering a Gpr100 GPCR polypeptide via a vector directing expression of a Gpr100 GPCR polynucleotide in vivo in order to induce such an immunological response to produce antibody to protect said animal from diseases.

A further embodiment relates to an immunological/vaccine formulation (composition) which, when introduced into a mammalian host, induces an immunological response in that mammal to a Gpr100 GPCR polypeptide wherein the composition comprises a Gpr100 GPCR polypeptide or Gpr100 GPCR gene. The vaccine formulation may further comprise a suitable carrier.

Since the Gpr100 GPCR polypeptide may be broken down in the stomach, it is preferably administered parenterally (including subcutaneous, intramuscular, intravenous, intradermal etc. injection). Formulations suitable for parenteral administration include aqueous and non-aqueous sterile injection solutions which may contain anti-oxidants, buffers, bacteriostats and solutes which render the formulation instonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Vaccines may be prepared from one or more Gpr100 polypeptides or peptides.

The preparation of vaccines which contain an immunogenic polypeptide(s) or peptide(s) as active ingredient(s), is known to one skilled in the art. Typically, such vaccines are prepared as injectables, either as liquid solutions or suspensions; solid forms suitable for solution in, or suspension in, liquid prior to injection may also be prepared. The preparation may also be emulsified, or the protein encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof.

In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

Further examples of adjuvants and other agents include aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, Corynebacterium parvum (Propionobacterium acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.).

Typically, adjuvants such as Amphigen (oil-in-water), Alhydrogel (aluminum hydroxide), or a mixture of Amphigen and Alhydrogel are used. Only aluminum hydroxide is approved for human use.

The proportion of immunogen and adjuvant can be varied over a broad range so long as both are present in effective amounts. For example, aluminum hydroxide can be present in an amount of about 0.5% of the vaccine mixture (Al₂O₃ basis). Conveniently, the vaccines are formulated to contain a final concentration of immunogen in the range of from 0.2 to 200 μg/ml, preferably 5 to 50 μg/ml, most preferably 15 μg/ml.

After formulation, the vaccine may be incorporated into a sterile container which is then sealed and stored at a low temperature, for example 4° C., or it may be freeze-dried. Lyophilisation permits long-term storage in a stabilised form.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

The Gpr100 polypeptides may be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

### Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The pharmaceutical and vaccine compositions may be administered by direct injection. The composition may be formulated for parenteral, mucosal, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestible solution; a parenteral route where delivery is by an ‘injectable form, such as, for example,’ an intravenous, intramuscular or subcutaneous route.

The term “co-administered” means that the site and time of administration of each of for example, the Gpr100 polypeptide and an additional entity such as adjuvant are such that the necessary modulation of the immune system is achieved. Thus, whilst the polypeptide and the adjuvant may be administered at the same moment in time and at the same site, there may be advantages in administering the polypeptide at a different time and to a different site from the adjuvant. The polypeptide and adjuvant may even be delivered in the same delivery vehicle—and the polypeptide and the antigen may be coupled and/or uncoupled and/or genetically coupled and/or uncoupled.

The polypeptide, polynucleotide, peptide, nucleotide, antibody as described and optionally an adjuvant may be administered separately or co-administered to the host subject as a single dose or in multiple doses.

The vaccine composition and pharmaceutical compositions may be administered by a number of different routes such as injection (which includes parenteral, subcutaneous and intramuscular injection) intranasal, mucosal, oral, intra-vaginal, urethral or ocular administration.

The vaccines and pharmaceutical compositions may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, may be 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer.

Alternatively, a therapeutic compounds or agents identified by the methods described herein may be used for the treatment or prevention of a diabetes related disorder or a weight related disorder. In one aspect, the compound or agent may be a natural, synthetic, semi-synthetic, or recombinant Gpr100 receptor gene, Gpr100 receptor gene product, or fragment thereof as well as an analog of the gene, gene product or fragment. In another aspect, the compound may be an antibody specific for the gene or gene product, antisense DNA or RNA, or an organic or inorganic small molecule. In a preferred embodiment, the compound or agent will have an affect on the activity, expression or function of the Gpr100 receptor gene or Gpr100 receptor gene product.

Methods for the treatment of a diabetes related disorder or a weight related disorder are provided. In one aspect, a therapeutically effective amount of an agent that is capable of modulating Gpr100 receptor is administered to a subject in need thereof. The agent capable of modulating Gpr100 receptor includes but is not limited to an antibody specific for the gene or gene product, antisense DNA or RNA, or an organic or inorganic small molecule. The Gpr100 receptor modulator may be administered alone, or as part of a pharmaceutically acceptable composition. For example, the Gpr100 receptor modulator may be administered in combination with other Gpr100 receptor agonists or antagonists, or with other pharmaceutically active compounds. For example, the additional pharmaceutically active compounds may include anti-diabetic agents or anti-obesity agents that are known in the art, or agents meant for the treatment of other symptoms or diseases.

Methods for the treatment of a diabetes related disorder or a weight related disorder comprise administering a therapeutically effective amount of Gpr100 receptor gene or Gpr100 receptor to a subject in need thereof.

### Further Aspects

Further aspects and embodiments of the invention are now set out in the following numbered Paragraphs; it is to be understood that the invention encompasses these aspects:

Paragraph 1. A Gpr100 GPCR polypeptide comprising the amino acid sequence shown in SEQ ID NO: 3 of SEQ ID NO: 5, or a homologue, variant or derivative thereof.

Paragraph 2. A nucleic acid encoding a polypeptide according to Paragraph 1.

Paragraph 3. A nucleic acid according to Paragraph 2, comprising the nucleic acid sequence shown in SEQ ID NO: 1, SEQ ID NO: 2 or SEQ ID NO: 4, or a homologue, variant or derivative thereof.

Paragraph 4. A polypeptide comprising a fragment of a polypeptide according to Paragraph 1.

Paragraph 5. A polypeptide according to Paragraph 3 which comprises one or more regions which are homologous between SEQ ID NO: 3 and SEQ ID NO: 5, or which comprises one or more regions which are heterologous between SEQ ID NO: 3 and SEQ ID NO: 5.

Paragraph 6. A nucleic acid encoding a polypeptide according to Paragraph 4 or 5.

Paragraph 7. A vector comprising a nucleic acid according to Paragraph 2, 3, or 6.

Paragraph 8. A host cell comprising a nucleic acid according to Paragraph 2, 3, or 6, or vector according to Paragraph 7.

Paragraph 9. A transgenic non-human animal comprising a nucleic acid according to Paragraph 2, 3 or 6, or a vector according to Paragraph 7.

Paragraph 10. A transgenic non-human animal according to Paragraph 9 which is a mouse.

Paragraph 11. Use of a polypeptide according to Paragraph 1, 4 or 5 in a method of identifying a compound which is capable of interacting specifically with a G protein coupled receptor.

Paragraph 12. Use of a transgenic non-human animal according to Paragraph 9 or 10 in a method of identifying a compound which is capable of interacting specifically with a G protein coupled receptor.

Paragraph 13. A method for identifying an antagonist of a Gpr100 GPCR, the method comprising contacting a cell which expresses Gpr100 receptor with a candidate compound and determining whether the level of cyclic AMP (cAMP) in the cell is lowered as a result of said contacting.

Paragraph 14. A method for identifying a compound capable of lowering the endogenous level of cyclic AMP in a cell which method comprises contacting a cell which expresses a Gpr100 GPCR with a candidate compound and determining whether the level of cyclic AMP (cAMP) in the cell is lowered as a result of said contacting.

Paragraph 15. A method of identifying a compound capable of binding to a Gpr100 GPCR polypeptide, the method comprising contacting a Gpr100 GPCR polypeptide with a candidate compound and determining whether the candidate compound binds to the Gpr100 GPCR polypeptide.

Paragraph 16. A compound identified by a method according to any of Paragraph s 11 to 15.

Paragraph 17. A compound capable of binding specifically to a polypeptide according to Paragraph 1, 4 or 5.

Paragraph 18. Use of a polypeptide according to Paragraph 1, 4 or 5, or part thereof or a nucleic acid according to Paragraph 2, 3 or 6, in a method for producing antibodies.

Paragraph 19. An antibody capable of binding specifically to a polypeptide according to Paragraph 1, 4 or 5, or part thereof or a polypeptide encoded by a nucleotide according to Paragraph 2, 3 or 6, or part thereof.

Paragraph 20. A pharmaceutical composition comprising any one or more of the following: a polypeptide according to Paragraph 1, 4 or 5, or part thereof; a nucleic acid according to Paragraph 2, 3 or 6, or part thereof, a vector according to Paragraph 7; a cell according to Paragraph 8; a compound according to Paragraph 16 or 17; and an antibody according to Paragraph 19, together with a pharmaceutically acceptable carrier or diluent.

Paragraph 21. A vaccine composition comprising any one or more of the following: a polypeptide according to Paragraph 1, 4 or 5, or part thereof; a nucleic acid according to Paragraph 2, 3 or 6, or part thereof, a vector according to Paragraph 7; a cell according to Paragraph 8; a compound according to Paragraph 16 or 17; and an antibody according to Paragraph 19.

Paragraph 22. A diagnostic kit for a disease or susceptibility to a disease comprising any one or more of the following: a polypeptide according to Paragraph 1, 4 or 5, or part thereof, a nucleic acid according to Paragraph 2, 3 or 6, or part thereof, a vector according to Paragraph 7; a cell according to Paragraph 8; a compound according to Paragraph 16 or 17; and an antibody according to Paragraph 19.

Paragraph 23. A method of treating a patient suffering from a disease associated with enhanced activity of a Gpr100 GPCR, which method comprises administering to the patient an antagonist of Gpr100 GPCR.

Paragraph 24. A method of treating a patient suffering from a disease associated with reduced activity of a Gpr100 GPCR, which method comprises administering to the patient an agonist of Gpr100 GPCR.

Paragraph 25. A method according to Paragraph 23 or 24, in which the Gpr100 GPCR comprises a polypeptide having the sequence shown in SEQ ID NO: 3 or SEQ ID NO: 5.

Paragraph 26. A method for treating and/or preventing a disease in a patient, which comprises the step of administering any one or more of the following to the patient: a polypeptide according to Paragraph 1, 4 or 5, or part thereof; a nucleic acid according to Paragraph 2, 3 or 6, or part thereof; a vector according to Paragraph 7; a cell according to Paragraph 8; a compound according to Paragraph 16 or 17; an antibody according to Paragraph 19; a pharmaceutical composition according to Paragraph 20; and a vaccine according to Paragraph 20.

Paragraph 27. An agent comprising a polypeptide according to Paragraph 1, 4 or 5, or part thereof; a nucleic acid according to Paragraph 2, 3 or 6, or part thereof, a vector according to Paragraph 7; a cell according to Paragraph 8; a compound according to Paragraph 16 or 17; and/or an antibody according to Paragraph 19, said agent for use in a method of treatment or prophylaxis of disease.

Paragraph 28. Use of a polypeptide according to Paragraph 1, 4 or 5, or part thereof; a nucleic acid according to Paragraph 2, 3 or 6, or part thereof; a vector according to Paragraph 7; a cell according to Paragraph 8; a compound according to Paragraph 16 or 17; and an antibody according to Paragraph 19, for the preparation of a pharmaceutical composition for the treatment or prophylaxis of a disease.

Paragraph 29. A non-human transgenic animal, characterised in that the transgenic animal comprises an altered Gpr100 gene.

Paragraph 30. A non-human transgenic animal according to Paragraph 29, in which the alteration is selected from the group consisting of: a deletion of Gpr100, a mutation in Gpr100 resulting in loss of function, introduction of an exogenous gene having a nucleotide sequence with targeted or random mutations into Gpr100, introduction of an exogenous gene from another species into Gpr100, and a combination of any of these.

Paragraph 31. A non-human transgenic animal having a functionally disrupted endogenous Gpr100 gene, in which the transgenic animal comprises in its genome and expresses a transgene encoding a heterologous Gpr100 protein.

Paragraph 32. A nucleic acid construct for functionally disrupting a Gpr100 gene in a host cell, the nucleic acid construct comprising: (a) a non-homologous replacement portion; (b) a first homology region located upstream of the non-homologous replacement portion, the first homology region having a nucleotide sequence with substantial identity to a first Gpr100 gene sequence; and (c) a second homology region located downstream of the non-homologous replacement portion, the second homology region having a nucleotide sequence with substantial identity to a second Gpr100 gene sequence, the second Gpr100 gene sequence having a location downstream of the first Gpr100 gene sequence in a naturally occurring endogenous Gpr100 gene.

Paragraph 33. A process for producing a Gpr100 GPCR polypeptide, the method comprising culturing a host cell according to Paragraph 8 under conditions in which a nucleic acid encoding a Gpr100 GPCR polypeptide is expressed.

Paragraph 34. A method of detecting the presence of a nucleic acid according to Paragraph 2, 3 or 6 in a sample, the method comprising contacting the sample with at least one nucleic acid probe which is specific for said nucleic acid and monitoring said sample for the presence of the nucleic acid.

Paragraph 35. A method of detecting the presence of a polypeptide according to Paragraph 1, 4 or 5 in a sample, the method comprising contacting the sample with an antibody according to Paragraph 19 and monitoring said sample for the presence of the polypeptide.

Paragraph 36. A method of diagnosis of a disease or syndrome caused by or associated with increased, decreased or otherwise abnormal expression of Gpr100 GPCR, the method comprising the steps of: (a) detecting the level or pattern of expression of Gpr100 GPCR in an animal suffering or suspected to be suffering from obesity including prevention of obesity or weight gain, appetite suppression, lipid metabolism disorders including hyperlipidemia, dyslipoidemia, and hypertriglyceridemia, diabetes and related disorders include but are not limited to: Type II Diabetes, impaired glucose tolerance, insulin resistance syndromes, syndrome X, hyperglycemia, acute pancreatitis, cardiovascular diseases, hypertension, cardiac hypertrophy, and hypercholesterolemia; and (b) comparing the level or pattern of expression with that of a normal animal.

Paragraph A1. A method of identifying a molecule suitable for the treatment, prophylaxis or alleviation of a Gpr100 associated disease, in particular diabetes and obesity, the method comprising determining whether a candidate molecule is an agonist or antagonist of Gpr100 polypeptide, in which the Gpr100 polypeptide comprises the amino acid sequence shown in SEQ ID NO: 3 or SEQ ID NO: 5, or a sequence which is at least 90% identical thereto.

Paragraph A2. A method according to Paragraph A1, in which the Gpr100 polypeptide is encoded by a nucleic acid sequence shown in SEQ ID NO: 1, SEQ ID NO: 2 or SEQ ID NO: 4, or a sequence which is at least 90% identical thereto.

Paragraph A3. A method according to Paragraph A1 or A2, comprising exposing the candidate molecule to a Gpr100 polypeptide, and detecting a change in intracellular calcium level as a result of such exposure.

Paragraph A4. A method according to Paragraph A1 or A2, comprising exposing a non-human animal or a portion thereof, preferably a cell, tissue or organ, to a candidate molecule and determining whether a biological parameter of the animal is changed as a result of the contacting.

Paragraph A5. A method according to Paragraph A4, in which the biological parameter is selected from the group consisting of serum glucose levels, body weight, glucagon levels, fat percentage.

Paragraph A6. Use of a transgenic non-human animal having a functionally disrupted endogenous Gpr100, or an isolated cell or tissue thereof, as a model for glucose regulation or a Gpr100 associated disease, preferably obesity or diabetes.

Paragraph A7. A use according to Paragraph A6, in which the transgenic non-human animal comprises a functionally disrupted Gpr100 gene, preferably comprising a deletion in a Gpr100 gene or a portion thereof.

Paragraph A8. A use or method according to Paragraph A6 or A7, in which the transgenic non-human animal displays a change in any one or more of the following phenotypes when compared with a wild type animal: decreased serum glucose levels, increased body weight, higher fat percentage.

Paragraph A9. A use or method according to Paragraph A6, A7 or A8, in which the transgenic non-human animal is a rodent, preferably a mouse.

Paragraph A10. Use of a Gpr100 polypeptide comprising an amino acid sequence shown in SEQ ID NO: 3 or SEQ ID NO: 5, or a sequence which is at least 90% identical thereto, for the identification of an agonist or antagonist thereof for the treatment, prophylaxis of a Gpr100 associated disease, preferably obesity or diabetes.

Paragraph A11. Use of a Gpr100 polynucleotide comprising a nucleic acid sequence shown in SEQ ID NO: 1, SEQ ID NO: 2 or SEQ ID NO: 4, or a sequence which is at least 90% identical thereto, for the identification of an agonist or antagonist thereof for the treatment, prophylaxis of a Gpr100 associated disease, preferably obesity or diabetes.

Paragraph A12. Use of a non-human animal or a portion thereof, preferably a cell, tissue or organ, in a method of identifying an agonist or antagonist of Gpr100 polypeptide for use in the treatment, prophylaxis or alleviation of a Gpr100 associated disease, preferably diabetes or obesity.

Paragraph A13. Use of a an agonist or antagonist identified by a method or use according to any preceding Paragraph A for the treatment, prophylaxis or alleviation of a Gpr100 associated disease, preferably obesity or diabetes.

Paragraph A14. A method of modulating the regulation of glucose, fat metabolism or weight gain in an individual by modulating the activity of a Gpr100 polypeptide in the individual comprising an amino acid sequence shown in SEQ ID NO: 3 or SEQ ID NO: 5, or a sequence which is at least 90% identical thereto.

Paragraph A15 A method according to Paragraph A14, comprising administering an agonist or antagonist of Gpr100 to the individual.

Paragraph A16. A method of treating an individual suffering from a Gpr100 associated disease, the method comprising increasing or decreasing the activity or amount of Gpr100 polypeptide in the individual.

Paragraph A17. A method according to Paragraph A16, which method comprises administering a Gpr100 polypeptide, an agonist of Gpr100 polypeptide or an antagonist of Gpr100 to the individual

Paragraph A18. A method of diagnosis of a Gpr100 associated disease, the method comprising the steps of (a) detecting the level or pattern of expression of Gpr100 polypeptide in an animal suffering or suspected to be suffering from such a disease; and (b) comparing the level or pattern of expression with that of a normal animal.

Paragraph A19. A method of diagnosis of a Gpr100 associated disease, the method comprising detecting a change in a biological parameter as set out in Paragraph A5 in an individual suspected of suffering from that disease.

Paragraph A20. A diagnostic kit for susceptibility to a Gpr100 associated disease, preferably obesity or diabetes, comprising any one or more of the following: a Gpr100 polypeptide or part thereof, an antibody against a Gpr100 polypeptide; or a nucleic acid capable of encoding such.

Paragraph A21. A method according to any preceding Paragraph A, in which the a Gpr100 associated disease is selected from the group consisting of obesity including prevention of obesity or weight gain, appetite suppression, metabolic disorders, diabetes, including Type I diabetes and Type II diseases, and related disorders and weight related disorders, impaired glucose tolerance, insulin resistance syndromes, syndrome X, peripheral neuropathy, diabetic neuropathy, diabetes associated proteinuria, lipid metabolism disorders including hyperglycemia, hyperlipidemia, dyslipidemia, hypertriglyceridemia, acute pancreatitis, cardiovascular diseases, peripheral vascular disease, hypertension, cardiac hypertrophy, ischaemic heart disease, hypercholesterolemia, obesity, and prevention of obesity or weight gain.

### EXAMPLES

### Example 1

### Transgenic Gpr100 Knock Out Mouse

Construction of Gpr100 Gene Targeting Vector

The Gpr100 gene was identified bio-informatically using homology searches of genome databases. A 62 kb genomic contig was assembled from various databases. This contig provided sufficient flanking sequence information to enable the design of homologous arms to clone into the targeting vector.

The murine Gpr100 gene has 1 coding exon. The targeting strategy is designed to remove a large portion of the coding sequence including the majority transmembrane domains. A 3.1 kb 5′ homologous arm and a 1.8 kb 3′ homologous arm flanking the region to be deleted are amplified by PCR and the fragments are cloned into the targeting vector. The 5′ end of each oligonucleotide primer used to amplify the arms is synthesised to contain a different recognition site for a rare-cutting restriction enzyme, compatible with the cloning sites of the vector polylinkers and absent from the arms themselves. In the case of Gpr100, the primers are designed as listed in the primer table below, with 5′ arm cloning sites of NotI/SpeI and 3′arm cloning sites of AscI/FseI (the structure of the targeting vector used, including the relevant restriction sites, is shown in FIG. 2).

In addition to the arm primer pairs (5′ armF/5′ armR) and (3′ armF/3′armR), further primers specific to the Gpr100 locus are designed for the following purposes: 5′ and 3′ probe primer pairs (5′prF/5′ prR and 3′prF/3′ prR) to amplify two short 150-300 bp fragments of non-repetitive genomic DNA external to and extending beyond each arm, to allow Southern analysis of the targeted locus, in isolated putative targeted clones; a mouse genotyping primer pair (hetF and hetR) which allows differentiation between wild-type, heterozygote and homozygous mice, when used in a multiplex PCR with a vector specific primer, in this case, Asc350; and lastly, a target screening primer (3′ scr) which anneals downstream of the end of the 3′ arm region, and which produces a target event specific 1.9 kb amplimer when paired with a primer specific to the 3′ end of the vector (TK5IBLMNL), in this case Asc53. This amplimer can only be derived from template DNA from cells where the desired genomic alteration has occurred and allows the identification of correctly targeted cells from the background of clones containing randomly integrated copies of the vector. The location of these primers and the genomic structure of the regions of the Gpr100 locus used in the targeting strategy is shown in SEQ ID NO: 19.

TABLE 1
Gpr100 Primer Sequences

| | | |
|---|---|---|
| musGpr100C 5′prF | TTGTGCAGAGTTCAATGGAGAATGTTG- | SEQ ID NO: 6 |
| musGpr100C 5′prR | CCAGAAACACTCTACGCCTGTCACCTG- | SEQ ID NO: 7 |
| musGpr100C 5′armF Not | TttgcggccgcAAAGTGACTCATGCTGCTCCCATCTTC- | SEQ ID NO: 8 |
| musGpr100C 5′armR Spe | AaaactagTCCCAGCAAGCCAATGATACCTACAAG- | SEQ ID NO: 9 |
| musGpr100C 3′armF Asc | TttggcgcgCCTGGGACAGTACTTTCTACACCTTTC- | SEQ ID NO: 10 |
| musGpr100C 3′armR Fse | TttggccggccTCCATTTAAGAAGAGATCTTGAGCCAG- | SEQ ID NO: 11 |
| musGpr100C 3′scr | TGGATCCTTTTATTTTGGAGACTGAAC- | SEQ ID NO: 12 |
| musGpr100C 3′prF | CCTGGCTCAAGATCTCTTCTTAAATGG- | SEQ ID NO: 13 |
| musGpr100C 3′prR | GGTGAGCAATCAGATCATGAGACTTAC- | SEQ ID NO: 14 |
| musGpr100C hetF | GCTTACCAGCTACAGAGGGTAGTTCTG- | SEQ ID NO: 15 |

| | | |
|---|---|---|
| musGpr 100 hetR3 | TGATGGGAAGGATGTAAGTATGAAAGGTG- | SEQ.ID NO: 16 |
| Asc350 | GTCGTGACCCATGGCGATGCCTGCTTG- | SEQ ID NO: 17 |
| Asc53 | CGGATCCACTAGATAACTTCGTATAGC- | SEQ ID NO: 18 |

The position of the homology arms is chosen to functionally disrupt the Gpr100 gene. A targeting vector is prepared where the Gpr100 region to be deleted is replaced with non-homologous sequences composed of an endogenous gene expression reporter (a frame independent lacZ gene) upstream of a selection cassette composed of a promoted neomycin phosphotransferase (neo) gene arranged in the same orientation as the Gpr100 gene.

Once the 5′ and 3′ homology arms have been cloned into the targeting vector TK5IBLMNL, a large highly pure DNA preparation is made using standard molecular biology techniques. 20 μg of the freshly prepared endotoxin-free DNA is restricted with another rare-cutting restriction enzyme PmeI, present at a unique site in the vector backbone between the ampicillin resistance gene and the bacterial origin of replication. The linearized DNA is then precipitated and resuspended in 100 μl of Phosphate Buffered Saline, ready for electroporation.

24 hours following electroporation the transfected cells are cultured for 9 days in medium containing 200 μg/ml neomycin. Clones are picked into 96 well plates, replicated and expanded before being screened by PCR (using primers 3′ scr and Asc53, as described above) to identify clones in which homologous recombination has occurred between the endogenous Gpr100 gene and the targeting construct. Positive clones can be identified at a rate of 1 to 5%. These clones are expanded to allow replicas to be frozen and sufficient high quality DNA to be prepared for Southern blot confirmation of the targeting event using the external 5′ and 3′ probes prepared as described above, all using standard procedures (Russ et al, Nature 2000 Mar. 2; 404(6773):95-99). When Southern blots of DNA digested with diagnostic restriction enzymes are hybridized with an external probe, homologously targeted ES cell clones are verified by the presence of a mutant band as well an unaltered wild-type band. For instance, using the 5′ probe, SpeI digested genomic DNA will give a 15.7 kb wild-type band and a 8.5 kb targeted band; and with the 3′ probe, SpeI cut DNA will give a 15.7 kb wild-type band and an 11.5 kb targeted band.

Generation of Gpr100 GPCR Deficient Mice

C57BL/6 female and Male mice are mated and blastocysts are isolated at 3.5 days of gestation. 10-12 cells from a chosen clone are injected per blastocyst and 7-8 blastocysts are implanted in the uterus of a pseudopregnant F1 female. A litter of chimeric pups are born containing several high level (up to 100%) agouti males (the agouti coat colour indicates the contribution of cells descended from the targeted clone). These male chimeras are mated with female MF1 and 129 mice, and germline transmission is determined by the agouti coat colour and by PCR genotyping respectively.

PCR Genotyping is carried out on lysed tail clips, using the primers hetF and hetR with a third, vector specific primer (Asc350). This multiplex PCR allows amplification from the wild-type locus (if present) from primers hetF and hetR giving a 285 bp band. The site for hetF is deleted in the knockout mice, so this amplification will fail from a targeted allele. However, the Asc350 primer will amplify a 397 by band from the targeted locus, in combination with the hetR primer which anneals to a region just inside the 3′ arm. Therefore, this multiplex PCR reveals the genotype of the litters as follows: wild-type samples exhibit a single 285 by band; heterozygous DNA samples yield two bands at 285 by and 397 bp; and the homozygous samples will show only the target specific 397 by band.

Transgenic mice having a disruption in the Gpr100 receptor gene exhibit a metabolic abnormality. Specifically after exposure to a high fat diet, the transgenic mice gain more body weight and body fat relative to wild-type control mice suggesting that the Gpr100 receptor is involved in the regulation of fat and glucose metabolism. This weight gain may provide a valuable insight into treatment and/or prevention of related disorders such as diabetes and obesity. As such, Gpr100 receptor may be useful as a target for the discovery of therapeutic agents for the treatment of diabetes related disorders.

### Example 2

### Biological Data: Serum Chemistry: Blood

Samples were collected via a terminal cardiac puncture in a syringe. One hundred microliters of each whole blood sample was transferred into a tube pre-filled with EDTA. The remainder of the blood sample was converted to serum by centrifugation in a serum tube with a gel separator. Each serum sample was then analyzed as described below. Non-terminal blood samples for aged mice are collected via retro-orbital venous puncture in capillary tubes. This procedure yields approximately 200 uL of whole blood that is either transferred into a serum tube with a gel separator for serum chemistry analysis (see below), or into a tube pre-filled with EDTA for haematology analysis.

The serum was analyzed using standard laboratory techniques and assays for the following parameters: insulin, alanine aminotransferase, albumin, alkaline phosphatase, aspartate transferase, bicarbonate, total bilirubin, blood urea nitrogen, calcium, chloride, cholesterol, creatine kinase, creatinine, globulin, glucose, high density lipoproteins (HDL), lactate dehydrogenase, low density lipoproteins (LDL), osmolality, phosphorus, potassium, total protein, sodium, and triglycerides.

### Example 3

### Biological Data: Histological and Densitometric Analysis

Adipose Tissue Histology After Fasting

Mice (n=4 for both mutants and wildtypes) were fasted for 16 h, killed then the white adipose tissue was dissected, fixed in 4% paraformaldehyde, embedded in wax, sectioned and stained using Hematoxylin and Eosin according to standard histology protocols.

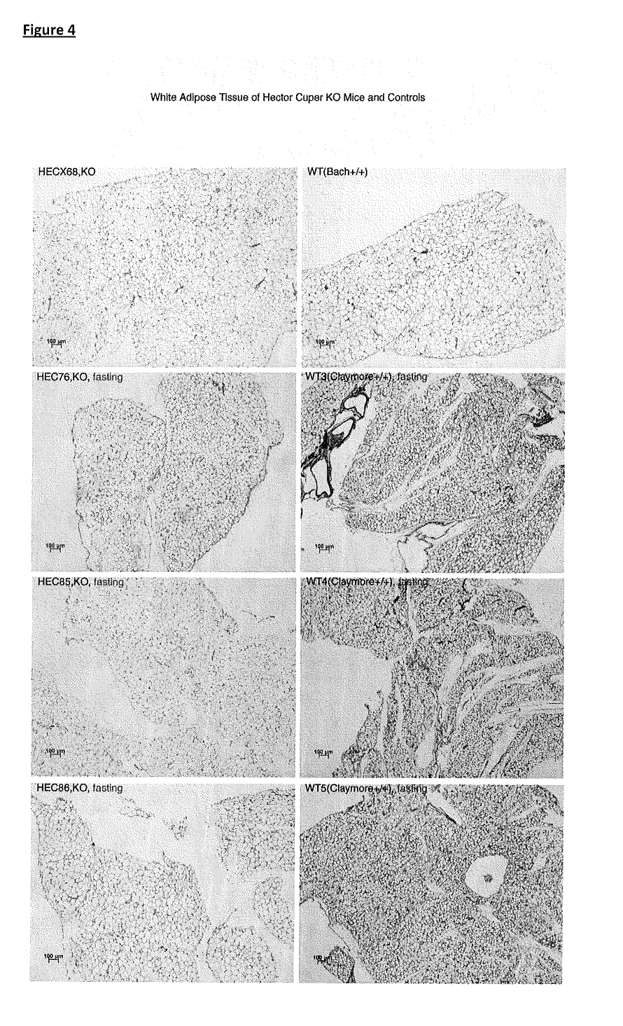

The results are shown in FIG. 4. FIG. 4 shows white adipose tissue from 4 mutants and 4 wildtypes, the top panel shows control non fasted animals, and the bottom 3 panels shows tissue from fasted animals.

The data shows that after fasting, wild type mice have a reduction in, adipocyte cell size. This reduction is not observed in KO mice and therefore indicate that KO are unable to mobilize fat during fasting.

Densitometry

Mice were killed and analyzed using a Piximus™ densitometer. An x-ray source exposed the mice to a beam of both high and low energy x-rays. The ratio of attenuation of the high and low energies allowed the separation of bone from soft tissue, and, from within the tissue samples, lean and fat. Densitometric data including Bone Mineral Density (BMD presented as g/cm2), Bone Mineral Content (BMC in g), bone and tissue area, total tissue mass, and fat as a percent of body soft tissue (presented as fat %) were obtained and recorded.

When compared to age- and gender-matched control mice, homozygous mutant mice exhibited increased fat as a percentage of body soft tissue (fat %). This increased fat percentage was observed in female homozygous mutant mice at approximately 49 days of age. This increase in fat percentage was further seen when mice were exposed to a normal diet (not a high fat diet).

Metrics: Body lengths and body weights were recorded throughout the high fat diet challenge.

Results: Knockout mice exhibited metabolic characteristics of diabetes and obesity.

Knockout mice were subjected to a high fat diet challenge for about 8 weeks, and subjected to a Glucose Tolerance Test. Densitometric measurements and body weights and lengths (metrics) were also recorded post-high fat diet challenge.

Glucose Tolerance Test (GTT): Mice were fasted for about 5 hours and tail vein blood glucose levels were measured before injection by collecting about 5 to 10 microliters of blood from the tail tip and using glucometers (Glucometer Elite, Bayer Corporation, Mishawaka, Ind.). The glucose values were used for time T=0. Mice were weighed at t=0 and glucose was administered orally or by intra-peritoneal injection at a dose of about 2 grams per kilogram of body weight. Plasma glucose concentrations were measured at about 15,30, 60,90, and 120 minutes after injection by the same method used to measure basal (T=0) blood glucose.

Mice were returned to cages with access to food ad libitum for about one week, after which the GTT is repeated. Glucose values for both tests were averaged for statistical analysis. Pair-wise statistical significance was established using a Student t-test. Statistical significance is defined as P<0.05.

### Example 4

### Biological Data: Insulin Suppression Test (IST)

Tail vein glucose levels and body weight are measured at t=0 as in the GTT above. Insulin (Humulin R, Eli Lilly and Company, Indianapolis, Ind.) is administered by intraperitoneal injection at about 0.5 or 0.7 Units per kilogram body weight for male mice on chow diet (or on the high fat diet). In a few cases when female mice are used, 0.5 Units of insulin per kilogram body weight is used. Plasma glucose levels are measured at about 15,30, 60,90, and 120 minutes after insulin injection and presented as the percent of basal glucose. The resulting glucose levels may represent the sensitivity of the mouse to insulin, such as, for example, the ability of certain tissues to uptake glucose in response to insulin.

### Example 5

### Biological Data:Glucose-Stimulated Insulin Secretion Test (GSIST)

TAIL vein blood samples are taken before the test to measure serum insulin levels at T=0. Glucose is administered orally or by intraperitoneal injection at approximately 2 grams per kilogram mouse body weight. Tail vein blood samples are then collected at about 7.5, 15,30, and 60 minutes after the glucose loading. Serum insulin levels are determined by an ELISA kit (Crystal Chem Inc., Chicago, Ill.).

Metabolic Chamber

Mice are individually housed in a metabolic chamber (Columbus Instruments, Columbus, Ohio). Metabolic rates (VO2/Kg/hr), respiratory exchange ratio (RER=VC02/V02), ambulatory/locomotor activities and food and water intakes are monitored for a period of about 48 hours. Data are recorded about every 48 minutes. Mice are then fasted overnight for about 18 hours and the same data are collected for approximately the next 24 hours in order to observe the hyperphagic responses of the mice to overnight fasting.

Densitometry

Body fat composition and bone mineral density (BMD) are analyzed by a DEXA (dual energy X-ray absorptiometry) densitometer (Piximus, GE Medical Systems Lunar, Madison, Wis.).

Necropsy

Blood is collected by cardiac puncture for standard serum chemistry and for measurement of serum levels of leptin by ELISA. Mesenteric, epididymal, inguinal and brown fat pads are individually weighed to assess fat distribution. Pancreas, liver and kidney are collected for histological analysis.

A role for the Gpr100 receptor gene in diabetes and glucose tolerance would be supported should the Gpr100 receptor gene deficient behave differently in the above tests when compared to wild-type mice.

### Example 6

### Biological Data: Long Term High Fat and Low Fat Diet Study, Body Weight and Fat Content

The homozygous animals have no obvious phenotype in the general survey (modified Irwin), a battery of behavioural and neurological tests.

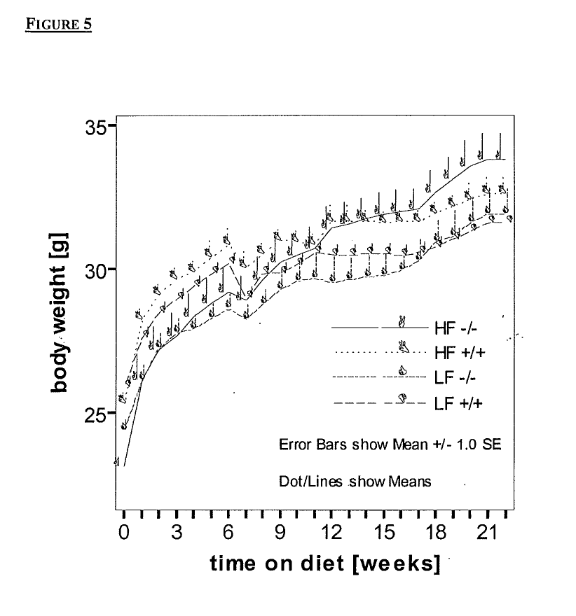

To investigate their energy homeostasis in more detail the animals are subjected to a 24 week high fat diet (high fat diet, RFD: 35 kcal % carbohydrates, 45 kcal % fat) or an iso-caloric control diet (low fat diet, LFD: 70 kcal % carbohydrates, 10 kcal % fat), respectively. The body weight is not significantly different between the cohorts for the whole study period. Nevertheless, in the initial period wild type animals seem to gain weight at a faster rate then the knockout animals (FIG. 5).

Consistent with this a separate analysis of the first 6 weeks reveals a significant effect of the genotype (p=0.009, general linear model for repeated measures (GLM)). For the last 8 weeks of the study a trend is detected for diet effects (p=0.058, GLM) suggesting a higher body weight for the high fat diet cohorts of wild type and knockout animals.

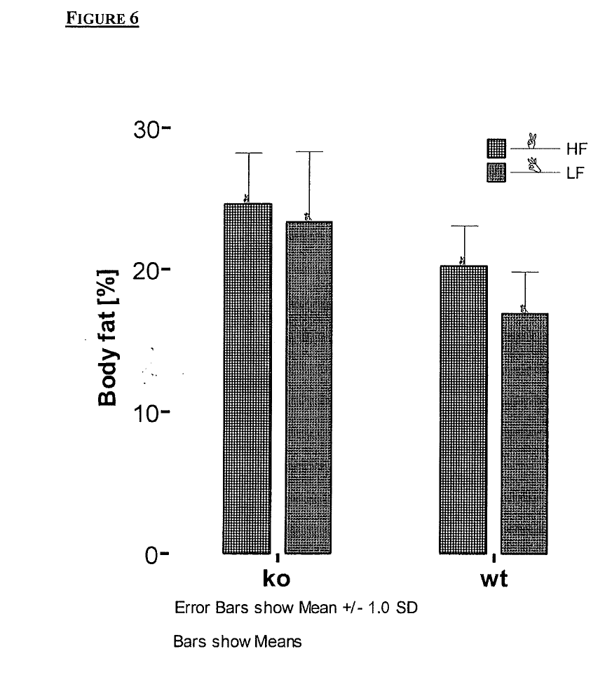

Body composition is measured in fed animals at week 23 of the study by DEXA analysis. The high fat diet causes a more pronounced increase in body fat content than the iso-caloric control diet in the wild type animals (FIG. 6). In knockout animals no diet effect is observed. Instead, the body fat content is elevated in all knockout animals irrespective of the diet when compared to wild type animals on either diet (p<0.0001 for the effect of genotype, GLM).

### Example 7

### Biological Data: Long Term High Fat and Low Fat Diet Study: Glucose Tolerance Test

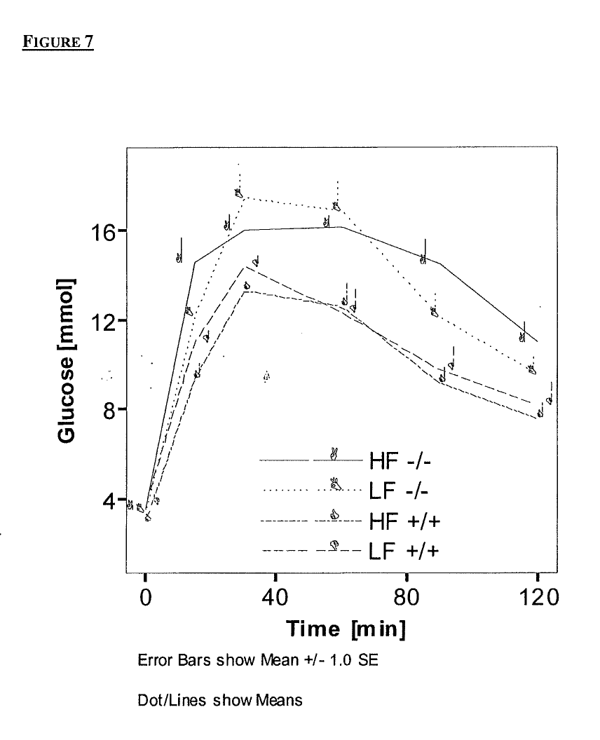

Next we were interested to explore whether this elevation in body fat content is accompanied by an insulin resistance. As expected, a significantly impaired glucose tolerance test (GTT) is detected in knockout animals again without any diet effects (FIG. 7, p=0.0006 for genotype effect, GLM).

### Example 8

### Biological Data: Measurement of Glucagon Levels Over Time During Fasting

Glucagon levels are measured, following manufactures instructions, in the terminal sample described above using a Glucagon RIA (Linco).

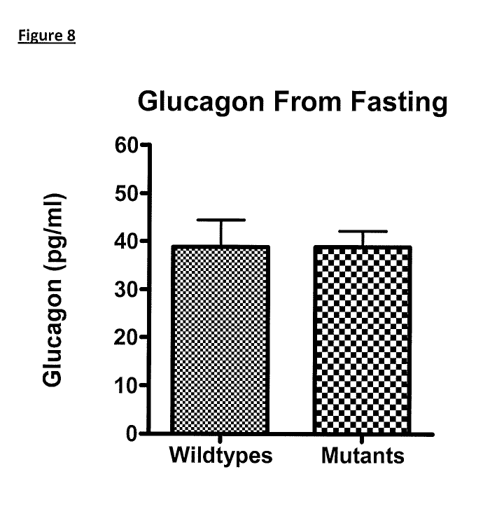

As shown in FIG. 8, there is no difference between mutants and wildtypes. Therefore in the presence of reduced glucose levels the mutants do not show an increase in glucagon, which would normally be expected in a hypoglycemic state. This has led to the hypothesis that the animals are not able to make the switch to fatty acid oxidation once the supply of glycogen is exhausted.

### Example 9

### Biological Data: Glucose/Insulin Tolerance Test

Glucose tolerance and insulin secretion are measured in overnight fasted (16 hour) mice following intraperitoneal injection with 2 mg/g (dose/gram body weight) glucose. Basal blood glucose is measured with a OneTouch Glucometer (LifeScan) and a 50 μl blood sample taken from the tail for insulin measurement. This sample is allowed to clot for 30 minutes at room temperature and then centrifuged as previously. Each animal (n=7) is then challenged with glucose and then glucose measurements preformed at 15, 30, 60 and 120 minutes post injection. At 60 minutes post injection another blood sample is removed from the tail for insulin measurement; this is prepared using the same methods as for the basal sample. At the conclusion of the experiment a terminal blood sample is taken as described for the 12 hour fasting trial above. See Guerre-Millo, M., et al. (2001) “PPAR-α-null mice are protected from high-fat diet-induced insulin resistance.” Diabetes 50: 2809-2814.

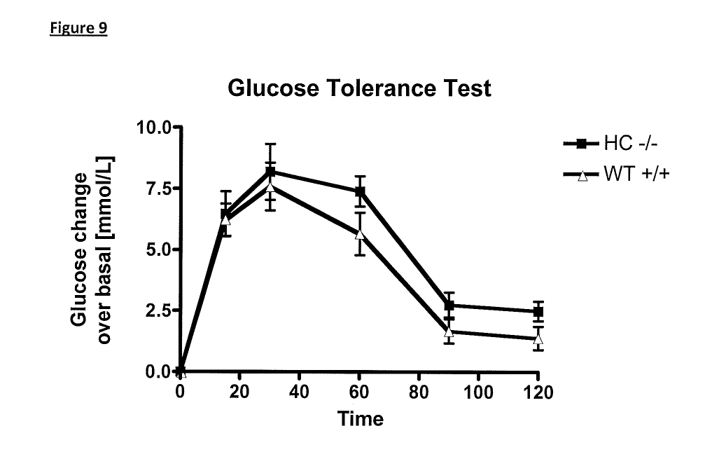

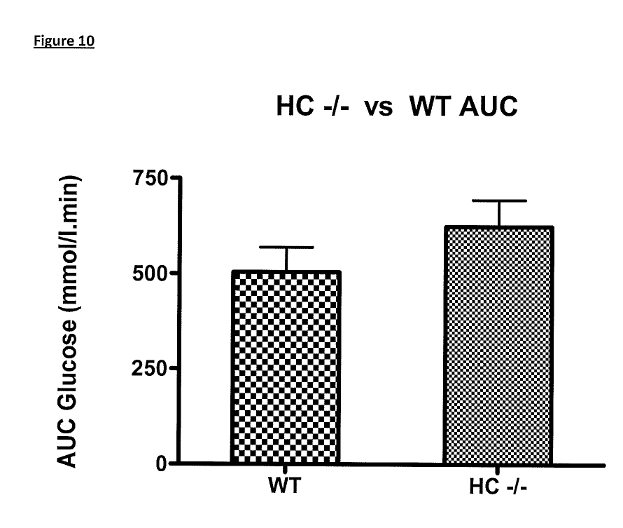

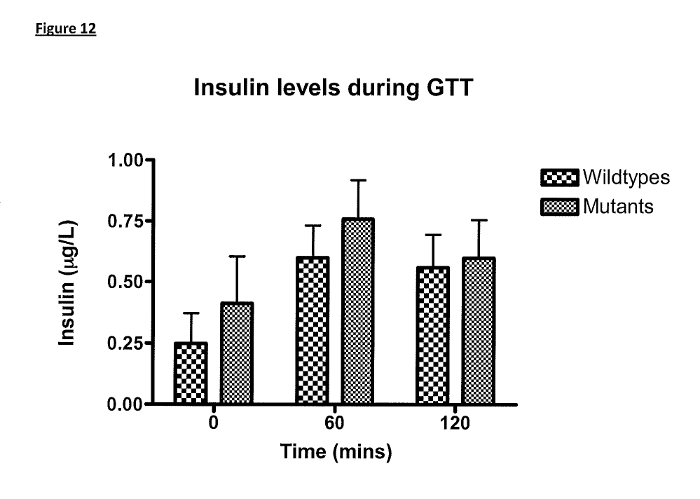

The results are shown in FIG. 9, FIG. 10 and FIG. 12.

The data are normalized for each animal individually by subtracting the basal glucose measurement from all other measurements to adjust for differences in basal glucose levels. The glucose tolerance test reveals no differences between the knockout and wild type animals.

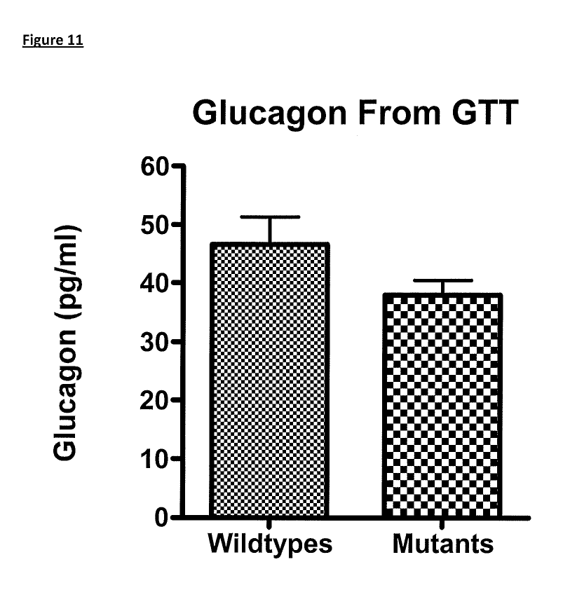

RIA analysis of glucagon levels (measured as above) in the terminal blood sample shows that the mutants do not have a significantly altered level of glucagon. The results are shown in FIG. 11.

ELISA analysis of insulin levels shows that insulin levels rise to a higher levels in mutants than wildtype animals, showing that the mutants are hyperinsulinemic.

In summary, Gpr100 deficient mice show abnormalities in their fat and glucose metabolism after a high caloric diet, either high in fat or in carbohydrates. These findings indicate that Gpr100 is involved in the regulation of energy homoestasis. Similar to the high fat diet experiment high caloric density diets induce obesity and type 2 diabetes in humans. Therefore it is concluded that modulation or interference with the signaling mediated through Gpr100 has a potential for the treatment of diabetes or obesity.

Reference: Steneberg, P., et al. (2005) “The FFA receptor GPR40 links hyperinsulinemia, hepatic steatosis, and impaired glucose homeostasis in mouse.” Cell Metabolism 1: 245-258.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

SEQ ID NO: 1 shows the cDNA sequence of human Gpr100.

GAGAAGCACTTAATTCTACAGCCTCCTTCCTAGAGCCTTCAGTGGCCTC
TGCCAGTCTGGCAGACACTTGCAGACCTCTCTTCTCAGCACCACCAATC
TCTGATGCCCTGCGATGCCCACACTCAATACTTCTGCCTCTCCACCCAC
ATTCTTCTGGGCCAATGCCTCCGGAGGCAGTGTGCTGAGTGCTGATGAT
GCTCCGATGCCTGTCAAATTCCTAGCCCTGAGGCTCATGGTTGCCCTGG
CCTATGGGCTTGTGGGGGCCATTGGCTTGCTGGGAAATTTGGCGGTGCT
GTGGGTACTGAGTAACTGTGCCCGGAGAGCCCCTGGCCCACCTTCAGAC
ACCTTCGTCTTCAACCTGGCTCTGGCGGACCTGGGACTGGCACTCACTC
TCCCCTTTTGGGCAGCCGAGTCGGCACTGGACTTTCACTGGCCCTTCGG
AGGTGCCCTCTGCAAGATGGTTCTGACGGCCACTGTCCTCAACGTCTAT
GCCAGCATCTTCCTCATCACAGCGCTGAGCGTTGCTCGCTACTGGGTGG
TGGCCATGGCTGCGGGGCCAGGCACCCACCTCTCACTCTTCTGGGCCCG
AATAGCCACCCTGGCAGTGTGGGCGGCGGCTGCCCTGGTGACGGTGCCC
ACAGCTGTCTTCGGGGTGGAGGGTGAGGTGTGTGGTGTGCGCCTTTGCC
TGCTGCGTTTCCCCAGCAGGTACTGGCTGGGGGCCTACCAGCTGCAGAG
GGTGGTGCTGGCTTTCATGGTGCCCTTGGGCGTCATCACCACCAGCTAC
CTGCTGCTGCTGGCCTTCCTGCAGCGGCGGCAACGGCGGCGGCAGGACA
GCAGGGTCGTGGCCCGCTCTGTCCGCATCCTGGTGGCTTCCTTCTTCCT
CTGCTGGTTTCCCAACCATGTGGTCACTCTCTGGGGTGTCCTGGTGAAG
TTTGACCTGGTGCCCTGGAACAGTACTTTCTATACTATCCAGACGTATG
TCTTCCCTGTCACTACTTGCTTGGCACACAGCAATAGCTGCCTCAACCC
TGTGCTGTACTGTCTCCTGAGGCGGGAGCCCCGGCAGGCTCTGGCAGGC
ACCTTCAGGGATCTGCGGTTGAGGCTGTGGCCCCAGGGCGGAGGCTGGG
TGCAACAGGTGGCCCTAAAGCAGGTAGGCAGGCGGTGGGTCGCAAGCAA
CCCCCGGGAGAGCCGCCCTTCTACCCTGCTCACCAACCTGGACAGAGGG
ACACCCGGGTGA

SEQ ID NO: 2 shows an open reading frame derived from SEQ ID NO: 1.

ATGCCCACACTCAATACTTCTGCCTCTCCACCCACATTCTTCTGGGCCA
ATGCCTCCGGAGGCAGTGTGCTGAGTGCTGATGATGCTCCGATGCCTGT
CAAATTCCTAGCCCTGAGGCTCATGGTTGCCCTGGCCTATGGGCTTGTG
GGGGCCATTGGCTTGCTGGGAAATTTGGCGGTGCTGTGGGTACTGAGTA
ACTGTGCCCGGAGAGCCCCTGGCCCACCTTCAGACACCTTCGTCTTCAA
CCTGGCTCTGGCGGACCTGGGACTGGCACTCACTCTCCCCTTTTGGGCA
GCCGAGTCGGCACTGGACTTTCACTGGCCCTTCGGAGGTGCCCTCTGCA
AGATGGTTCTGACGGCCACTGTCCTCAACGTCTATGCCAGCATCTTCCT
CATCACAGCGCTGAGCGTTGCTCGCTACTGGGTGGTGGCCATGGCTGCG
GGGCCAGGCACCCACCTCTCACTCTTCTGGGCCCGAATAGCCACCCTGG
CAGTGTGGGCGGCGGCTGCCCTGGTGACGGTGCCCACAGCTGTCTTCGG
GGTGGAGGGTGAGGTGTGTGGTGTGCGCCTTTGCCTGCTGCGTTTCCCC
AGCAGGTACTGGCTGGGGGCCTACCAGCTGCAGAGGGTGGTGCTGGCTT
TCATGGTGCCCTTGGGCGTCATCACCACCAGCTACCTGCTGCTGCTGGC
CTTCCTGCAGCGGCGGCAACGGCGGCGGCAGGACAGCAGGGTCGTGGCC
CGCTCTGTCCGCATCCTGGTGGCTTCCTTCTTCCTCTGCTGGTTTCCCA
ACCATGTGGTCACTCTCTGGGGTGTCCTGGTGAAGTTTGACCTGGTGCC
CTGGAACAGTACTTTCTATACTATCCAGACGTATGTCTTCCCTGTCACT
ACTTGCTTGGCACACAGCAATAGCTGCCTCAACCCTGTGCTGTACTGTC
TCCTGAGGCGGGAGCCCCGGCAGGCTCTGGCAGGCACCTTCAGGGATCT
GCGGTTGAGGCTGTGGCCCCAGGGCGGAGGCTGGGTGCAACAGGTGGCC
CTAAAGCAGGTAGGCAGGCGGTGGGTCGCAAGCAACCCCCGGGAGAGCC
GCCCTTCTACCCTGCTCACCAACCTGGACAGAGGGACACCCGGGTGA

SEQ ED NO: 3 shows the amino acid sequence of human Gpr100.

MPTLNTSASPPTFFWANASGGSVLSADDAPMPVKFLALRLMVALAYGLV
GAIGLLGNLAVLWVLSNCARRAPGPPSDTFVFNLALADLGLALTLPFWA
AESALDFHWPFGGALCKMVLTATVLNVYASLFLITALSVARYWVVAMAA
GPGTHLSLFWARIATLAVWAAAALVTVPTAVFGVEGEVCGVRLCLLRFP
SRYWLGAYQLQRVVLAFMVPLGVITTSYLLLLAFLQRRQRRRQDSRVVA
RSVRILVASFFLCWFPNHVVTLWGVLVKFDLVPWNSTFYTIQTYVFPVT
TCLAHSNSCLNPVLYCLLRREPRQALAGTFRDLRLRLWPQGGGWVQQVA
LKQVGRRWVASNPRESRPSTLLTNLDRGTPGZ

SEQ ID NO: 4 shows the open reading frame of a cDNA for Mouse Gpr100.

TAGACCAACACCCAGATTCCAAGGGCTCTTCTAAGAGCTCTCCTGAGAC
AACAGCGGCGGCGGGTGGTTGCTTGCCAGGCCGGAAGGCGGGCACTCCC
TGGTTCCTCTGCTCTGCTGTGCTCTAGCAACCTCCGCGGTCTTGCGATG
GCCACATCCAATTCTTCTGCCTCTCTGCCCACCCTCTTCTGGGTCAATG
GCTCTGGAGACAGCGTGCTGAGCACTGACGGTGCTGCCATGCCTGTCCA
GTTCCTTGTTCTGAGGATCATGGTTGCACTGGCCTATGGACTTGTAGGT
ATCATTGGCTTGCTGGGAAATTTGGCCGTACTGTGGGTTCTAGGTAACT
GTGGTCAGCGTGTGCCCGGCCTGTCTTCTGATACCTTTGTCTTCAGCCT
GGCTCTAGCAGACTTGGGGCTGGCCCTTACTCTCCCTTTCTGGGCAACC
GAGTCAGCAATGGACTTCCACTGGCCTTTCGGAAGTGCCCTCTGCAAGG
TAGTCCTGACCACCACCGTCCTCAGCATCTATGCCAGCACCTTCCTAAT
CACAGCACTGAGTATCGCGCGATACTGGGTGGTAGCCATGGCTGTGGGA
CCAGGTAGTCACCTCTCAGTCTTTTGGGCCCGTGTGGTCACCCTGGCAG
TGTGGGTGGCAGCTGCCCTGGTGACTGTGCCCACAGCAATCTTTGGGGC
TGAAGTTGAGTTGTGGGGCGTGTGCCTCTGTCTTCTGCGTTTCCCCAGC
AGATACTGGCTGGGAGCTTACCAGCTACAGAGGGTAGTTCTGGCCTTCA
TCGTGCCCTTGGGAGTCATTACCACCAGTTACCTGCTGCTGTTGGCCTT
TCTAGAGCGGCAGCAAAGATGCAGGCCACGACAATGGCAGGACAGCCGA
GTGGTAGCCCGCTCTGTCCGTGTCCTGGTGGCTTCCTTCGCCCTCTGCT
GGGTTCCCAACCATGTAGTCACTCTCTGGGAAATTCTGGTAAGGTTTGA
CCTGGTGCCCTGGGACAGTACTTTCTACACCTTTCATACTTACATCCTT
CCCATCACCACCTGCTTGGCCCACAGCAACAGCTGCCTCAACCCTGTGA
TCTATTGTCTCCTGCGGCGGGAGCCCCAGCAGGTTCTTGTCAGCTCCTT
CAGAGCTCTCTGGTCAAGACTGTGGCCTCAAAGGAAGGCCTGCATGGAA
CAAATGGCCCTCAAGGAGGTAGGCGGGAGAACGGTAGCCAGCACCCAGG
AGAGTGGCTCTTCTAGGACACACACAAACACAATGGAACACCTGGATGA
AGGATGCAGCCTGAACACTCTCCTTTCTGAGACCTATCAGGGGCAGAGC
CCACAGATTCTAGGGAGGAGCAGCTGCTCTCTCAGTCAGGCTGCTGTGT
CCCCAGGAGAAGTCTGA

SEQ ID NO: 5 shows the amino acid sequence of Mouse Gpr100.

MATSNSSASLPTLFWVNGSGDSVLSTDGAAMPVQFLVLRIMVALAYGLV
GIIGLLGNLAVLWVLGNCGQRVPGLSSDTFVFSLALADLGLALTLPFWA
TESA1VIDFHWPFGSALCKVVLTTTVLSIYASTFLITALSLARYWVVAM
AVGPGSHLSVFWARVVTLAVWVAAALVTVPTAIFGAEVELWGVCLCLLR
FPSRYWLGAYQLQRVVLAFIVPLGVITTSYLLLLAFLERQQRCRPRQWQ
DSRVVARSVRVLVASFALCWVPNFIVVTLWEILVRFDLVPWDSTFYTFH
TYILPITTCLAHSNSCLNPVIYCLLRREPQQVLVSSFRALWSRLWPQRK
ACMEQMALKEVGGRTVASTQESGSSRTHTNTIVIEHLDEGCSLNTLLSE
TYQGQSPQILGRSSCSLSQAAVSPGEVZ
SEQ ID NO: 6
TTGTGCAGAGTTCAATGGAGAATGTTG-
SEQ ID NO: 7
CCAGAAACACTCTACGCCTGTCACCTG-
SEQ ID NO: 8
TttgcggccgcAAAGTGACTCATGCTGCTCCCATCTTC-
SEQ ID NO: 9
AaaactagTCCCAGCAAGCCAATGATACCTACAAG-
SEQ ID NO: 10
TttggcgcgCCTGGGACAGTACTTTCTACACCTTTC-
SEQ ID NO: 11
TttggccggccTCCATTTAAGAAGAGATCTTGAGCCAG-
SEQ ID NO: 12
TGGATCCTTTTATTTTGGAGACTGAAC-
SEQ ID NO: 13
CCTGGCTCAAGATCTCTTCTTAAATGG-
SEQ ID NO: 14
GGTGAGCAATCAGATCATGAGACTTAC-
SEQ ID NO: 15
GCTTACCAGCTACAGAGGGTAGTTCTG-
SEQ ID NO: 16
TGATGGGAAGGATGTAAGTATGAAAGGTG-
SEQ ID NO: 17
GTCGTGACCCATGGCGATGCCTGCTTG-
SEQ ID NO: 18
CGGATCCACTAGATAACTTCGTATAGC-

SEQ ID NO: 19: Genomic Locus from 5′prF to 3′prR

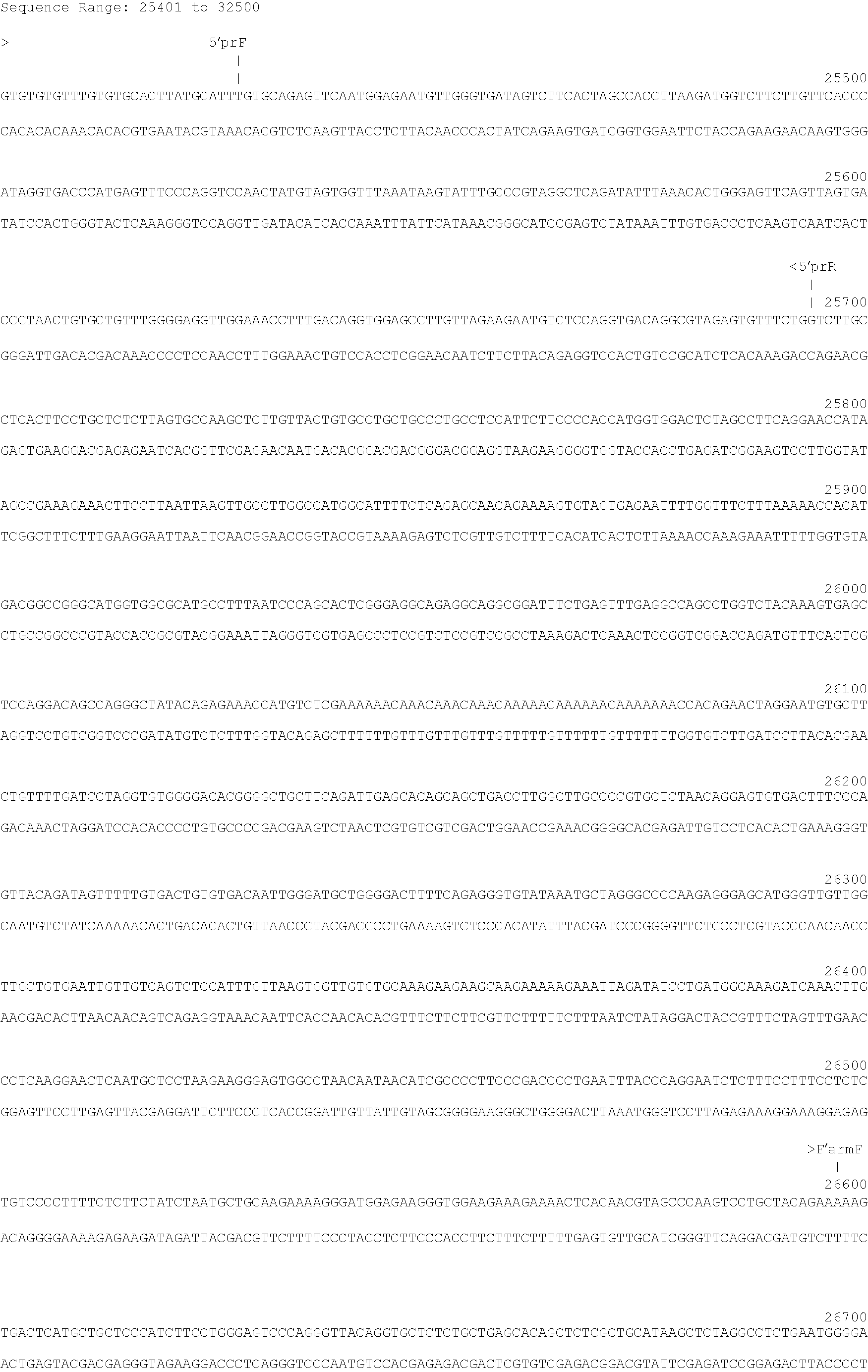

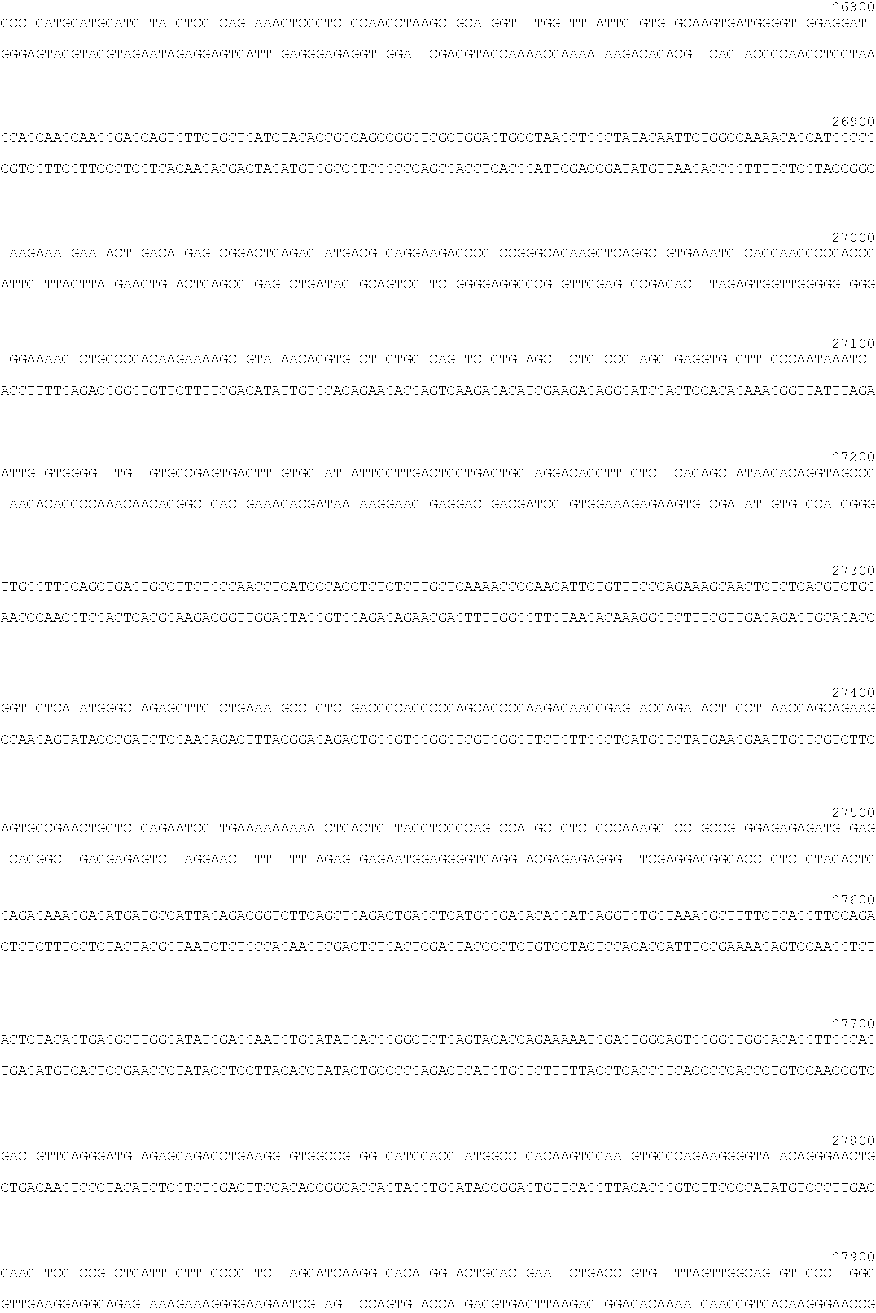

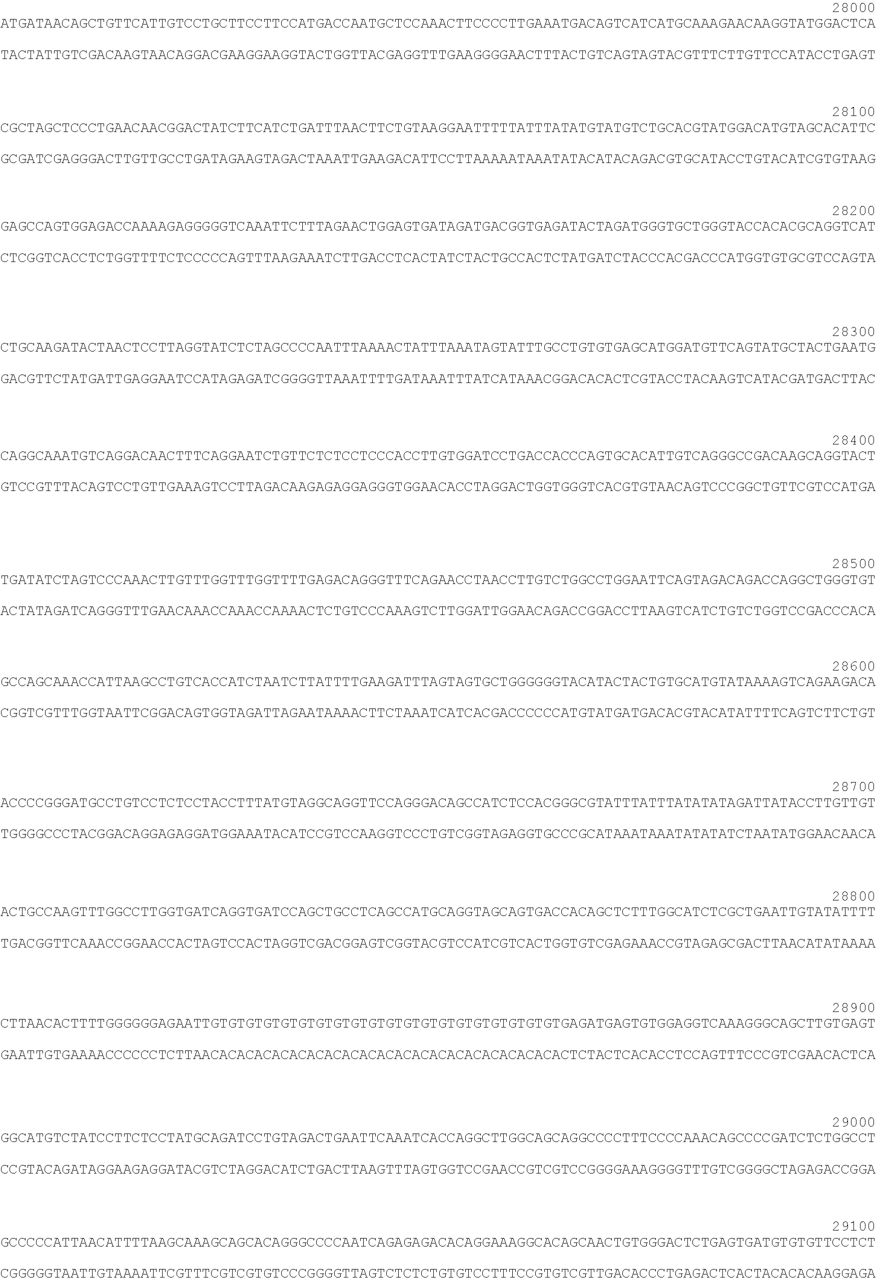

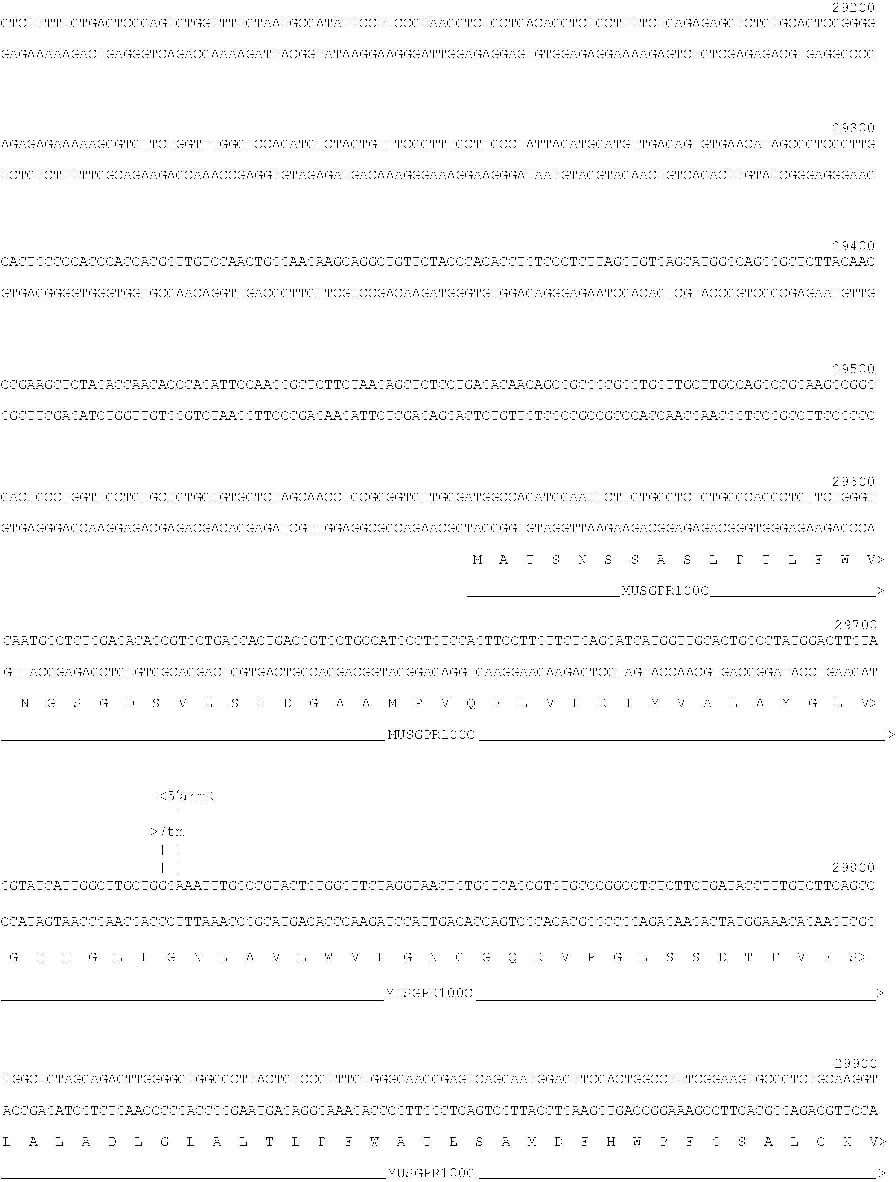

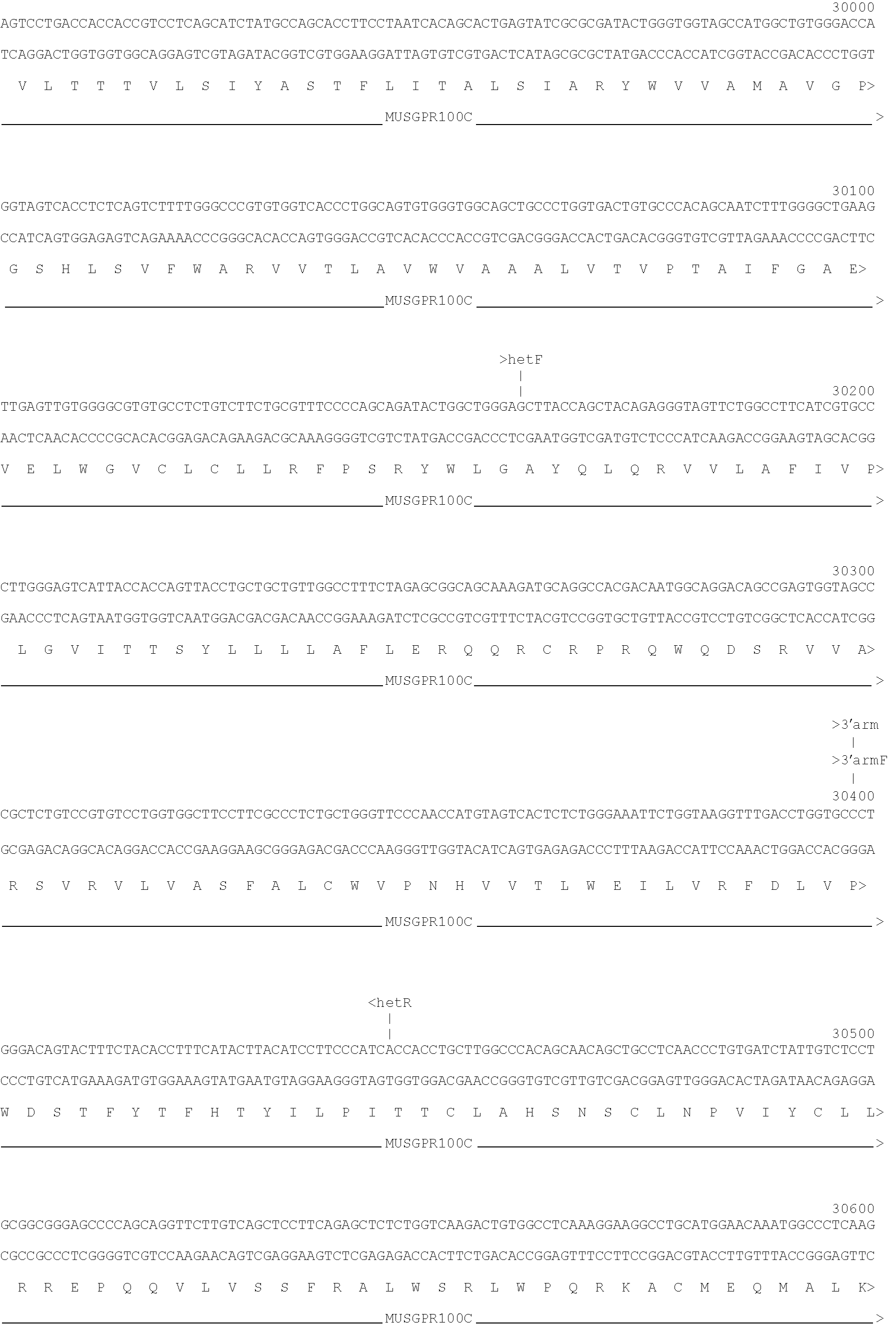

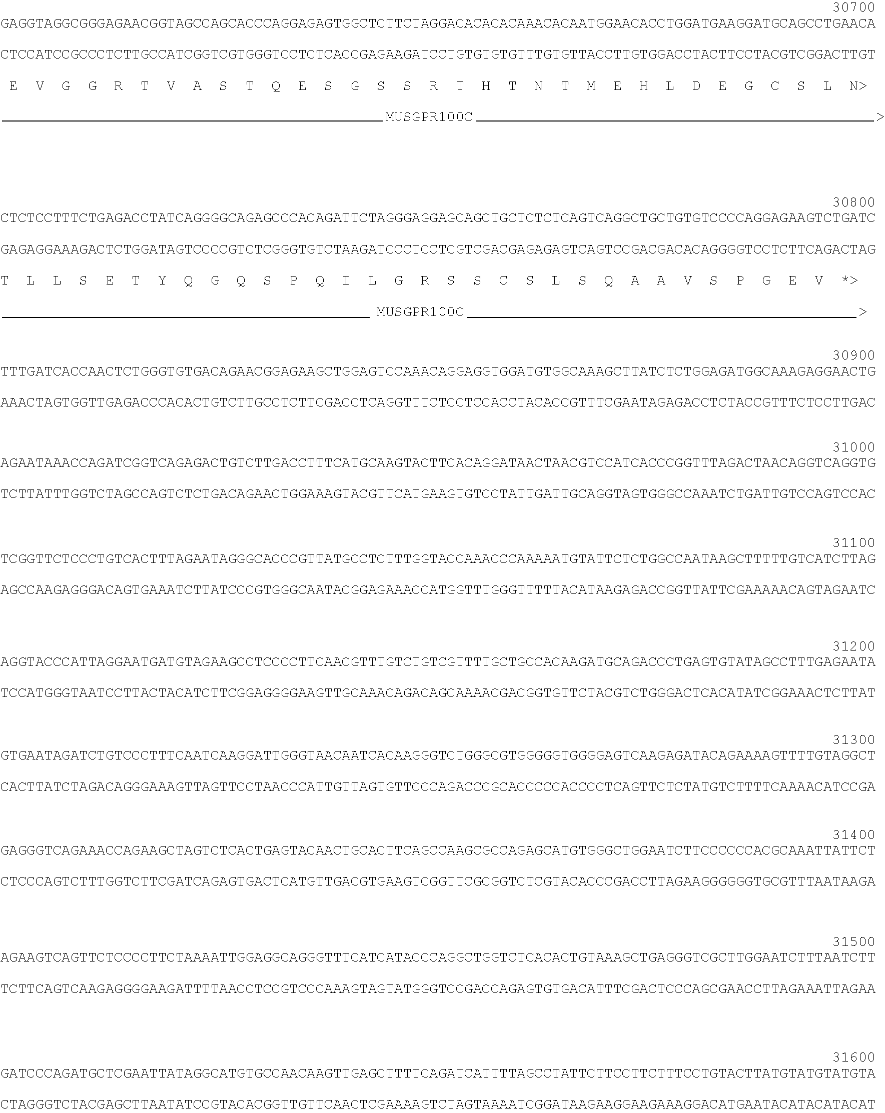

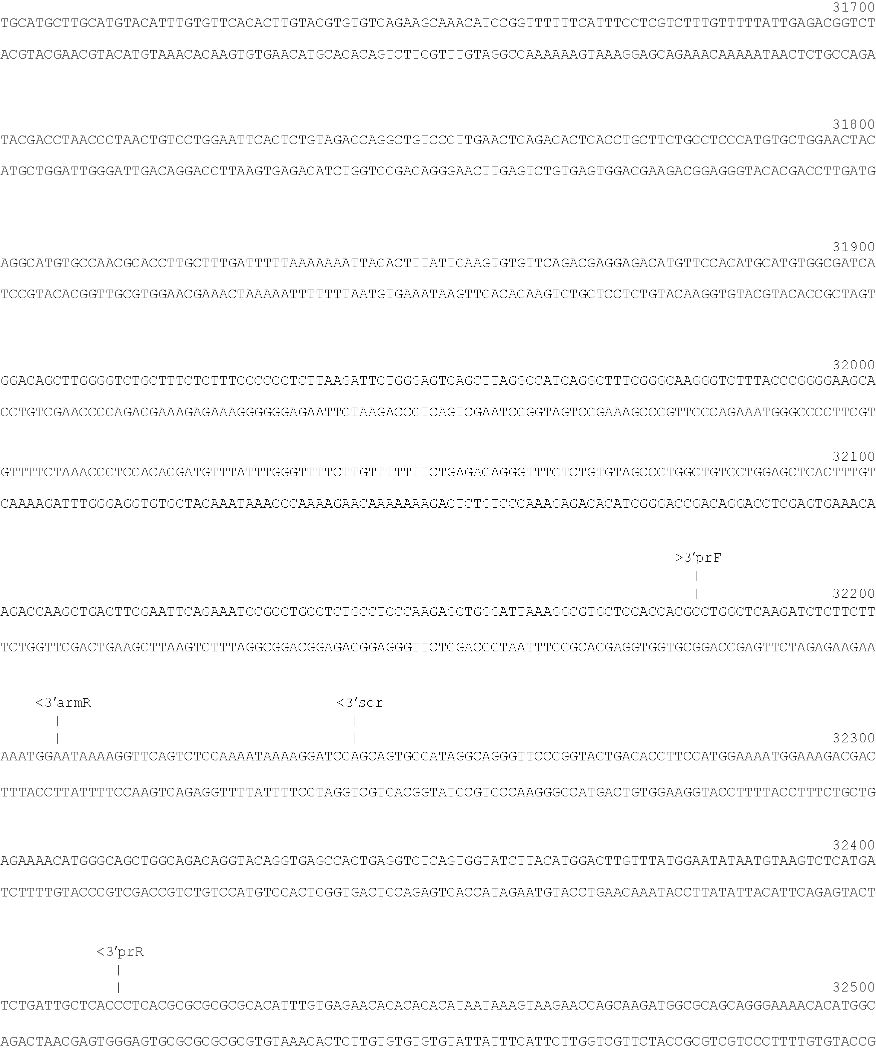

1. A method of identifying a compound for treating, preventing or alleviating obesity or diabetes in an individual, wherein the obesity or diabetes is associated with activity of Gpr100, the method comprising:
(a) contacting a functional Gpr100 polypeptide with a candidate compound; and
(b) determining whether the candidate compound binds to the Gpr100 polypeptide and acts as an antagonist of the Gpr100 polypeptide.

2. The method of claim 1, wherein the Gpr1100 polypeptide comprises an the amino acid sequence shown in SEQ ID NO: 3 or SEQ ID NO: 5 or a sequence having at least 90% sequence identity thereto.

3. The method of claim 1, wherein the compound is an antibody.

4. The method of claim 1, wherein the candidate compound is exposed to a cell expressing a Gpr100 polypeptide.

5. The method of claim 4, wherein a change in intracellular cyclic AMP (cAMP) or calcium levels is detected.

6. The method of claim 5, wherein a decrease in intracellular cyclic AMP (cAMP) or calcium levels is detected, thereby identifying an antagonist of Gpr100.
